(19) United States
(12) Patent Application Publication
(10) Pub. No.: US 2020/0375493 A1
(43) Pub. Date: Dec. 3, 2020

(54) Universal ECG Monitor

(71) Applicant: VLADIMIR KRANZ, Prague 4 (CZ)

(72) Inventor: VLADIMIR KRANZ, Prague 4 (CZ)

(21) Appl. No.: 16/759,802

(22) PCT Filed: Aug. 31, 2018
(86) PCT No.: PCT/CZ2018/000041

§ 371 Date: Jul. 17, 2020

(30) Foreign Application Priority Data
Aug. 31, 2017 (CZ) PV 2017-510
Sep. 27, 2017 (CZ) PV 2017-592
Dec. 29, 2017 (CZ) PV 2017-861

(51) Int. Cl. A61B 5/0408; A61B 5/00; A61B 5/044; A61B 5/0428

(52) U.S. Cl. CPC A61B 5/04085; A61B 5/6831; A61B 5/044; A61B 5/6823; A61B 5/0006; A61B 5/681; A61B 5/04286; A61B 5/6824

(57) ABSTRACT

This invention relates to a universal ECG monitor of cardiac signals with enhances possibilities of scanning. The prior art monitors of cardiac signals make possible either a short-time touching by fingers or by other part of the human body of the electrodes, or they are permanently for a long time by means of a chest belt or by other means attached to the human body, wherein, such means are impractical and non-emergency. The equipment according to the present invention is a universal ECG monitor of a compact and easily portable design, which monitor substitutes the prior art costly and with difficulty movable hospital equipment. Moreover, it is a very economical solution, which makes possible to obtain a complete set ECG curves, i.e. the 12-lead ECG, by means of one of the monitor subsequently moved into various positions, and so to scan signals from different parts of the human body.

### FIELD OF THE ART

This invention relates to a universal monitor of cardiac signals.

### PRIOR ART

The prior art monitors of cardiac signals allowed either a short time touching of electrodes by fingers or by some other part of the human body, or the electrodes were fastened by means of a chest belt or by some other means to the human body for a long time. But they did not allow to use the monitor for the short-term, medium-term and also long-term tests.

These drawbacks were removed and an improvement represented a universal monitor with external power supply for monitoring of cardiac signals, which has been disclosed in the PCT/CZ Application No. 17/000012, WO 2017/021767. But still it was not possible to modify the number of leads with which such device operated.

Further, such devices from the state of the art monitor and evaluate cardiac signals by a monitor designed as a card, which card is used in the short-term tests by attaching it to the chest or the card is touched by fingers, but such devices do not allow to carry out the long-term or the permanent monitoring.

The prior art desktop monitors do not allow their fastening to the bottom part of the mobile phones or to smart watches in such way that they would be fastened permanently.

The monitors used in the relatively small cards do not allow both the temporary extension of the spacing of the contact areas to achieve improved sensing from the chest and the connecting to an additional accumulator to increase capacity of the small card battery. Also, it does not allow their removable inserting into the phones.

A monitor of cardiac signals can be wirelessly linked with a mobile phone, which phone is carried for example in a pocket, or it is possible to put the monitor on a mobile phone. In case of the most frequent position of the mobile phone, in a pocket, the display is not visible. It would be preferable to have some operative instant view, in particular during displaying of the health data, for example by placing it on a wrist, what would be more convenient than to extract it out of a pocket.

Putting of a monitor on a mobile phone carried on a wrist or on some smart watch, as it is in the prior art, is not possible, and also, it is not possible to take the mobile phone out of a pocket or take the smart watch out of a wristband on a wrist to attach it to ear for making a call, in particular when they are connected with the monitor.

### SUBJECT OF THE INVENTION

The universal monitor disclosed in the PCT/CZ Application No. 17/000012 is developed further by this invention disclosed in this application. It is about a universal monitor of a compact, easily displaceable design, which universal monitor substitutes the prior art costly and with difficulty replaceable devices, such as for example the hospital devices. Moreover, this universal monitor is a very economical solution, because with it is possible to obtain up to twelve-lead ECG curves using only one chest belt, preferably comprising a monitor, which monitor is preferably gradually moved to different positions or is switchable, and thus it allows sensing of signals from different body parts. To expand the number of leads, probes are used that are placed on straps, clips and/or they are glued, which supplement the probes fitted directly on the chest belt in an optimal configuration within a curve. The signals from the electrodes are led to the chest belt, and they are brought to individual positions for providing with a monitor, which monitor then scans the signal, processes it and transmits it further or stores it in a memory. According to the number of monitors provided and the positions an ECG signal is obtained within the required number of leads at once or subsequently, and this from the required positions for scanning of the cardiac signals. The operative system for measuring of the ECG consists of a combination of pieces of equipment comprising a system of electrodes, a chest belt, a monitor of cardiac signals, a notebook, and a printer, fastened to it by means of mounting elements of the printer, and further of a case fastened to the printer or to the notebook by means of a fastening mechanism of the case. This case serves to store parts necessary for ECG scanning. The whole assembly is provided with a withdrawable loop, which loop is used for the possible easy carrying. Preferably, the mounting mechanism is comprised of double angle irons, wherein, the smaller internal one is moveable by sliding with regard to the external one, whereby, it is achieved that notebooks of different, but only the same or smaller sizes as is that of the printer can be connected to the printer.

The panel monitor, which can be attached to the chest, comprises two electrodes. These electrodes, together with the RA. LA, Rl, and LL electrodes make a multi-lead ECG measuring possible, which measuring is carried out sequentially so that the panel monitor is sequentially attached to three places, wherein during the first attachment the leads V1. V2 are monitored, during the second attachment obliquely the leads V3 and V4 are monitored, and during the next attachment the leads V5 and V6 are monitored.

The chest belt, which is fixed on the chest muscles, does not allow measuring of the common leads. However, the groups of electrodes deployed on the belt symmetrically to the chest bone make possible to select probes of different sizes and placements by their interconnecting and combinations. By interconnecting of electrodes on both sides, two large-area collecting electrodes—the Kranz's clip—are obtained that make possible to monitor a specific ECG lead, which is providing a significant amount of information.

Scanning of all leads by means of the chest belt is made possible by a segment of electrodes, which segment is fitted permanently or withdrawably to the chest belt, and which segment provides the position of electrodes for the leads V1 to V6 in correct positions. The probes for the leads RA, LA, RL, LL are monitored by glued electrodes or by electrodes on clips or straps. Alternatively, the segment of electrodes is larger and it comprises also electrodes for these leads. Preferably, the segment of electrodes is of metal and is pre-tensioned as a spring, so that after it is pressed to the chest by the belt pressing of all electrodes to the chest is ensured, and thus the corresponding conductive connections to the human body surface. Alternatively, for the LA, RA leads suspenders are used that press the electrodes to the human body surface in the shoulder areas that is in the areas of the trapezoidal muscles, and for the LL. RL leads a side belt is used, which side belt presses the electrodes to the human body in the areas in the region of the pelvis top on both sides of the human body.

In the standby mode, the monitor preferably monitors only the heart rate and the characteristics derived from it, including the arrhythmia, and for monitoring and sending of the ECG it is switched on only in the case the limit values are exceeded, and/or on the basis of an instruction issued by the monitored person or by the surveillance center or by an authorized remote participant.

Other configurations of the monitoring system allow to the monitored person to have maximum comfortable feeling even when it is driving in a means of transport or is operating some other machine, further only generally the means. The system comprises a universal monitor disclosed in the PCT/CZ Application No. 17/000012, WO 2017/021767. This monitor is placed on the shortened chest belt, or alternatively, in the backrest or in another part of the means. The cardiac signals are monitored by means of the collecting electrodes that are deployed on the shortened chest belt, and connected with the monitor. In contradiction to the solution disclosed in the PCT/CZ Application No. 17/000012, the system for monitoring is enhanced by adding the capability to scan when the monitored person is dressed not only into a shirt or blouse, but also into a T-shirt.

In addition, the novel solution allows also partial movement of the monitored person, such as its turning to a side etc. Most often, the monitor is placed on the shortened chest belt, which belt the monitored person puts behind the shirt. A bridge is attached to the chest belt inseparably or separably, wherein, the spikes of the bridge pass through an opening provided in the shirt towards the chest. A template is put on the spikes and the template is pressed by the belt. The template passes on the pressure exerted by the pressure exerting belt to the spikes, but the template is movable to sides with regard to the spikes so that when the monitored person turns to a side, the position of the shortened chest belt is changed with regard to the template under permanent force action of the pressure exerting belt towards the spikes, and thus to the shortened chest belt. Alternatively, pressure exerting belt is oriented horizontally and it is anchored in the backrest with winches on both sides and/or it is an oblique safety seat belt of the means. If the monitored person is wearing a T-shirt, it does not click a bridge to the shortened belt, it clicks to it a fixation element, usually a small trough. The person attaches the belt under the T-shirt towards the chest and above the T-shirt it inserted a fixation counter-piece into the fixation element, in case of the small through it is a fixation block. The fixation counter-part is fastened to the pressure exerting belt by the template, but it is movable with regard to the pressure exerting belt in the horizontal directions. If the monitor is placed on the shortened belt, the electrodes are interconnected with the monitor by conductors that are located directly in the belt. If the monitor is placed anywhere else, preferably in the backrest and/or in another part of the means, the conductors connect are routed through the cross-connecting bridge to the pressure exerting belt and to the monitor. Preferably, the monitor, which is placed on the chest belt, is powered by means of the conductors in the pressure exerting belt and in the bridge. This power supply solution allows smaller dimensions of the monitor. Alternatively, the monitor is designed as one powered from an external accumulator connected to it through a connector and/or it is clicked on similarly as the monitor placed on the shortened chest belt, and the power supply is carried out by means of the conductors routed from the accumulator to the monitor that are placed in the belt. Alternatively, the accumulator, either integrated in the monitor, clicked on and/or externally deployed, charges a contactless one, inductively so that an induction charger is fastened in sufficient vicinity of the accumulator in its operative position.

The monitor for monitoring of persons operating any means, for example a transportation means, is placed in the dashboard, or in the seat body or on the shortened chest belt. It can be placed withdrawably in any place by means of a mounting mechanism, preferably by snap fasteners, and it communicates either wirelessly or by wires. In case of communication by wires the conductors are routed via connectors. If the monitor is placed on the shortened chest belt, either permanently or withdrawably, preferably, it is placed off its center and a bridge is placed in the shortened chest belt, wherein, the bridge connects the chest belt with the pressure exerting belt mechanically disconnectably or permanently, preferably also with the safety belt. At the same time, the bridge provides with its connector connection of the monitor preferably with a power source from the electric system of the means and connection for signals with the controlling and displaying module. Preferably, the conductors pass through the pressure exerting belt or they pass in parallel with it to the winch, so that always only the necessary length is taken out. In case the power supply of the monitor is provided from the external source, preferably an electric system of the means and/or of the wired communication, the monitor can be of substantially smaller dimensions. Preferably, such reduced monitor can be provided also with a supplement of the monitor comprising an accumulator and a module for wireless communication. The supplement is connected to the monitor directly by means of a connector or it is fastened to the chest belt, through which the links are routed to the monitor.

The monitor and/or the supplement of the monitor are powered preferably inductively so that an induction charger is fastened to the pressure exerting belt so that the pressure exerting belt will be placed as tightly as possible to the monitor and/or the supplement of the monitor.

In case of the wired connection of the monitor, when the bridge with connector interconnecting is not used, the conductors are brought by a connector directly from the monitor or from the bridge, and by a cable they are brought to the connector placed on the pressure exerting belt, and further they are brought to the power supply and to the controlling and displaying module.

Another improvement is the wider scope of scanning consisting in adding of the scanning of signals of the left arm, the right arm, the left leg, and the right leg by means of probes, preferably formed by clips and/or wristbands that press the sensing electrodes that are their parts to the wrists and ankles, or to other parts of limbs. Preferably, said clips are integrated in the driver's seat, from which seat they move out for fastening them into the working positions. After releasing from the working position on a limb the supply cable is wound to a clamp, it is wound, preferably it is wound by a self-winding mechanism, into the seat body and the clip is put into the clip holder. Preferably, the cables are routed via the auxiliary straps, wherein the straps reduce the pulling force of the winches and hold the cables along the limbs. The signals of four electrodes for scanning of the limb signals transmitted to the monitor in accordance with its deployment, either on the belt or in the driver's seat and/or in another place, preferably in the dashboard of the means. If the monitor is placed on the shortened chest belt, the signals are transmitted to the monitor via the conductors that are integrated in the pressure exerting belt, wherein the conductors are routed into the bridge, and further into the monitor. Alternatively, the signals are brought by conductors into the connectors placed on the shortened chest belt and/or they are brought directly into the monitor. To obtain a multi-lead, up to twelve-lead ECG, it is possible to use the shortened chest belt disclosed in the PCT/CZ Application No. 17/000012, which belt comprises electrodes for scanning of the leads V1 to V6. The shortened chest belt can be connected to the pressure exerting belt by means of a bridge disconnectably or preferably permanently, what allows, in case the belt is not used, when the driver disengages the pressure exerting belt, rolling up of the pressure exerting belt, together with the bridge and the chest belt, by means of the winches, wherein they are rolled into the rest position on the seat side.

The above described configuration allows scanning of the one-lead ECG, either from the chest belt by using the collecting electrodes and/or from the signals of the upper limbs. If a multi-lead ECG is required, the signals of the lower limbs LL, RL and those of the chest belt, allowing scanning of the leads V4, V5, V6, are added to the signals/leads of the LA, RA. The twelve-lead ECG is thus obtained, when the chest belt with electrodes in a curve is used.

The thin design of the monitor having the size of a credit card provided with a small accumulator, the contact surfaces for ECG scanning from fingers and/or after attaching this card to the chest, is preferable for scanning of the ECG from the chest in case of the short-time scanning. This monitor comprises a storage medium used to store the measuring. The monitor comprises a control unit, a communication module, control push-buttons, electrodes, and preferably contacts used for charging. For an optimal scanning of the signal from the chest, such electrodes are too near to each other. To optimize this scanning, preferably, the monitor is provided with a tipping or a pull-up contact feature or with plates provided with contact areas being the electrodes, and their distance is greater after they are positioned into a position for measuring from the chest. Alternatively, the plates are withdrawable and they are mechanically attachable by putting then on, or snapping them on, or some other connecting, to produce a complete assembly ready for scanning.

The monitor of a thin design can be inserted into a case hanging on a fastening tape, with which tape it can be fastened to the chest of the monitored person to carry out the ECG scanning. The case allows access to the control push-buttons and/or to the contents by the control push-button, and it is interconnected with the monitor electrically. Preferably, the case is adapted for inserting of an additional accumulator to allow a longer monitoring time period. Then, it is possible to provide middle-time recordings. Alternatively, some modular additional accumulators can be connected to the case or to the monitor that can be fastened to the fastening belt and they can be interconnected with each other and with the monitor to allow power supply for a long time. Thereby, a long-time ECG recording is possible. Alternatively, the modular additional accumulators are connected together by stretchy segments and so they form the whole fastening tape, or a part of it. The monitor of thin design, placed in a case, can be fastened to a wristband. In this design, the case is provided with a covering of one contact area, and with routing it to the upper part of the case. The uncovered contact area is in contact with the wrist of a hand, and the other contact area, which is routed to the upper side of the case, is to be used for contacting it with the other hand by touching it with a finger.

The monitor in the thin design, the reduced one, i.e. the one without any power supply and wireless communication part, is attachable both mechanically and electrically by means of a connector to a controlling and displaying module, preferably to a mobile phone. Alternatively, the electrical connection is realized by a cable.

When the supplement of the reduced monitor is connected to it, features the monitor are enhanced by a longer operation time and the wireless communication.

Alternatively, the reduced monitor is inserted into the monitor base, which monitor base further enhances its features. An external accumulator of higher capacity can be connected to the monitor base by means of some fastening elements in any suitable place. The power supply electronics of the base for recharging allows also inductive charging.

The reduced monitor of thin design can be enhanced by a supplement and by another board with electronics for the possibility to connect an additional accumulator.

Further, the drawbacks of the prior art equipment are solved by the monitor according to the present invention application, wherein the monitor is designed as a card of smaller dimensions that monitors cardiac signals, wherein the monitor is fastened to the controlling and displaying module, preferably formed by a mobile phone, or it is fastened to a controlling and displaying operative module with the wristband, preferably formed by a mobile phone on a wristband or by a smart watch, wherein it is fastened by means of a fastening means.

The communication is wireless. During communication by a wired connection of the monitor with the connector of the module, an accumulator, a communication module, controlling elements, and an extractable memory medium of the module are used, and preferably, the parts are not placed in the monitor, whereby, it is a sensor of the monitor, which is provided only with the front end, which is monitoring the cardiac signals received from the contact surfaces on the sensor or the module and it is provided also with a control unit.

In case of the monitor mounted, preferably underneath the operation module on a wristband or a wrist watch, preferably, in case of the monitor of the desktop design, it overlaps said modules or watch, and thereby, the contact surface for touching it by a finger of the other arm is accessible, wherein, this finger preferably touches the contact area on the bottom side of the monitor board on the wrist of the first arm, what is used for scanning of cardiac signals. With a cross-connecting element towards the contact areas of the monitor, the monitor is adapted to scan cardiac signals from the other arm by means of an electrode, preferably by means of an electrode glued to the chest or placed on a wristband placed on the other arm. The cross-connecting element is connected by wires to these electrodes by means of a contact area from the wrist of the first arm. Thereby, the monitor is adapted for the long-term monitoring of cardiac signals. For the long-term scanning, the monitor is adapted also by the fastening it to the chest belt, preferably through contact surfaces, by means of a fastening means, preferably formed by a holder.

Preferably, the monitor or the sensor of the monitor are adapted for withdrawable placing in the controlling and displaying module or in the operative controlling and displaying module on the wristband, preferably by insertion or preferably by means of a door or snapping it to it. If necessary, the monitor is also adapted for extracting for independent use for monitoring, for example by connecting it to the chest belt or to another equipment mentioned in the “Examples of Embodiments of the Invention”. Cardiac signals, scanned and processed by the monitor, are transferred to the module or to the operation module, and preferably, said modules communicate together and they pass on information about the processed cardiac signals or phone traffic.

In the modules the processed cardiac signals are displayed and/or stored, preferably in a replaceable memory and/or they are transmitted into a distant server by means of mobile network or internet, with access rights for remote users.

In case of an independently operating monitor the data are stored preferably in a removable storage medium, from which it is preferably wirelessly transmitted by means of a communication module or they are transmitted by repositioning of the storage media into a local or remote PC or into a server for further processing.

The mentioned drawbacks of the prior art are removed by a monitor according to this invention application by connecting of the monitor to a displaying and controlling operative module preferably formed by a mobile phone or by a smart watch, preferably placed withdrawably, preferably by means of the base on a strap on the wrist. Preferably, on this base also the monitor is mounted, preferably withdrawably, wherein the monitor is adapted for wireless or electrical connection, preferably by means of a connector. In case, it is placed together with the module on the base or the monitor is adapted for wireless communication or wired communication by means of a cable with connectors with a module for communication in case it is placed remotely with regard to the operative module. The displaying and controlling module is adapted for placing it on the base, wherein, it is fixed by a tape on the wrist preferably withdrawably. Preferably, the monitor is adapted for inserting or connecting it by means of a cable, of an additional accumulator, preferably placed on an arm or on a wrist. The monitor is adapted for connecting it to the contact of the first arm, which contact is preferably formed by an electrode on the bottom part of the base or alternatively on the chest belt or it is a glued probe on the chest, which replaces the contact of the first arm on the bottom of the base, and it is connected by a cable to the connector in the monitor.

Preferably, the contact of the other arm is formed by an electrode and it is placed on the top part of the monitor case or preferably, it is formed by a cable with a connector into the monitor of electrodes for the wristband of the other arm, or by a glued electrode on the chest or on the chest belt.

In case of connection of an additional accumulator, the alternative contacts of the first and/or of the other arm are preferably connected through the connector in the additional accumulator, which is connected with a connector between the additional accumulator and the monitor. Preferably, the monitor is placed on the base withdrawably and it can be relocated, preferably together with an additional accumulator to the base of the chest belt, through which base it is electrically and mechanically connected with the electrodes of the chest belt. This monitor processes and sends further the cardiac signals scanned by the electrodes of the first and the other arms. They are processed to at least one result from the following ones: ECG, curve and/or value of the heartbeat, preferably with the limits of the regular heartbeat, the curve and/or the values of arrhythmia, the values and/or the curves of variability.

The operative controlling and displaying module, preferably of smaller dimensions, serves for operative monitoring from the wrist. Preferably it is connected wirelessly with a displaying module, preferably one of larger dimensions, for detailed displaying for data transfer for mutual communication and mutual displaying of the displays, the so called mirroring, or for displaying according to some selection, for example of information about incoming calls, SMS. Preferably, the modules are adapted for mutual control, receiving calls, SMS messages between themselves. For receiving calls the operative module is withdrawable off the base and can be attached to ear for managing of voice calls. It is provided with a headphone and a microphone.

For managing of phone calls when the operative module is placed on the wrist, it is provided for the “hands free” traffic.

Preferably, the monitor of cardiac signals is put on withdrawably, e. g. on the wrist, or the monitor is connected by a cable and it is fastened on the wrist remotely from the operation module.

Preferably, the operation module is fastened on the wrist withdrawably to allow using of the for calling by attaching the phone to an ear, or it is adapted for loud phone calls with a “hands free” fastening on the wrist.

Preferably, the monitor of cardiac signals is connected by a connector, through which it communicates and obtains power without installation of some own accumulator or a communication module.

When the module for independent operation is disconnected, the monitor of cardiac signals connects to the base provided with an accumulator and a communication unit.

Preferably, the monitor of cardiac signals is provided with a replaceable accumulator.

For a longer period of time without charging the monitor is connected to an additional accumulator, which is put on the monitor and which is connected by a connector or which is placed remotely on the wrist and connected by a cable.

Preferably, the monitor is connected withdrawably to the basic plate, to which basic plate it is connected, preferably withdrawably, also the module, wherein the basic plate is fastened to a wrist by the wristband or the basic plate is fastened to the wrist and pressed to it by the module, which is provided with a wristband for fastening it on the wrist.

For a one-lead ECG of the first type of lead, the monitor scans cardiac signals on the first and the other arm by means of the electrode of the first arm, which is placed on the wrist of the first arm from the bottom of the monitor or the basic plate and for touching by a finger or the wrist of the other arm on the top or on a side of the monitor.

For alternative scanning of signals of the other arm, the monitor is adapted for connecting of through the connector by a cable to the electrodes placed on the wristband on the other arm or placed on shoulder or on chest near the shoulder of the other arm.

For alternative scanning of signals of the first arm, the monitor is adapted for connecting through the connector by a cable to the electrodes on shoulder of the first arm or on chest near the shoulder of the first arm.

For scanning of cardiac signals associated from the center of the chest, the monitor is adapted for connecting by means of a connector by a cable of electrodes placed symmetrically on the left and the right half of the chest, preferably under the level or at the level of the nipples by gluing or placed on the chest belt encircling the chest by means of strap preferably an electric one or on the shortened chest belt without any strap or on a plate held on chest.

The monitor provided with a source and a communication module in the body of the monitor or in the base is preferably adapted for wrist removal and for independent operation by touching with fingers or with wrist of the first and the other arm to the electrodes placed on it, or it is adapted for connecting through a cable of electrodes of the first and the other arm or the associated signals, or for repositioning to the chest belt or the plate or to the shortened chest belt with electrodes for scanning of associated signals from the chest.

The monitor is adapted for repositioning, preferably to an independent wristband for scanning of signals on the first and the other arm z from the electrodes to the monitor and/or plate of the module or for scanning from external electrodes connected by a connector and by a cable. Preferably, the module is a mobile phone or a smart watch or an operation unit provided optionally with parts for mobile phone and/or for smart watch.

The monitor can be fastened on the module operatively on the wrist or on the controlling and displaying module off the wrist, preferably of larger dimensions, interconnected by means of a connector or a cable or placed in it so that it can be extracted, preferably by means of a connector and/or a door or a foldable cover or placed removably or placed permanently.

For preliminary scanning of cardiac signals for general determination of a deviation from the standard cardiac activity, in particular in case of scanning during motion or from limbs, curve of ECG with essential SW, to filter off interference so that the curve of ECG is as much as possible aligned to a straight line on the X axis with the QRS complex and the T wave, with the result that they are not entirely distinct, the P wave and another, e. g. Small fibrillation waves. For detailed scanning at rest the curve of ECG is damped off and it is transmitted with all or with majority of details, in particular for scanning from the electrodes on the chest for the one-lead scanning with adding of signals from limbs for multi-lead scanning.

Preferably, the monitor on the wide chest belt is fastened withdrawably by means of mounting elements connected to the electrodes, where a part of electrodes is in this case preferably placed on the chest belt and forms a system of electrodes on the belt and a part of electrodes is preferably fastened on the body of the monitored person and forms a system of external electrodes either by adhesive target, preferably withdrawably with conductive gel or by suckers or by fastening straps or on arms and on the thighs or by wristbands or by clips on the wrists and ankles and it is connected by wires, and by their communication a system of electrodes comes to existence, which is easier to install on the body of the monitored person then to install individual electrodes. The above mentioned on the thighs placed electrodes is an example of a 12-lead ECG, and for different use and number of leads the number of electrodes and their arrangement can be adapted.

The narrow chest belt may be in the design with three electrodes in the system of electrodes on the chest belt and four electrodes in the external system of electrodes. The monitor may be for a three-lead application expandable up to a nine-lead one.

For a three-lead application, the electrodes RA, LA, LL, RL, V4 are connected to the monitor and the output are the measured ECG curves for leads I, II, V4. By connecting of electrodes, preferably by a selector switch of electrodes, to the electrode V5 and then V6 the ECG record is enriched with curves from the leads V5, V6 that are measured subsequently and added to the record for application of 5 leads measured. By calculation of the leads III, aVR, aVL, aVF the 9-leads application is obtained. The output signal is transmitted by wired link through the connector of the chest belt or by a wireless link from the monitor to the evaluation unit, which unit is formed preferably by the controlling and displaying module or by the operative controlling and displaying module or by a mobile phone or a tablet or a notebook or a PC, from which it is transmitted preferably to the server by means of a WiFi network 131 or a mobile operator's network via internet, and from there to the remote participants. The wirelessly transmitting from the monitor is realized by means of a wireless module, which module is adapted alternatively for transmission via the WiFi network 131 or the mobile operator's network directly to the server. The presented example of the system of electrodes is preferably a seven electrodes comprising one, wherein, three electrodes are placed on the chest belt.

Preferably, this system is usable for a Holter with recording to an extractable memory medium that is portable to the evaluation unit for the downloaded record. Instead of the selector switch the monitor for measuring also of the leads V5, V6 is usable alternatively. Preferably, the selector switch is formed by a three-position mechanical selector switch or by a portable jack or selector switch DIP or by an electronic selector switch.

The monitor may be repositioned to the chest belt or placed on it for the 12-lead ECG.

Six electrodes is placed in the system of electrodes, preferably on the wide chest belt loosely attached to the chest or fastened by the encircling pressing elastic band, which band allows layout of electrodes on the axis that is optimal for scanning of the 12-lead ECG. Other four electrodes in the system of external electrodes are connected by wires.

Preferably the monitor is placed withdrawably on the belt in the array of positions and is adapted for scanning of up to 12-lead ECG at once, or a monitor is used that is adapted for scanning of only a part of the leads. In case of adaptation for six leads the monitor is repositioned after scanning of the six leads in the position 1 to the position 2 for scanning of another six leads, i.e. of twelve leads together. In case of adaptation to four leads, there are three repositions to the positions 1, 2, 3 for scanning of a 12-lead ECG. Similarly, when monitors are used for scanning of 3 leads, they are repositioned 4-times to the positions 1 to 4, in case of two leads, they are repositioned 6-times to positions 1-6, in case of 1 lead they are repositioned 12-times. In case of any repositioning, the monitor is fastened by mounting elements, preferably with connectors for connecting to the respective electrodes.

An advantage of the repositioning of the monitor to the connectors of the chest belt is saving for a cheaper monitor, for example the one used for only four or six leads. In case, the belt is used for scanning of the same number of leads as the monitor, preferably from the array is used only one connector and the rest is not necessary to install or the monitor is connected to the belt permanently.

The system of electrodes with six electrodes in the system of electrodes on the chest belt up to four electrodes in the system of external electrodes outside the chest belt, connected by wires to the belt, exhibits the advantage that it is easy to install the system for scanning of ECG and then to de-install it as a whole, in comparison to individual electrodes connected by wires to the monitor or to an evaluation unit. Moreover, transmitting from the monitor to the unit directly from the electrodes to the belt or electrodes connected to it through the connector of electrodes, see FIG. 1, allows easier installing than as it is in case of connecting the evaluation unit directly to individual electrodes by wires. The monitor placed on the belt is connected by wired m link or wireless link to the evaluation unit, preferably formed by a notebook or a PC or a mobile phone, wherein, the 12-lead system of electrodes is connected directly by wires, without any monitor to evaluation unit and preferably, it is used for providing of an ECG on a unit, preferably formed by a desktop ECG instrument, comprising a front end or a notebook or a PC that are supplemented by the front end circuits, preferably placed in a dongle. In this way, the evaluation unit is adapted for data processing of cardiac signals for displaying or storing in memory or printing in usual form of the ECG preferably with diagnosis and the ECG parameters. Preferably, by that, this 12-lead system will replace the common ECG, either by interconnecting of the 12-lead system to a desktop ECG apparatus or to a notebook or PC.

The 12-lead system is preferable for monitoring of events that can be uncovered only with difficulty on an ECG with less leads, for example the heart attack the sites of ventricular extra-systoles or of bigeminias. During a long-term scanning it is preferable to display on the display of the evaluation unit, preferably the module, information about cardiac activity, preferably arrhythmia or occurrence of a ventricular extra-systole and number per time unit for evaluation of cardiac activity, preferably for athletes and monitored persons with heart disease, so that these persons can modify their regime on a continuous basis.

The monitor may be used on the narrow chest belt for the one-lead ECG, where the electrodes are formed preferably as the collecting electrodes, which are longer and placed on another place than the usual ECG electrodes and they scan the aggregate signal for more leads ECG, for example for leads I, II, and V1-V6, and other, whereby allow a summary overview about all leads in a simple way from the one-lead narrow chest belt, and thus the assessment in a simple way degree of imbalance of the ST sections that are important in the evaluation of the heart attack or angina pectoris, in particular under load, for what would be necessary a more complicated ECG, for example a 12-lead one. If necessary, the monitor is repositioned to the belt adapted for scanning of the multi-lead ECG by repositioning the monitors in the array of positions into various positions, and/or into only one position for the required number of leads. Repositioning of the monitor from a belt for a lower number of leads to a belt with a higher number of leads and to other positions saves in comparison to a more expensive monitor, which would allow scanning with more leads, for example with 12 leads, without repositioning and which would be more complicated than the one-lead monitor and which would be moreover used only sometimes, so that the costs spent for it would not be fully used. And moreover, the multi-lead belt is bigger, and thus it is more comfortable to wear the smaller belt with lower number of leads, when more leads are not necessary. The more leads having monitor is used, the more simple is the handling, with a lower number of repositioning required, but at the expense of higher costs. Scanning without repositioning allows the monitor adapted for scanning of 12 leads. By snapping of the monitor into the array of positions connecting is realized that allows evaluation of 12 leads.

Therefore, one position is enough, the other ones are not necessary to be realized.

The multi-lead monitor may be repositioned within an array of positions into positions, for example 1 to 3 positions, or the respective electrodes can be gradually switched by the selector switch from the system of electrodes to the monitor, for providing, for example, of the 12-lead ECG.

For example on the unit, in this example formed by a notebook, the application“providing of a 12-lead ECG” is set. The display displays the instruction “place the module to the position 1, press START” After recording of the curves for the first position the instruction“Place the module to the position 2 and press START” is displayed. And so it is proceeded further, till all 12 leads are displayed and sent. Preferably, at first all leads are recorded in the unit. Thereafter, communication with the server or the PC is established for their sending. Alternatively, the record is used to record gradually automatically during repositioning of the monitor to the positions 1-3, and when the record is complete, it is sent to the unit or the server. In case of the manual control the push-button for sending for making a record is pushed after each repositioning of the monitor for record and push-button of sending after recording of each record to the memory medium. When the selector switch is used data is sent about the respective lead at each switching to another position. Alternatively, the record is recorded to exchangeable memory medium from which data is transmitted by repositioning to the selected unit or server.

Preferably, on the display of the evaluation unit it is possible to follow the recording quality, which recording is repeated if necessary.

On the unit or on the server after transmission of all leads the leads are modified into the proper format, either the usual 6 leads one under the other and 6 other leads next to them, or into another selected format.

Preferably, parameters of the recording are also displayed, for example heart rate, QRS complex, etc., and preferably also the diagnostics, for example the heart attack or the AV block of grade 2.

The printer may be removably placed underneath the notebook for the purposes of the ECG for printing of ECG reports directly from the notebook. After tilting away of the monitor of the notebook the keyboard is accessible, preferably with a mouse. This layout is suitable in particular for hospitals, where according to the prior art a non-portable ECG instrument is transported to the patients.

The notebook with the printer are easy to carry and allow operative carrying to the patients.

The printer may be attachable to the notebook by means of a mounting mechanism of the printer and the connector of the printer.

A closed notebook with an attached printer and a detachable loop-strap may be carried easily.

Preferably, on the printer is by means of a case mounting mechanism attached a case provided with storage space accessible by a door to be used for storage of the monitor or of the chest belt, of electrodes, and of other accessories for the purposes of the ECG.

It is possible to form a set of a notebook with a printer by means of some mounting mechanics, preferably formed by a universal mounting mechanism. Preferably, the universal mounting mechanism is formed by a double angle iron attached to a side of the printer providing the keyboard part of the notebook, over which the monitor is folded, in the required position to the printer. The double angle irons are adjustable in certain extent and allow so mounting of the notebooks of different sizes, not exceeding the dimensions of the printer. With this printer, they are electrically connected by a cable with connectors, preferably some USB for data and/or power transfer.

The belt with a monitor may be used in the physician's waiting room or in an ambulance in the hospital. Patients are monitored at the time of waiting. Data are stored in a preferably, extractable memory medium for later transfer of data, in particular of the ECG data, on a physician's PC.

The physician will so use the patient's waiting for providing of a Holter type recording, which is processed from the data stored. Preferably observed phenomena are recalculated from the actual recording time to 24 hours, which period of time is the usual for Holter recording. Or the data is transmitted wirelessly already during the waiting time period directly or via a mobile data network, preferably a WiFi one, alternatively, via an evaluation unit for example via WiFi, on a physician's PC, preferably formed by a notebook or Internet via a server.

The physician can monitor the patient's health during the waiting period of time and, in case of a deterioration of the condition, the physician should examine the patient at risk with preference. Preferably, the states being off limits are reported by an alarm and preferably the data and the ECG of the monitored person with data off limits are automatically displayed on the physician's PC.

In the PC it is possible to set displaying of more patients, that can be switched as needed.

Also in this case, preferably, from the transmitted data a Holter record is processed in the monitor or in the evaluation unit or in the PC or in the server. The physician can give instruction to record the monitored data also from the medical office on a PC. Recording by touching the belt or the monitor contacts with fingers or palms is also possible instead of placing of the monitor fastened to the belt on the patient's chest.

The evaluation unit can communicate, and preferably it comprises a notebook and a monitor, which is preferably put into the connector for connecting of the monitor to the notebook, preferably formed as a USB, preferably placed on the front side of the notebook for easy attaching of fingers to the electrodes, so that by connecting of the monitor a preferable configuration for scanning of the ECG of the monitored person by means of electrodes is obtained and at the same time for observing of the record on a display of a notebook via a monitor connector, which communicates and is powered so that preferably in the monitor there is not any accumulator as well as any communication wireless module. This makes the monitor smaller and cheaper. Alternatively, the monitor is connected by means of an extension cable into the connector placed from the side. The cable can be used also for connecting to a mobile phone. Another alternative is a wireless link, preferably a Bluetooth, for communication from the monitor. In comparison to a communication through the connector or cable it requires to add into the monitor an accumulator and a communication module. The wireless interconnection allows also connecting to an evaluation unit, preferably formed by a PC. The notebook and/or the PC is provided with some software for data processing of cardiac signals received from the monitor for 1-lead to 12-lead ECG, preferably in the ECG format and/or the Holter format and it will fully replace the common ECG instruments or the Holters. Preferably, the reports are printed on a printer or they are stored in memory. The transmissions of data of the cardiac signals may be provided also from the server or by repositioning of the portable memory medium, preferably a SD card, storing data stored there preferably in the monitor.

The system of electrodes may be on the chest belt, or on external devices wired to the monitor with an s evaluation unit.

In this embodiment the monitor is a part of the belt. The monitor comprises a front end and a control unit of the monitor. Together with the evaluation unit, preferably formed by a notebook, the monitor is connected by wires through the connector chest belt by a cable and/or wirelessly by a wireless link.

The chest belt implementation may be also without any monitor with the same systems of electrodes, in which the signals of all electrodes are brought together into a connector of electrodes in the belt. Through this connector, the signals of electrodes are brought by means of a multicore cable to the circuits of the front end in the monitor. The control unit of the monitor processes the signals transmits them to the evaluation unit, preferably formed by a notebook. The monitor is plugged into the connector or into the evaluation unity, or it is integrated in it.

Alternatively, by a multicore cable, the signals are brought to the front end of the desktop ECG, and the signals are processed by the control unit of the ECG.

The chest belt can comprise a connector for connecting of the monitor, preferably formed by snap fasteners. Signals of the left collecting electrode and the right collecting electrode are transmitted to the monitor. The left collecting electrode can be connected by a withdrawable bridge with the electrode for scanning of the cardiac signal from the recommended place for placing of electrodes for scanning of the ECG signal V4.

The multi-lead chest belt may be adapted for mounting of up to 9-lead monitor. The connector of the monitor is adapted for bringing of signals from the RA probe, the LA probe, the RL probe, the LL probe and the V4 probe, the V5 probe, the V6 probe. According to the model of the monitor, this monitor scans leads I, II, V4, V5, V6 at once and/or the leads I, II and the leads V4, V5, V6 are subsequently switched by means of a selector switch of the V-leads. The collecting electrodes are not used.

The belt may be enlarged by a withdrawable additional board, which allows scanning also of the lead V1, the lead V2, and the lead (the Kranz's clip) V3, what is implemented so that the to them corresponding electrodes are placed on the desk in positions above the chest belt so that they closely fit to the respective places of the chest of the monitored person after fastening of the belt. The required pressure of electrodes to the chest skin is achieved so that the plate is enlarged also to the opposite side of the chest belt and is pre-tensioned. This embodiment allows, according to the model of the fitted monitor, scanning of the simultaneous 12-lead ECG or subsequent ECG, where the leads V1 to V6 are switched subsequently by means of a selector switch of the V-leads.

The chest belt is in the embodiment, where the additional board is snapped to the belt, which additional board, however, allows to connect also the external electrodes directly to it, i.e. the RA probe, the LA probe, the RL probe, the LL probe. The board is fastened to the belt by means of a connector of the monitor, and it is pressed to the chest of the monitored person by a belt. Further, the board comprises a monitor comprising circuits for processing of up to 12-lead ECG. This configuration allows a very rapid transfer from the informative measuring belt without any additional board of a 1-lead to 4-lead measuring to the complete 12-lead ECG by mere snapping of the additional board and fixing of the RA, LA, RL, and LL probes.

Instead of the enlarging plate for scanning of the leads V1 to V3, the chest belt can comprise three independent springs fitted in the upper ends by electrodes for scanning of the leads V1, V2, V3. Said leads supplement electrodes for scanning of the leads V4, V5, V6 that are placed directly on the chest belt. Said 6 electrodes form the system of electrodes of the belt. In cooperation with the system of external electrodes for scanning of the lead I between the electrodes RA and LA and the lead II between the electrodes LA and LL, together, they form the whole system of electrodes. Therefore, the monitor provided on this belt can monitor eight leads and the four remaining up to the full 12-lead ECG are only calculated.

Preferably, the belt comprises a position for an additional accumulator placed on the belt and used to prolong the operation time of the monitor. By this, functionality of the monitor accumulator is prolonged and/or backed up.

Preferably, the chest belt is provided with collecting electrodes, particularly with the right collecting electrode and the left collecting electrode, and with a monitor of the cardiac signals, wherein this monitor is placed on the chest of the monitored person and used to scan the 1-lead ECG. Alternatively, a universal chest belt may be used, which is provided with electrodes and a monitor used for scanning the multi-lead ECG, wherein, either the collecting electrodes or the electrodes for the multi-lead ECG can be used optionally. Optionally, the belt is attachable to the chest and it is held there, or it is provided with stretchable straps for fastening to the chest by means of some mountings. The collecting electrodes are placed in places, where the common ECG electrodes are not placed usually, and what they scan does not correspond to any lead of the common ECG, but they are to some extent like the lead I covering partially the lead V3 to the lead V5. An advantage is that they display as a summary the S-T section, which is partially produced from more leads.

The chest belt may be used for fastening of the attachable monitor for example for scanning of the 8-lead to 12-lead ECG. For fastening, preferably, the monitor fastened by snap fasteners is removed from the belt and the chest belt is moved from the position under the nipples serving for scanning of the one-lead ECG to the position for scanning of the multi-lead ECG so that its chest electrodes are in the correct position, preferably between the 4th and the 5th rib, centrally against the chest bone. serves The position of the monitor, which monitor is preferably formed by a sensor of the multi-lead ECG, preferably the 8-12 lead one, is secured by a guiding rail of the monitor, which rail is attached to the chest belt by placing of the sensor. After the correct placing of the monitor on the chest so that the electrodes are in correct place, as stated above, the position of the chest belt with regard to the chest nipples, preferably by means of a foldable scale, which is placed on the chest belt, and which is folded off into the measuring position. Preferably, this position is marked on the foldable scale for a next easier placing of the attachable chest belt to the correct position. By activating of the respective push-button on the monitor the scanning of the ECG is activated, which scanning records up to eight-lead ECG by means of a system 3 of electrodes wired to the monitor, used for the left and the right arms and the right leg, preferably supplemented also with the reference electrode for the right leg. Preferably, the ECG record, stored in memory, is transferred to the evaluating unit after activating of the push-button for transfer. In case of making record also for other ECG leads for the chest leads V3-V6, the monitor is transferred to the position for measuring of the leads V3, V4.

A record in the evaluation unit is carried out after activation of the record push-button, what is used for a record of up to eight-lead ECG, incl. The chest leads V1 and V2. In case of repositioning of the chest leads V3, V4, by a repeated pushing of the push-button for transfer also the record for leads V3, V4 is stored in the memory. Similarly, for other repositioning of the monitor for recording of the V5, V6 leads, after their recording the push-button for transfer is pushed again, which is stored in the memory. Thereafter, it is possible to use the record stored in the memory of the evaluation unit for displaying it in this unit. The records of all leads will already be available there. They will be arranged one under the other so as it is usual in a common ECG record. For transmitting to a server, the respective control equipment is activated on the evaluation unit. Preferably, the evaluation unit is formed by a mobile phone or by a tablet or a notebook or a desktop computer or on a displaying unit. In case a tablet is used by the physician, it is not necessary to provide a record, because a record displayed on this tablet is can be carried together with it and it can be presented to the medical personnel so as it is required. From the tablet, as well as from other units forming the evaluation unit, it is possible to provide a record by transfer to a printer or directly from the unit, if such unit comprises a printer, such as for example in case of a desktop ECG apparatus, to which apparatus it is possible to transfer the cardiac signals. When used in the hospital environment, the one-lead ECG is preferably scanned by means of a chest belt, where it is possible by transmitting the signals to the control room, preferably via a local WiFi and a server, to display records of more patients on a screen of a PC, wherein the display is split to an array partial images. In case of the automatic reporting of an alarm with regard to the scanned ECG values from the multifunctional chest belt of some patient y or u exceed the preset limits, the screen automatically displays data of the patient where the limits were exceeded. In this case, preferably, the possibility to monitor the multi-lead ECG is used, wherein, the monitor, preferably formed by a attachable sensor is used, and so the 8-lead to 12-lead ECG is available immediately by mere attaching of this sensor, and preferably by fastening it with the chest belt. It is a much quicker and simple method than transporting of a desktop ECG apparatus on a trolley to the patient, as it is the common practice. An advantage is that the chest belt is already fastened, and thus other handling is not necessary and holding of the sensor by this chest belt is more simple than the holding of the sensor by the medical personnel. Moreover, it allows quicker and more accurate placing of the sensor to the correct place. Also, an advantage is that ECG record is produced in the evaluation unit formed by a notebook, because, in particular in the hospital environment, it is easier transportable than the desktop ECG on a trolley. Moreover, it is not necessary to make a printed copy because the notebook may be carried by the medical staff to the physician to see the record and the printed copy can be made on a common printer if required. The notebook or tablet allow to the physician to see the record directly without printing it and to store it in the memory of the hospital server for the respective patient to see him/her in future.

The monitor may be attached to the chest of the monitored person and keeping of the correct position angle of the monitor with regard to the belt is secured by bridges that are placed on the monitor, through which the belt passes, and which are formed preferably by a U-profile used for securing of the position of the belt. Instead of using the bridge the correct position of the monitor can be drawn, for achieve the correct angle with regard to the belt. For this placing, the belt is preferably placed under the position, in which the chest leads V1 and V2 were measured. For later reference for rapid placing of the belt and of the monitor into the correct position, the foldable scale is used for finding of the shift of the chest belt from the originally measured position, respectively from the nipples, which shift can be marked on the scale for further use.

The monitor may be attached for measuring of the chest leads 5-6, in this case again in the horizontal position, in parallel to the chest belt. The belt is mounted to the monitor by means of the guide rail. After attaching to this guide rail, the chest belt is shifted to the respective position, which position is measured, preferably by means of the foldable scale with regard to the nipples, wherein the measure will be used for future reference. The snap fasteners in the center of the belt serve preferably for correct centering of the belt with regard to the chest axis and the correct placing of the monitor to the belt, and thus to the chest, is set by means of the markings on the chest belt.

Preferably, the monitor is formed by a sensor attachable for scanning of the ECG signal by means of electrodes that are placed on it on its bottom side, which electrodes are provided with contacts to the skin for attaching of the sensor to the chest. The electrodes are made of a conductive material, preferably a metallic one, and they are slightly bulgy to provide for a secure contact, as it may be seen in the view in the direction “D1”. Preferably, the two electrodes may be formed each by two electrodes that are connected for better signal intensity. In this case, the center between these electrodes is taken as the resulting electrode for definition of the place of this electrode on the body. The view D2 shows the monitor in view from the side with the guiding rail, which rail is interrupted preferably and with the bridges for oblique guiding of the belt. On the front panel also the control push-buttons and an indication LED are placed.

The chest belt may be designed as provided with foldable scales around the hinge, wherein the basic position of the chest belt is provided e. g. by a Velcro hook-and-loop fastener. Preferably, marking of the monitor position on the belt is carried out to make more easy repeated measuring, to avoid using of the scale every time again.

Alternatively, the monitor is formed by a sensor attachable to the chest by holding it with a hand without any use of the chest belt. An advantage of using the belt for fastening of the monitor on the chest is the possibility to measure its precise position by means of the foldable scale or by markings 313 on the belt, further of the bridge on the monitor and centering of the belt by means of snap fasteners. In case of the long-term measuring by means of a belt with collecting electrodes with a monitor for one-lead ECG when this belt is already attached to the chest which it arounds, and therefore, its use for holding of the monitor on the chest is not necessary, another advantage is that it is not necessary to place this belt on the chest, instead of it may be used directly for pressing of the monitor during its appropriate shift on the chest. In this way, the handling is simplified in comparison to the one when the chest belt has to be installed.

The chest belt with the collecting electrodes that comprise electrodes that can be optionally connected together by bridges. So it is possible to select one collecting electrode from all electrodes which electrode is of variable size in different positions on each side of the chest belt, or the collecting electrode comprises only a certain number of electrodes.

In case that the collecting electrode on the right part of the belt comprises all electrodes that do not need to be switched, preferably, only one longer electrode is formed without switching. Preferably, on the left side of the belt the electrodes are switched to make possible to associate the electrodes into one larger electrode or to leave them separated for measuring from the electrodes V4, V5, V6 independently, and not as a collecting electrode. The electrodes are switched by a bridge, which bridge connects only the selected electrodes, or by a selector switch of the V-leads. In this belt, the electrodes connected to it can be selected individually as required. An advantage of this configuration is that all electrodes are placed on one narrow belt, in case of measuring against the collecting electrode on the right side of the belt, which may be put together selectively from any number of electrodes placed there, wherein, by this the Kranz's clip is obtained, which is uses in this case instead of the classic Wilson's clip for measuring from the electrodes on the other side of the belt, i.e. the V4, V5, V6 subsequently selected by the selector switch, or at once, according to the number of inputs adapted in the monitor. An advantage consists in being placed on one narrow belt in comparison to the classic Wilson's clip, where it was necessary to place other 4 electrodes off this belt. So measuring of the modified chest signals V4, V5, V6 is obtained.

The chest belt may be in the design, from which now preferably only the electrodes for the chest leads V4, V5, V6 are used because the bridge is separated and the electrodes are connected through the selector switch to the monitor. These electrodes are measured against the classical Wilson's clip comprising of electrodes for the left arm LA and the right arm RA, that are placed on the chest belt under the underarm connected by wires together with electrodes V4, V5, V6 on the selector switch and further on the monitor placed on the side belt towards the waist line, on which are situate the remaining two electrodes of the Wilson's clips for the left leg LL and the right leg RL, also connected to the selector switch by wires. If it is necessary to measure also the V1, V2, V3, the segment of electrodes shown in the detail 1 is connected by a connecting element to the chest belt, placed under the nipples, where the connecting element fastens and simultaneously connects electrically the electrodes V1, V2, V3 placed on it to the system of electrodes. The segment is made of a stretchy material, preferably of a steel sheet, it is slightly convex, as it is shown in the view D, so that when it is pressed by the chest belt it straightens and so presses the electrodes V1, V2, V3 to the skin for good contact. The selector switch is used in the case that a monitor is used, which is adapted for measuring of three leads, i.e. the 1, 2, and V1, wherein, the three leads AVL, AVR AVF are calculated, that is from totally seven leads, by connecting of four electrodes, the Wilson's clip and the V1 or V4. When measuring with the remaining five chest leads, the monitor is switched subsequently by the selector switch for subsequent measuring and recording in the evaluating unit. In case of using the monitor, which is adapted for simultaneous measuring via 12 leads by connecting of 10 of electrodes the selector switch is not necessary. In case when the RL electrode for the right leg is placed off the side belt, the strap of electrodes is used, which is of a stretchy material and slightly bulged again. The system of electrodes placed on three belts is preferable in that all belts are narrow and easy to put on and it is not necessary to use any glued electrodes. All electrodes are already interconnected by wires in this system, so that no subsequent connecting is necessary. To allow separating of the belts for their storing, preferably the wired connections are placed on connectors, and so separable. Preferably, the segment of electrodes and the strap of the electrode are connected to the belts by a connecting element, preferably formed by snap fasteners that not only connect the segment or the strap mechanically but also connects it electrically to the system of electrodes.

The chest belt may be in the design, from which preferably the electrodes, probes V4, V5, and V6 are used for measuring of leads V4, V5, V6 that are subsequently switched by the selector switch of the V-leads to the monitor. To this monitor this selector switch switches also the electrodes allowing to apply potential from the classic Wilson's clip LA, RA, LL, RL, which are placed on the connecting segment of electrodes, which is connected to the chest belt by a connecting element, preferably formed by snap fasteners. This connecting element, where it is apparent that the electrode for V3 is also placed on it. Preferably, this element is made of a stretchy material and it is slightly bulged so that it is flattened when it is pressed by the chest belt, whereby a force is produced acting on the electrodes so that they sit well on the skin. In case the monitor is used, which is adapted for measuring by ten electrodes, that is for measuring of all twelve leads at once, it is not necessary to switch between the electrodes, and the measuring will be carried out at one time. From the monitor the measured data are transmitted into the evaluating unit by wires, or wirelessly or the data are transmitted directly into the server, where they would be otherwise transmitted by the evaluating unit if required. This arrangement is advantageous in that it is possible to use the narrow chest belt for measuring of one lead or alternatively also of the leads V4, V5, V6 by the Kranz's clip, and in case of the classical measuring by the Wilson's clip the same chest belt is used preferably by connecting of the segment of electrodes and by disconnecting of the bridge for selecting of electrodes. So it is achieved that the complete system of electrodes is already mutually interconnected and it is not necessary any other connecting of the individual electrodes, what is an advantage for easy installing. Also, it is an advantage that it is not necessary to use the glued electrodes that are uncomfortable.

The system of electrodes may be the same with a smaller segment of electrodes for easier storing, installation and carrying, for the price that resulting measurement may not be so accurate, as it is in case of placing the electrodes in their classical positions

The system of electrodes, may be with the chest belt placed under the armpit replaced by braces for electrodes for the right arm RA and the left arm LA, by which they are interconnected to the remaining electrodes of the system of electrodes. Said braces are fastened to the remaining two belts, to make a preferable connection of electrodes RA, LAnd this the rest of the system of electrodes possible. Then, the braces are pulled over the shoulders and come back to the chest belt. An advantage of this system is that the belts and the braces are interconnected into one set and completely mutually also electrically connected so that there is no need to interconnect individual electrodes and at the same time the braces hold the belts in the selected position wherefore they are adjustable in length. If it is desirable to measure leads V1, V2, V3 the segment of electrodes is used for placing of the RL off the side belt, the strap of electrodes is used.

Places on the chest, where the electrodes for scanning of ECG signals are placed, are marked as V1 to V6.

The chest belt may be in the design with a monitor under the clothing preferably represented by a shirt or a T-shirt pressed to the chest by the pressure exerting belt preferably comprised of two parts in the middle connected by a buckle, which parts are wound on winches with springs, which, after being pulled out, re-wind the parts of the pressure exerting belt when they are released again back into the winches. Therefore, preferably the pressure exerting belt is comprised of two parts, wherein each of them is wound on one winch placed on both sides of the backrest of the vehicle seat, which belt is connected by a buckle preferably placed in the middle between the winches. This arrangement is adapted to allow free movement of the monitored person controlling the means, what may be a transportation means, a machine and/or a mechanism. When moving forward or sideways, the pressure exerting belt is correspondingly unwound from the winch or it is wound back into it. The springs ensure tensioning of the pressure exerting belt for pressing of the chest belt for scanning of cardiac signals or the belt is pulled out of one winch and it is wound in on the other side of the backrest by a latch into the opening of the mounting with releasable anti-burst protection.

The pressure exerting belt, which surrounds the chest, is wearable also outside the vehicle. Pressing of the chest belt to the chest of the monitored person is provided by its flexible part.

The chest belt may be inserted under the clothing preferably represented by a shirt with electrodes and a monitor, preferably withdrawably fastened by snap fasteners with pulled on extensions with U-shaped profiles that hold the pressure exerting chest belt to the chest in the position above it, by snapping of the pressure exerting belt through the clothing into the U-shaped profiles.

The chest belt with a monitor, preferably fastened by snap fasteners, with openings for extensions mating with the U-shaped profiles or magnets that are placed there, preferably instead of the elastic strap used for fastening on the chest. The pressure exerting belt is snapped by means of magnets to the chest belt by the action of magnetic force through clothing, whereby an assembly is formed, and therefore the chest belt is fixed in the position defined by the pressure exerting belt. Similarly, when the pressure exerting belt passes through the U-shaped profile, it presses the chest belt to the chest. The Figure shows segments with a U-shaped profile that are fixed to the chest belt by fixing them in the openings that are provided in a common chest belt provided for fixing of an elastic belt placed around back of the monitored person. Alternatively, the U-shaped profile fixed in close proximity of the monitor and/or the case of the monitor is provided along the whole side turned to the pressure exerting belt by a groove into which the pressure exerting belt snaps.

The pressure exerting belt may snap into the extension with U-shaped profiles.

Preferable is the design of the halved pressure exerting belt that is pulled out of winches, preferably placed in the backrest, the halves of which are connectable by means of a buckle and a latch insertable into the buckle with a releasable lock against ejecting out. To the pressure exerting belt, is preferably fastened the chest belt, preferably by means of fastening of the belt, preferably formed by a bridge. Alternatively, a link passes through the pressure exerting belt to the monitor, which is preferably placed in the backrest, where from it passes the signal via a wireless link or a wired link.

The chest belt may be adapted for fastening to the pressure exerting belt by means of a bridge, which is insertable from the outside after opening of the clothing, preferably represented by a shirt, whereby its pressure of the chest belt to the chest of the monitored person is ensured. Pressure exerting belt may be fastened by a buckle.

One side of the pressure exerting belt may be provided with a fastening of the chest belt by a bridge in one place.

The chest belt may be inserted through the opened clothing, preferably represented by a shirt, at first in the direction of the arrow A.

The chest belt inserted under the clothing may be shifted in the direction of the arrow B into the center of the chest, with subsequent fastening up of the clothing, wherein the chest belt is now prepared for securing in the proper position by the pressure exerting belt, which acts by force in the direction towards the backrest and it is fixed on the other side of the backrest by inserting of the latch into the buckle.

The chest belt and the pressure exerting belt may be provided for fixation of the chest belt and of the pressure exerting belt instead of the U-shaped profile by magnets that are fastened both on the chest belt and on the pressure exerting belt so that so that they act on each other over the clothing and keep the chest belt in the required position on the chest.

Instead of the usual belt over chest the safety seat belts may be designed, as two pressure exerting belts at the levels of the waist and the chest to provide higher level of safety during simultaneous pressing of the chest belt to the chest by one of the pressure exerting belts, preferably of the upper belt, which serves simultaneously as a safety seat belt, whereas the second belt, preferably the lower one, serves preferably only as the safety one.

Preferable is the combination of the safety belt running obliquely over the chest with the pressure exerting belt running horizontally over the chest, for pressing of the chest belt, which preferably serves as the safety one to increase the safety.

The safety seat belt may run obliquely over the chest, which serves simultaneously for pressing of the chest belt inserted under the clothing secured in the position preferably by magnets placed on the chest belt with counterparts preferably on the external side of the clothing.

Preferable use of the fixation block is for pressing of the shortened chest belt to the body. The fixation block is connected permanently with the chest belt. After insertion of the belt under the T-shirt or under another piece of clothing, not openable on the front side, a pressure element is inserted into the recession formed by the fixation block through clothing which is exposed to pressure force exerted by the pressure exerting belt in the direction towards the chest of the monitored person, and thereby the electrodes are pressed to the skin. Because the fixation block is placed in the middle of the shortened chest belt, the monitor is placed off center.

It is preferable to fasten the pressure element, which is separable from the pressure exerting belt. The pressure exerting belt is connected with the adaptor for fastening of the adaptable pressure element. The adaptable pressure element is usable both when the fixation block is used and when the bridge with a spike is used.

Preferably, the pressure adaptor for horizontal or oblique pressure exerting belt.

Placing of the shortened chest belt under a shirt and passage of the spike of the bridge through an opening in the shirt is preferable.

Preferably, a template is used allowing turning of the monitored person on a seat of a transportation means when the horizontal pressure exerting belt is used. The shortened chest belt is provided with spikes with shoulders. The spikes mate into the cutouts of the template. The pressure force transferred to spikes of the pressure exerting belt acts on the template and via the spikes it acts on the chest belt.

It is preferable to use templates to allow making of a ECG record of the monitored person on a seat of a transportation means using the oblique pressure exerting belt.

It is preferable to use a template and spikes with a groove. Preferably, the spikes have groove mating with the inserted cutouts. In some place, preferably at the ends of the cutouts the cutouts are enlarged so that the spikes with grooves can be pushed through the template. After the following movement in the direction to the middle of the template a connection of the template, the chest belt and the pressure exerting belt is formed. Thereby, the chest belt with spikes is movable with regard to the pressure exerting belt with the template in the extent of the cutouts, wherein the length of the cutouts is chosen for the required extent of turning of the monitored person.

Preferable is the communication of the chest belt and of the pressure exerting belt by means of a bridge of the line “of the wired link of the monitor” through the pressure exerting belt, through the “connector of the pressure exerting belt-bridge”, through the bridge, through the “connector of the chest belt-bridge” and through the chest belt up to the “reduced monitor of the cardiac signals”. In this embodiment the monitor is fed by means of “a wired link of the monitor”. Therefore, preferably it does not comprise accumulator and/or circuits for wireless communication. The cardiac signals scanned by electrodes are processed in the monitor and transmitted further by means of a “wired link of the monitor”.

Preferable is fastening of the reduced monitor and of the “monitor supplement” to the chest belt. Mechanical connection is provided by means of the fastening elements, preferably snap fasteners. This solution allows power supply of the monitor both by means of the “wired link of the monitor” and the power supply from the “supplement of the monitor”. “The supplement of the monitor” comprises preferably an accumulator and communication circuits for wireless transmitting.

Preferable is the connection of the “supplement of the monitor” to the reduced monitor directly, by means of “the connector of the supplement”, which provides both mechanical and electric connection of both parts.

Preferable is the wireless recharging of the accumulator of the monitor in the working position by means of an induction charger. Such mechanical arrangements require the closest possible vicinity of the charger and the charged equipment. Preferably, the induction charger is integrated in the pressure belt or it can be fastened to it. The power for its operation is supplied by means of conductors running through the belt from the power distribution of the means.

The monitor may be connected by an external cable. This arrangement is used in case that the pressure exerting belt is not provided with a connector interconnecting with the bridge. The cable of the bridge is led from the “connector of the bridge external” into the “cable terminal” on the pressure exerting belt. From there it is led in the belt and/or on its surface to the power supply system of the means and/or to the controlling and displaying module, in case the wireless transmission between the monitor and the controlling a displaying module is not used.

Preferable is the multi-lead measuring of the ECG of the monitored persons sitting on a controlled chair of a transportation or other means In comparison to the previous figures, here is present the enlarged scanning of signals of the left arm, of the right arm, of the left leg and of the right leg by means of electrodes, preferably of the ones connected with the clips and/or wristbands, that press the monitoring electrodes to the wrists and ankles or to other parts of the limbs. Preferably, the clips are integrated in the seat, from which the monitored person controls the means which is controlled by the monitored person and from which they move for fastening into the working position. After releasing from the working position on a limb the input cable to the clip and/or to the strap is wound, preferably by a self-winding mechanism of the winch into the seat body and the clip moves into the clip holder. Preferably, the cables are routed over the auxiliary clips which reduce the pull of the winches and hold cables along the limbs. The signals of four electrodes for scanning of signals from limbs are brought into the monitor according to its position either on the belt or in the driver's seat and/or in another place, preferably in the dashboard of the means. If the monitor is placed on the shortened chest belt, the signals are brought into the monitor by conductors integrated into the pressure exerting belt that are routed into the bridge and further into the monitor. Alternatively, the signals are brought by conductors into the connectors on the shortened chest belt and/or directly into the monitor. To obtain multi-lead, up to 12-lead, ECG it is possible to use the shortened chest belt according to the PCT Application No. CZ17/000012, which chest belt comprises electrodes for scanning of the leads V1 to V6. The shortened chest belt is attachable to the pressure exerting belt by means of a bridge detachably or, preferably, rigidly, what allows in case of not using of the belt, once the driver disengages the pressure exerting belt that the pressure exerting belt together with the bridge and the chest belt rolls up by means of winches into the rest position on the seat side.

The above described configuration allows scanning of the one-lead ECG, either from the chest belt by using of the collecting electrodes and/or from the signals coming from the upper limbs. If a multi-lead ECG is required to the signals/leads LA, RA are added the signals of the lower limbs LL, RL and those from the chest belt V4, V5, V6. The 12-lead ECG is reached by using of the chest belt with electrodes in a curve or by using of the chest belt.

The input cables from the belts and/or the clips are preferably routed into the cable winches, so that only the required cable length is always unwound. Preferably, the clips and straps are provided with a connector for disconnecting.

Alternatively, the chest belt with the connected segment of electrodes usable for the 12-lead ECG. The monitor is placed on the belt and/or in the seat or in another place of the means. When it is placed on the belt signals of electrodes are brought to it by conductors routed through the belt and the segment of electrodes connected with the belt. Preferably, the monitor is powered by additional accumulator.

The signals of electrodes are brought to it by means of a connector of the pressure exerting belt and by conductors routed in the pressure exerting belt, when the monitor is placed off the belt.

Preferably, the monitor of thin design is implemented in the plate approximately of the size of a credit card. The monitor is easily portable and is suitable for instant measuring of the one-lead ECG in nearly any situation. In the monitor there are two contact areas that form electrodes. Further the monitor comprises a control unit of the monitor, a communication module, a control push-button, a SD card and an accumulator of the monitor. The communication module communicates with the operative controlling and displaying module, preferably with the mobile phone and further with a server.

The contact surfaces are attached to the chest and/or fingers are attached to the contact areas, on one contact area a finger of the left hand and on the other area a finger of the other hand. The integrated accumulator of the monitor is charged preferably over the charging contacts and/or inductively.

Preferably, the monitor implemented into the plate and provided with tipping plates with contact surfaces, whereby, after tipping a greater distance of electrodes formed by contact surfaces is obtained, what is more preferable for scanning of the ECG from the chest.

The case on the fastening strap serves for fastening of a monitor in the plate design and for its attaching to the chest of the monitored person. The monitor is inserted into the case, in which it is fixed and after attaching it to the chest of the monitored person, it is usable for the long-term measuring of the ECG.

An advantageous alternative of the monitor is the plate design for the middle time measuring, when the case is provided by additional accumulator, which over the charging contacts powers and charges. Alternatively, the additional accumulator is replaceable and chargeable over the cable or inductively. Preferably, the case is provided with a board with electronics and with the controlling push-buttons of the case

The power supply of the monitor is preferably of plate design in a case on a fastening strap by means of modular additional accumulators, wherein several of them can be placed on the fastening strap according to the required ECG scanning time. Additionally the modular accumulator also allows electrical connection for optimal power supply of the monitor. They are connected to the strap mechanically, preferably by snapping them or by a clip and/or they are connected with it by a connector and the strap is provided by conductors to connect all modular additional accumulators with a monitor. Alternatively, longer monitoring times can be obtained by mounting of an additional accumulator of larger capacity.

Preferably, the fastening strap is fastened to the case formed entirely or partially by a line of modular additional accumulators interconnected by stretchy modules for flexibility.

It is possible to use the case of the monitor for placing on the wristband. The case covers one contact area and at the same time it is connected with it conductively and with a conductor this area is connected with the contact area on the case side turned away from the hand. The contact area not covered by the case is in contact in the wrist area with the first hand of the monitored person. Once the monitored person touches the contact areas brought to the top of the case, it is possible to monitor ECG for a short time. For the middle time measuring the monitor is provided with a connector and it is directly or by means of the case connected with the glued electrode on the other arm or with the wristband on the other arm.

The monitor and the control module are connectable by means of a connector into one functional whole. The basic model of the monitor in a reduced form comprises only the block of the basic units, preferably one comprising a front end and a control unit. The block of supplementary units, which provides preferably the power supply by an accumulator and the communication by a communication block, is placed in the cooperating unit, where all optional functions are provided by the block of additional equipment comprising additional units and parts and a module of additional units. The cooperating unit to which the monitor is connected is preferably formed in particular by the controlling and displaying module, the operative controlling and displaying module or by the base of the monitor. This that the monitor comprises only the block has advantages in small dimensions, low price of the monitor and in the possibility to select differently equipped cooperating units. The monitor is connected for power and data supply to the cooperating unit by means of a connector, preferably by USB for the power and data supply. The contact surfaces or the electrodes are preferably connected by means of the connector of electrodes. Alternatively, the electrodes are connected by means of springs strip and contacts of the monitor or connector and or through the connector for cross-connecting and in case it is not the USB. Preferably, a second connector is used, preferably a USB, that serves as data and power supply for the module, preferably formed by a mobile phone, such as the common USB connector, which is preferably placed off the contact area of the connected monitor, so that it can be used also with the connected monitor. The block of additional equipment is both in the module and in the operative module, as well as in the base of the monitor replaceable for other equipment differently equipped as needed. Replacement is carried out by means of a fastening mechanism of the block. Movable self-sufficient model of the monitor is to be used for independent operation, including the power supply. Apart from the basic units it comprises additional equipment, preferably including the module of additional units. The self-sufficient model can be fastened by means of mounting elements, preferably formed by snap fasteners, to the auxiliary equipment, preferably comprising the chest belt, or the wristband or other auxiliary equipment preferably allowing not only fastening but also scanning of cardiac signals by means of contact areas placed on them.

By insertion of the basic model of the monitor into the base of the monitor, which base is provided with mounting elements, preferably represented by snap fasteners that allow to put the basic monitor on the auxiliary equipment, the basic model of the monitor fulfills the functions as a self-sufficient model.

The base of the monitor comprises electronics and recharging of the base, which controls the power supply from the additional accumulator or from an induction charger (not shown). Preferably, the self-sufficient model is produced provided with various additional equipment or it is a block of additional equipment with various equipment, which is replaceable, preferably connected by means of a connector and it is an advantage that due to fastening elements, preferably formed, the self-sufficient model of the monitor is replaceable on the pieces of the auxiliary equipment as it is actually needed.

The reduced monitor may be connected with the base comprising a supplement of the monitor. An externally placed additional accumulator allows long-term measuring of the ECG.

The reduced monitor may be connected with the contact surfaces to the mobile phone by means of a connector, wherein the monitor is adapted with form to this connection.

The reduced monitor may be connected with contact surfaces to the control module by means of a cable.

The monitor may be realized by connecting of the reduced monitor and of the supplement of the monitor suitable for the short time measuring of ECG.

The base of the monitor may be formed by a supplement of the monitor and by additional accumulator and by other board of electronics.

The monitor may be in the form of a card comprising contact areas, preferably placed in the opposite existing corners, preferably monitoring cardiac signals by attaching of fingers or chest, when the advantage of their greater distance by placing in corners is apparent. They are connected to the front end for analogue connecting of cardiac signals and transition to the digital form for further processing by digital controlling unit of the monitor, wherefrom they are transmitted to the input of the communication module for wireless transmission to the controlling and displaying module or operative controlling and displaying module or to a server, and/or they are stored to the memory medium, preferably to a SD card. The so provided monitor is adapted for independent scanning of cardiac signals, their processing and sending further to the cooperating unit or to storing in memory.

The view in the “D” direction shows the opposite side of the monitor, preferably with the opposite contact area placed above the contact area on the opposite side of the monitor for scanning from the wrist interconnected to the remote contact area in the opposite corner of the monitor, where a finger on the other hand is attached to the electrode of the contact area on the opposite side and preferably it presses the opposite electrode of the contact and this the wrist. The monitor comprises an accumulator, preferably it is placed to allow taking it out from a case of the accumulator. The monitor is controlled by the cooperating unit, preferably a module, and/or a server. In case it is controlled by means of the control elements, preferably represented by push-buttons, it is adapted for functioning as an independent unit, in which the processed cardiac signals are stored on the memory medium.

The monitor or the sensor of the desktop monitor may be in the form of a card fastened by the fastening element to the controlling and displaying module preferably formed by the mobile phone. The fastening element, preferably withdrawable one, is preferably formed by sticking means, e. g. by on both sides sticky tape or by Velcro hook-and-loop fastener or by a holder for fastening of the monitor preferably withdrawably fastened, preferably glued to the module shown in the “D” direction. The monitor sends the cardiac signals by means of a communication module further into the module, preferably formed by a mobile phone. Alternatively, the monitor is interconnected by a wired link through the connector of the module, preferably formed by the USB connector into the module. In this case, preferably, the monitor does not comprise the following parts: the accumulator of the monitor, the communication module, the control elements, and the SD card, which parts are placed in the module and used there.

The monitor without these parts is comprised of the sensor of the monitor and for functioning independently it has to be supplemented with the base of the monitor show in these figures.

The monitor may be fastened on the controlling and displaying operative module, preferably formed by a mobile phone on the wristband, or by a smart watch. Preferably, fastening of the monitor is realized withdrawably, preferably by a fastening element, preferably formed by an adhesive means, preferably by a double side adhesive tape or by the Velcro hook-and-loop fastener, or preferably, the monitor is adapted for plugging it into the holder, preferably placed withdrawably on the operative module.

Preferably, the monitor in the shape of a board exceeds the operative module, and so allows access to the contact area for touching with a finger of the other hand, which preferably exceeds the contact area on the reverse side of the board on the wrist of the first hand for scanning of cardiac signals, already preferably pressed also by action of the wristband.

The cross-connecting element may be put on the overlapping part of the monitor. To this cross-connecting element cardiac signals are brought from an electrode by a cable The electrode is preferably the electrode glued to the chest or the electrode on the wristband put on the other arm. The signals are brought to the contact areas of the monitor by means of a counter-contact. Preferably, the glued electrode is placed on the chest in the place recommended for scanning of signals of the other arm. The signals of the first arm are scanned by a contact area on the reverse side of the monitor, which is adjacent to the wrist of the first arm, which is not covered by the cross-connecting element. Alternatively, the cross-connecting element is connected also to the contact area and the reverse one and cardiac signals are brought from two electrodes, preferably placed on the chest, used for scanning of signals of the first and the other arm and are brought to the contact area and the reverse contact area.

Preferably, the cross-connecting element is provided with additional accumulator provided with a board with the charging electronics increasing capacity of the accumulators of the monitor, which accumulator is preferably replaceable. In this case the accumulator of the monitor preferably forms a bridging accumulator providing power supply during the time period of replacing of the additional accumulator.

Preferably, the monitor is placed in the fastening means, preferably formed by a holder, into which it is preferably inserted. The fastening element is adapted for fastening of the remote contact area, which is placed remotely from the monitor, and so the remote contact areas for scanning of cardiac signals by attaching to the chest, and to connect the remote contact areas to the nearer contact area on the monitor, which area is covered by it. Preferably, it is realized by a holder with a fastening element, preferably formed by a plastic extension, preferably withdrawably fastened by a hinge on a holder, by means of which it is adapted for tilting it over the holder. The contact area on the fastening element is preferably interconnected with the contact area on the monitor, preferably by means of a wired interconnection and the opposite contact on the holder or the connector during inserting of the monitor into the holder or the connector when the monitor is inserted into the holder. The connecting members are adapted for connecting of the stretchy strap for permanent connection of the monitor to the chest for permanent monitoring.

Preferably, the fastening means are formed by a holder with an extension member, where the inserted monitor is connected by a connector.

To increase capacity of the accumulator an additional accumulator may be connected, which is connected with the monitor by means of cross-connecting areas on the holder and the monitor that are attached to each other when the monitor is inserted.

Preferably, the additional accumulator is withdrawably fastened on the chest belt with contact areas connected to the monitor, which is by a connector connected to the additional accumulator.

The monitor or the sensor of the monitor is preferably placed extractably in the operative module, which is connected by a connector to it, where the extracting is carried out preferably by taking it out, or by opening the door and extracting.

The sensor of the monitor may be connected with the base by means of inserting it into the holder of the sensor, which is an extension of the base, where a monitor comes into existence by interconnecting with the connector.

The sensor of the monitor may be connected with the base of the monitor preferably by insertion on spikes and the connector.

The sensor of the monitor may be fastened to the base of the monitor, which base is connected to the shortened chest belt with contact surfaces for attaching to the chest.

The sensor of the monitor is connected with the base of the monitor by means of the connector of the sensor of the monitor and the connector of the base.

Withdrawably is connected the elastic strap making permanent monitoring possible by fastening it to the chest.

Preferably, the sensor of the monitor is fastened by a fastening element to the module, which is preferably formed by a mobile phone together with a holder provided with a connector, into which the sensor is inserted. From the connector a cable is connecting to the module for communication. The contact surfaces are placed on the sensor and/or on the module.

Preferably, the monitor comprises a sensor of the monitor connected with the base of the monitor connected by a cable to the connector into the module or which is communicating by means of a communication module wirelessly.

Preferably, the monitor communicates by wires or wirelessly with the operative module or for displaying and/or controlling. Preferably, they communicate with each other and pass data about the processed cardiac signals and/or from a phone voice call. The modules transmit data about the processed cardiac signals further on into a server provided with access rights for remote users.

Preferably, the monitor is interconnected with the controlling and displaying operative module, preferably formed by a mobile phone or by a smart watch, wirelessly or by wires by means of a connector, preferably by a USB for data transfer transmitting health data about at least one of the following group: ECG, pulse values and/or curves of heart rate with the limits of the regular heart rate, arrhythmia, variability. The monitor is mounted on the base of the monitor, preferably withdrawably, and it is connected with the electrode of the first arm, preferably to the bottom part of the base, which sits on the wrist. The electrode of the other arm is placed preferably on the top part of the monitor for touching it with a finger or a wrist, for what it is provided with sufficient surface area. In case of connecting of the monitor by means of a connector with the operative controlling module, the monitor preferably is not provided with an accumulator and a wireless communication unit, wherein it is powered from the accumulator of the operative module and it communicates by means of a connector. Preferably, to the monitor is by means of a connector, optionally over the cross-connecting cable of the additional accumulator connected an additional accumulator, which is preferably fastened to the extended base and/or to a strap on the wrist. Preferably, into the connector on the accumulator is connected a cable for connecting to the electrodes on the other arm, which is placed on the wristband on the other arm or which is glued on the chest, or to the electrode of the first arm and the electrode of the other arm on the glued electrodes on the chest or on the chest belt.

In case, the accumulator is not used, the connector cable is inserted into the connector of the monitor.

Extracting of the operative module from the base may be carried out by its shifting, and by the following tilting. A spring holds the operative module pressed to the connector in the inserted position. In the extracted position, the operative module is adapted for holding of phone calls over the mobile operator's network.

The accumulator and/or the monitor may be placed withdrawably in the base, preferably by insertion into the shelves of the base.

Preferably, a reducer is used, which reducer is inserted into the connector and is provided with the first contacts on the contact surface with monitor that are in contact with the second contacts placed on the monitor in the plugged state of the operative module, what allows easier insertion than when a connector would be inserted.

Preferably, the monitor may be repositioned together with the accumulator to the chest belt by means of the base, into which they are inserted. The base is placed on the belt preferably by means of snap fasteners. The monitor is electrically and mechanically interconnected withdrawably with the base and is connected to the electrodes on the chest belt.

The base may be fastened on the chest belt with shelves for inserting of an accumulator and of the monitor that are connected to the electrodes of the chest belt directly.

Preferably, the monitor may be extractably inserted into the monitor, which is represented preferably by extractable smart watch, preferably also with an inserted accumulator. The principles are the same as in case of the monitor represented by a mobile phone.

Preferably, the smart watch presses the base to the wrist by action of the strap tightened to the wrist. The base comprises a fastening adjusting, which is preferably formed by a protrusion, which prevents taking off of the base from the space under the smart watch, whereby the monitor is fastened with the base. The communication is wireless or by means of a connector. The monitor is connected permanently on the base, or withdrawably by means of a shelf other than what are the shelves for the smart watch.

The extensible arresting by means of a retractable mechanism is preferable for adjusting of the base length for various modules for versatility.

Preferably, the monitor is fastened to the operative module by means of a plug-in, fastening mechanism, preferably formed by some embracing housing of the module or put-on spikes. Optionally an accumulator is put on the monitor.

Preferably, the monitor is placed in the operative module permanently or extractably after opening of the door or of a cover and is extractable from the connector or it is dismountable. The extractable monitors are portable to the chest belt. The monitors are provided with touchable electrodes for touching by the first hand and/or the second hand and with an additional accumulator by means of a connector. Preferably, the module is fastened on a wrist strap placed on the wrist, preferably withdrawably, preferably by means of a base.

Preferably, the control module is of smaller dimensions. It is fastened on the wrist by means of the base with a connected monitor, the accumulator, the electrode on the other arm on a strap, where the monitor and/or control module and/or displaying module communicates over a mobile network, preferably over a formed network of a mobile operator and/or over a WiFi network with a server, preferably with the server of a surveillance center and with the connected participants for transmissions of health data from the monitor of cardiac signals and/or from the block of the monitor with the health data, which data are formed preferably by at least one piece of data from the following group of instruments: a blood pressure gauge, a blood oxygen sensor, a body weight sensor, a thermometer, a pedometer, a breath monitor, a physical activity monitor, which instrument reports an alarm when the person under surveillance is not active, a monitor of a fall, a health condition monitor.

The monitor for scanning of the one-lead ECG is placed on the wrist and scans by means of electrodes.

For a short-time scanning, preferably electrodes of the monitor are used, wherein one of the electrodes is placed on the bottom side of the fastening board, on which the monitor is placed or on the bottom side of the monitor, for contact with the wrist of the left hand which belongs to the system of electrodes of the left hand and the second electrode is placed on the top part of the monitor and serves for contact with fingers or the wrist of the right hand belonging to the system of electrodes of the right hand.

For a contact with the wrist by the system of electrodes of the first hand preferably formed by electrodes placed on the bottom side of the monitor, the fastening board is provided in the place of the electrodes by an opening, allowing contact of electrodes with the wrist.

To avoid the necessity to hold the right hand on the system of internal electrodes of the monitor, in particular for the long-term monitoring, preferably, the signals of the right hand are scanned by the system of electrodes on the other arm, instead of touching the monitor with fingers. Preferably they are placed on the right hand on the wrist, they are fastened preferably by tapes on the wrist or by clips, or they are fastened preferably by tapes on arms or they are fastened by gluing on the right shoulder or on the right side of the chest.

For the signals of the left hand instead of the system of the internal electrodes of the monitor, alternatively, the system of the glued external electrodes is used on the on the left shoulder or on the left side of the chest or they are fastened by a strap on the arm.

For a short-time scanning from the chest, which gives less disturbed signal than the scanning from arms, the monitor is adapted for scanning from the plate with electrodes by attaching them to the chest, wherein, they are connected to the monitor by a cable by means of a connector, or the monitor is adapted for repositioning from the wrist to the board for fastening and by means of a connector it may be connected to the electrodes. For the long-term scanning from the chest the chest belt with electrodes is connected with a monitor either by a cable or the monitor fastened to the belt is moved from the wrist to the chest belt by some mounting elements, and preferably, it is connected wirelessly with the operative module, or chest belt with its own monitor is used.

For the long-term scanning from arms during movement, preferably, the ECG curve is dampened in the software by filtering of the disturbing signals, to remove interference caused by muscle activity, with the result that not only elimination of interference takes place but also details of the ECG curve are lost, such as for example of the “P” wave or of the fibrillation waves. If necessary, the detailed ECG curves are scanned at rest and the ECG curve is filtered less or not at all. So, the level of filtration, preferably the ECG curve, is adapted to various levels of damping in response of various level of movement, for example for resting, walking, running, and the maximal damping is used for the extreme movement, wherein, preferably, the levels of damping are switched by a controlling element placed on the monitor or off the monitor, preferably in the operative module, as required. For a less interfered signal also during movement allowing lower level of filtration, and therefore, m more detailed ECG curve. Preferably, the monitor for scanning from the chest by the chest belt is used. Because scanning by using the chest belt is not so comfortable as the scanning from the wrist, for common monitoring, at first, the more comfortable scanning from arms, wrists, or fingers is used at first wherein, preferably a higher level of filtering by means of a software allowing ECG scanning during movement or of fingers is used at first. When deviations from the standard ECG are found, whereby, the need to provide ECG of higher quality is discovered, the ECG scanning is carried out, at best from the chest at rest and at a lower level of damping by means of a software.

Therefore, preferably for the orientation monitoring a more dampened ECG is used, what allows monitoring during movement and for comfort monitoring by means of the signals from the hands, and for the detailed monitoring, for example when deviations from the standard ECG were found, the less dampened or non-dampened signal of ECG is used, preferably the one scanned from the chest.

The scanning of the one-lead ECG by the monitor is preferable for the preliminary diagnoses, and the multi-lead ECG is used for the more detailed diagnoses if necessary, wherein, preferably, the universal monitor adapted both for the one-lead scanning and for the multi-lead scanning is used, wherein, the multi-lead scanning is used for example when deviations from the standard are found in the preliminary diagnoses.

Preferably, for the sake of simplicity the one-lead scanning is used with the universal monitor, which monitor is adapted also for scanning of the multi-lead ECG, or for the scanning of the multi-lead ECG is used another monitor, additionally to the monitor for the one-lead scanning.

For an easy replacement or for repositioning of the monitor, preferably mounting elements are used.

For the multi-lead scanning by two electrodes from the system of electrodes of the left hand or of electrodes of the monitor for the left hand and the system of electrodes of the right hand, preferably used already for the one-lead scanning, the system of electrodes of the left leg and the system of electrodes of the right leg are added, wherein the electrodes are fastened by tapes on legs, or they are fastened by clips or thy are glued above the legs interconnected by cables to the monitor, which monitor is preferably placed on the wrist, and this is used for measuring of up to 6-lead ECG, when to the measured values for the leads I a II are calculated the values for the lead III and the leads aVR, aVL, aVF.

For scanning of the from 7-lead to 12-lead ECG preferably a plate with electrodes for V1 to V6 is added, wherein, preferably, they are connected by a cable to the monitor, which monitor is preferably placed on the wrist. In case of one electrode on the plate, optionally it is possible to monitor one of the leads V1 to V6 according to the place to which the plate is put or all leads V1 to V6 are gradually moved.

In case of two electrodes on the plate, it is possible to monitor optionally 2 leads from the V1 to V6, and therefore, when repositioning is carried out three times, it is possible to scan all leads V1 to V6.

In case of 6 electrodes on the plate, it is possible to monitor leads V1 to V6 at once. Preferably, the plate is of an elastic material to be able to adapt to the chest shape and it is to be used by holding it on the chest for the short-time scanning. For the long-term scanning, preferably, the plate is mounted permanently to the embracing strap for permanent fastening it to the chest, whereby a chest belt is formed.

Alternatively, the monitor is preferably placed directly on the plate, where it is placed preferably for the multi-lead scanning from the wrist, and a cable transmits the signals from electrodes to it, or preferably it may be placed or repositioned to the trouser belt and it comprises also a buckle used for fastening.

In case, the monitor is to be used also for the one-lead scanning, it is fastened on the chest belt for the long-term scanning, it is connected to the system of electrodes of the first hand and the system of electrodes of the other hand is connected by a cable to the monitor. For the multi-lead scanning, optionally the system of the right leg electrodes and the system of the left leg electrodes for the lead III and the leads aVF, aVL, aVR a are added as needed, optionally the electrodes for V1 to V6, preferably on the chest belt. In case, the one-lead monitor is used for the multi-lead ECG, the monitor is replace by a multi-lead monitor, preferably by fastening it by means of some mounting element.

Preferably, the electrodes fastened to the chest are glued by some gel to a target or they are fastened by suckers. The electrodes on arms or on wrists are preferably fastened by tapes or by clips or they are fastened on the monitor.

Preferably, in the beginning of scanning, for simplicity, the scanning is used for preliminary diagnoses using the one-lead ECG and grater damping of the ECG by intensive filtering to allow scanning to a monitor from hands during movement, and if deviations from the standard are found a multi-lead ECG at rest is scanned, without any damping by filtering, or the one-lead or the multi-lead ECG scans from the damping during movement and after end of damping at rest to obtain greater details in the display.

Another advantage is that for the preliminary diagnoses the heartbeat curve is monitored and evaluated, preferably with the limit curves of the regular heartbeat, without damping, or with only slight damping of signals by means of software, and if deviations from the standard are found the one-lead or the multi-lead ECG is scanned as required in movement using greater damping and/or at rest without damping.

### BRIEF DESCRIPTION OF THE DRAWINGS

FIG. 1 shows a monitor repositioned to a chest belt fastened by means of mounting elements;

FIG. 1 Detail 1 shows schematically the system of electrodes in an enlargement;

FIG. 1 Detail 2 shows an example of the monitor repositioned to the chest belt for the 12-lead ECG;

preferably shows in an enlargement an example of the monitor repositioned to the chest belt for the 12-lead ECG;

FIG. 1 Detail 4 shows using of the monitor on the chest belt for the one-lead ECG, wherein, if necessary the monitor is repositioned to belt adapted for scanning of the multi-lead ECG by repositioning of the monitors, preferably to the positions 1 to 12;

FIG. 1 Detail 5—on the multi-lead belt; one position is for scanning of the less leads having ECG, for example of the one-lead, is used as the common scanning, and, if necessary, by repositioning of the monitor into other positions in the field there are 180 monitoring positions, 1 to 12 scan the multi-lead ECG, for example up to the 12-lead one;

FIG. 2 shows, for example in case of the one-lead scanning, the handling during repositioning of the monitor to the individual positions, for example to the positions 1 to 12, for providing of the 12-lead ECG;

FIG. 3 shows the preferably detachable placing of the printer underneath the notebook for the purposes of the ECG for printing of the ECG reports directly from the notebook;

FIG. 3 Detail 1 shows a disconnected printer, connectible to the notebook by means of a mounting mechanism of the printer and the printer connector,

FIG. 3 Detail 2 shows a closed notebook with a connected printer and a detachable loop-strap for easy carrying;

FIG. 3 Detail 3 shows forming of a set of a notebook with a printer by means of the mounting mechanism, preferably formed by a universal mounting mechanism;

FIG. 4 shows an example how to use a belt provided with a monitor in the physician's waiting room or in an ambulance in a hospital;

FIG. 5 shows communication between a notebook and a monitor, which monitor is preferably connected to the notebook connector, preferably represented by a USB, wherein via the USB it communicates and receives power, so that preferably in the monitor, there is neither an accumulator nor a wireless communication module;

FIG. 6 shows a chest belt with switchable electrodes and a monitor transmitting data wirelessly and/or via wires;

FIG. 7 shows a design of the chest belt in a variant without any monitor, wherein the system of electrodes on the belt and the system of external electrodes are connected directly with an evaluation unit or a desktop ECG;

FIG. 8 shows a chest belt provided with a monitor, electrodes for scanning of the leads V1, V2, and V3, and with a cross-connecting bridge;

FIG. 9 shows a chest belt with the switchable probes for the leads V1 to V3, which is enhanced by adding a system of external electrodes for the leads RA, LA, RL, LL to measure a 9-lead ECG;

FIG. 10 shows a chest belt with a monitor, a selector switch of the V-leads and with an additional board for scanning also via the probes V1 to V3;

FIG. 10 Detail 1 shows a chest belt for scanning of up to 10-lead ECG with an additional board and with a monitor, to which the leads RA, LA, RL, and LL are brought directly;

FIG. 10 Detail 2 shows a chest belt for scanning of up to 12-lead ECG, wherein signals of the probes V1, V2, V3 are brought in over the electrodes placed on independent springs;

FIG. 11 shows a system of the monitor provided with a chest belt and collecting electrodes;

FIG. 12 shows a system for scanning of the multi-lead ECG using a foldable scale;

FIG. 13 shows an attachable monitor using marking of the position;

FIG. 14 shows attachable sensor with an auxiliary rail;

FIG. 15 shows an attachable sensor with a guiding rail and a bridge;

FIG. 16 shows using of a fold away scale;

FIG. 17 shows a belt with a possibility to select the V leads;

FIG. 18 shows using of the block belt and a segment of electrodes;

FIG. 18 Detail 1 shows a segment with electrodes;

FIG. 18 Detail 2 shows a strap with electrodes

FIG. 19 shows using of a snap-on segment of electrodes and of a selector of leads;

FIG. 19 Detail 1 shows a segment of electrodes;

FIG. 20 shows using of a diminished system of electrodes;

FIG. 21 shows using of braces in the system of electrodes;

FIG. 22 shows the positions for placing of electrodes for scanning of the leads V1 to V6;

FIG. 23 shows a chest belt with a monitor placed under the clothing and a pressure exerting belt;

FIG. 23 Detail 1 shows a pressure exerting belt with one winch in the backrest;

FIG. 23 Detail 2 shows a pressure exerting belt with a stretchy part wearable also off the vehicle;

FIG. 23 Detail 3 shows a chest belt with electrodes and a inserted under the clothing;

FIG. 23 Detail 4 shows a chest belt with a monitor preferably fastened by snap fasteners;

FIG. 23 Detail 5 shows a pressure exerting belt, wherein the correct position is ensured by U-shaped profiles;

FIG. 23 Detail 6 shows a halved pressure exerting belt, wherein the halves are connectable by means of a buckle and a latch;

FIG. 23 Detail 7 shows a chest belt adapted for fastening to the pressure exerting belt by means of a bridge;

FIG. 23 Detail 8 shows a pressure exerting belt with fastening of the chest belt by a bridge in one place;

FIG. 23 Detail 9 shows inserting of a chest belt under the unbuttoned clothing;

FIG. 23 Detail 10 shows shifting of a chest belt already inserted under the clothing in the direction of the arrow B into the center of the chest, with subsequent buttoning of the clothing;

FIG. 23 Detail 11 shows a chest belt and a pressure exerting belt provided with magnets for fixing of their connection;

FIG. 23 Detail 12 shows an implementation of the safety belts in the form of a sash placed obliquely over the chest by means of two pressure exerting belts;

FIG. 23 Detail 13 shows a combination of a safety belt placed obliquely over the chest and a pressure exerting belt;

FIG. 23 Detail 14 shows a safety belt placed obliquely over the chest, which serves simultaneously also for pressing of the chest belt inserted under the clothing;

FIG. 24 shows using of a fixation block for pressing of the chest belt to the body;

FIG. 24 Detail 1 shows detachable fastening of the pressure exerting element;

FIG. 24 Detail 2 shows adaptors for the horizontal and the oblique pressure exerting belts.

FIG. 25 shows placing of a shortened chest belt under a shirt and passage of a bridge spike through an opening in the shirt;

FIG. 25 Detail 1 shows using of a template for allowing of a turning of the monitored person sitting in a seat of a means when the horizontal pressure exerting belt is used;

FIG. 25 Detail 2 shows using of a template for allowing of turning of the monitored person sitting in a seat of a means when the oblique pressure exerting belt is used;

FIG. 26 shows using of a template and of spikes with a groove;

FIG. 27 shows connection of the chest belt and the pressure exerting belt by means of a bridge with an input of the cable into the monitor;

FIG. 28 shows fastening of the reduced monitor and of its supplement to the chest belt;

FIG. 29 shows connection of the supplement to the reduced monitor,

FIG. 30 shows wireless recharging of the monitor accumulator in the working position;

FIG. 31 shows interconnection of the monitor by an external cable;

FIG. 32 shows a multi-lead ECG scanning of the monitored persons;

FIG. 33 shows a monitor of thin design.

FIG. 34 shows a monitor of thin design with hinged plates;

FIG. 35 shows a monitor of thin design fastened in a case on the fastening strap;

FIG. 36 shows a monitor of thin design in a case with a board of electronics and an additional accumulator; FIG. 37 shows an additional accumulator on a fastening strap;

FIG. 38 shows an additional accumulator integrated in the fastening strap;

FIG. 39 shows a monitor in a case to be used on a wristband;

FIG. 40 shows a block diagram of a reduced monitor for connecting to a mobile phone;

FIG. 41 shows a block diagram of a reduced monitor for connecting to the base of the monitor;

FIG. 42 shows connecting of a reduced monitor to a mobile phone by means of a connector;

FIG. 43 shows connecting of a reduced monitor to a mobile phone by means of a cable;

FIG. 44 shows a reduced monitor of thin design connected to the supplement of the monitor;

FIG. 45 shows a reduced monitor with a supplement of the monitor, a board with electronics, and with an additional accumulator,

FIG. 46 shows a monitor in the shape of a card;

FIG. 47 shows fastening of the monitor to the controlling and displaying module;

FIG. 48 shows fastening of the monitor to a module represented by a mobile phone or by a smart watch;

FIG. 49 shows a monitor with a cross-connecting element;

FIG. 50 shows a cross-connecting element with an additional accumulator,

FIG. 51 shows a monitor in a fastening means;

FIG. 52 shows an example of a fastening means;

FIG. 53 shows a connection of the additional accumulator;

FIG. 54 shows a fastening of the additional accumulator to the chest belt;

FIG. 55 shows placing of the monitor or the sensor in the controlling and displaying module;

FIG. 56 shows connection of the sensor of the monitor with the base of the monitor;

FIG. 57 shows connection of the sensor of the monitor and the base by means of spikes;

FIG. 58 shows using of a shortened belt for the permanent monitoring;

FIG. 59 shows a configuration of the sensor of the monitor, of the controlling and displaying module, and of a holder, with contact surfaces on the sensor,

FIG. 60 shows a configuration of the sensor of the monitor with the base of the monitor;

FIG. 61 shows communication of the monitor, of the controlling and displaying module and of the server;

FIG. 62 shows a monitor connected with the operative module by wires or wirelessly;

FIG. 62 Detail 1 shows a base for the monitor and an accumulator with shelves;

FIG. 62 Detail 2 shows a connector connection of the controlling module with the monitor,

FIG. 63 shows placing of the monitor and the accumulator on the chest belt by means of the base;

FIG. 63 Detail 1 shows the base with the shelves placed on the chest belt;

FIG. 64 shows a monitor extractably placed on the controlling module represented by a smart watch;

FIG. 65 shows fastening of the base for monitor to the smart watch;

FIG. 65 Detail 1 shows extractable adjusting of the base for universal connectivity to various models of the smart watch;

FIG. 66 shows a put-on fastening mechanism for fastening of the monitor to the controlling module;

FIG. 67 shows variants of placing of the monitor in the controlling module;

FIG. 68 shows fastening of the monitor and of the controlling module on an arm and wireless transmission of the health data;

FIG. 69 shows variants of placing of electrodes for scanning of the one-lead ECG by means of the monitor,

FIG. 69 Detail 1 shows using of electrodes in the short-time scanning of the ECG with the electrode of the left hand;

FIG. 69 Detail 2 shows use of electrodes in the short-time scanning of the ECG with the electrode of the left hand on the monitor, wherein this electrode is designed mechanically so that it passes through the board;

FIG. 70 shows possibilities of configuration of the electrodes to obtain a multi-lead ECG by means of the monitor or the monitors.

### EXAMPLES OF EMBODIMENTS OF THE INVENTION

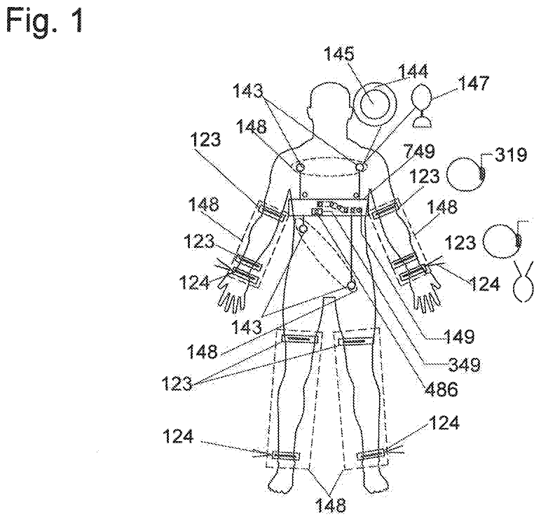

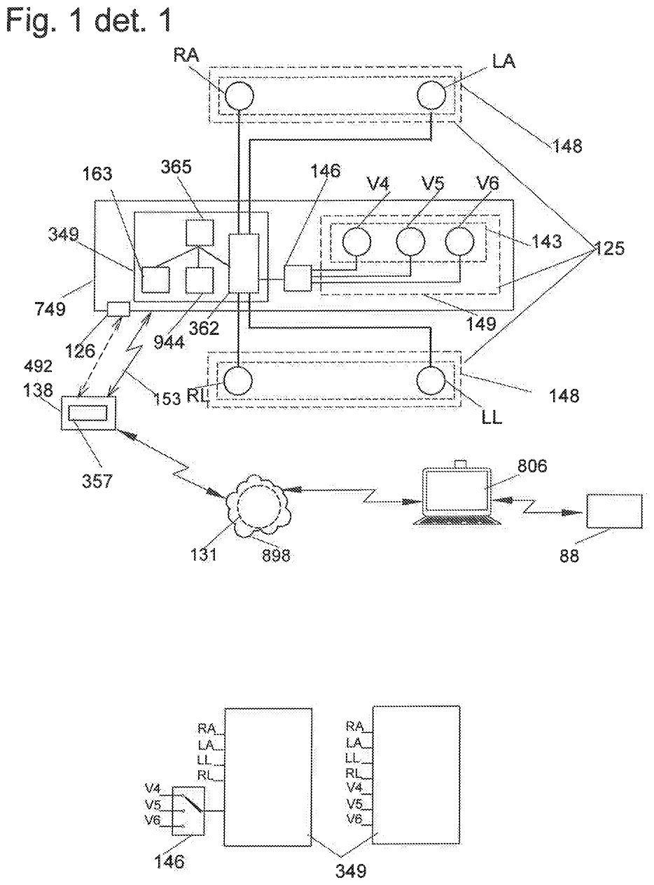

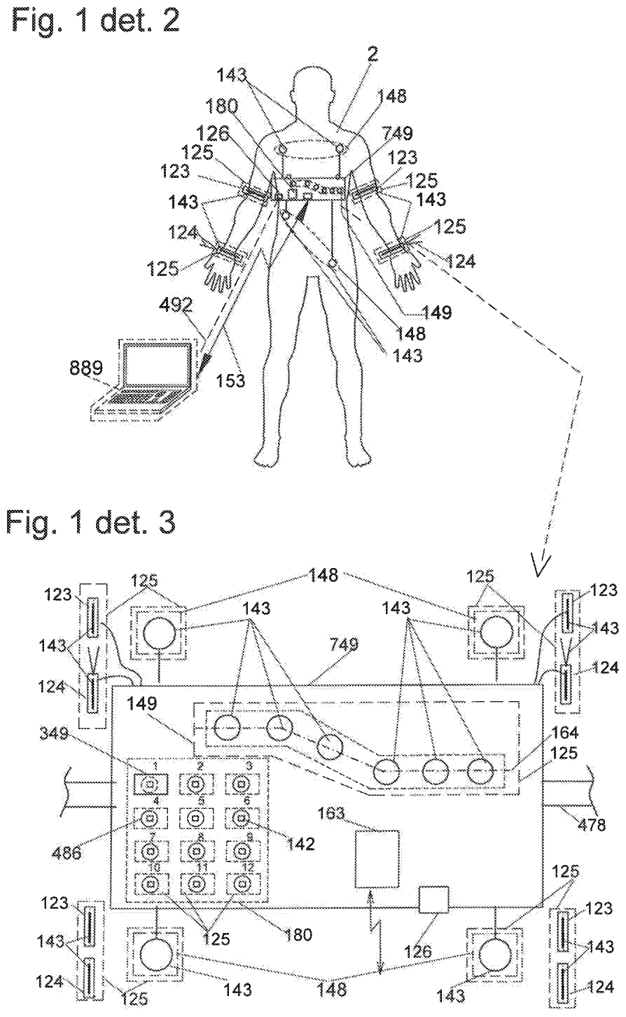

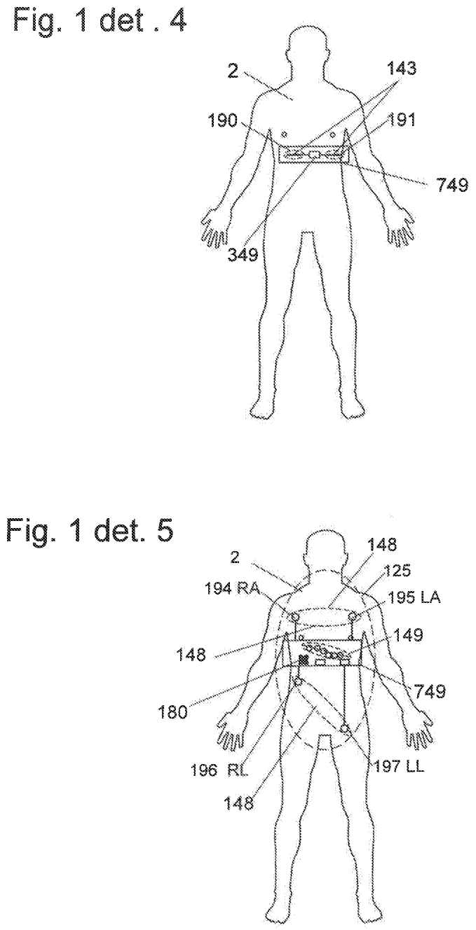

FIG. 1 shows a monitor 349 on a wide chest belt 749, fastened, preferably withdrawably, by means of mounting elements 486 connected to the electrodes 143, where a part 143 of electrodes, in this example six of them, is preferably placed on the chest belt and forms the system 149 of electrodes on the belt 749 and the part 143 of electrodes, in this example four of them, is preferably fastened on the body of the monitored person and forms system 148 of external electrodes either by adhesive target 144, preferably withdrawably by conductive gel 145 or by suckers or by fastening straps 123 or on arms and things or by wristbands or by clips 124 on the wrists and ankles and is connected by wires and by their communication a system of electrodes 125 comes into existence, which is easier to install on the body of the monitored person than to install individual electrodes. The mentioned layout of electrodes is an example for the 12-lead ECG, a for various applications and the number of leads may be number of electrodes adapted, as well as their layout. In case of indication of a reference to the system of electrodes 148, 149 or 125 without other details, the system shown in FIG. 1 is meant.

Detail 1 shows schematically an example of the narrow chest belt 749 in enlarged scale with three electrodes 143 in the system of electrodes 149 on the chest belt 749 and four electrodes 143 in the system of external electrodes 148. The monitor 349 is in this example for three-lead application expandable up to a nine-lead one.

For the three-lead application electrodes RA, LA, LL, RL, V4 are connected to the monitor 349 and by output ECG curves for leads I, II, V4 are measured. By connecting of the electrodes, preferably by a selector switch 146 of electrodes to the electrode V5 a then V6 the ECG record is enlarged by including the curves for the leads V5, V6, that are subsequently measured and added to the record for application of the measured 5 leads. By calculation of the leads III, aVR, aVL, aVF and 7-lead to 9-lead application is obtained. The output signal is transmitted by a wired link 492 through the connector 126 of the chest belt 749 or by wireless link 153 from the monitor 349 towards the evaluation unit 138, preferably formed by the controlling and displaying module 358 or the operative controlling and displaying module 357 or by a mobile phone 100 or by a tablet or by a notebook 173 or by a PC 889, from which it is preferably transmitted into the server 806 by means of WiFi network 131 or a mobile operator's network or via Internet and from there to the remote participants 88. Wirelessly the transmission is realized from the monitor 349 by means of a wireless module 163, which is alternatively, adapted for transmission over the WiFi network 131 or a mobile operator's network directly into the server 806. The specified example of the system 125 of electrodes is preferably the seven-electrode one, of which with three electrodes on the chest belt 749. Preferably, this system is usable for a Holter with recording to an extractable memory medium 964 that is portable to the to the evaluation unit 138 for the downloaded record. Instead of the selector switch a monitor for measuring also of the leads V5, V6 is usable alternatively. Preferably, the selector switch 146 is formed by a mechanical selector switch provided with three positions or with a portable jack or a DIP selector switch or with an electronic selector switch.

Detail 2 shows an example of the monitor 349 repositioned to the chest belt 749 or placed on it for the 12-lead ECG

Detail 3 shows in an enlargement six of electrodes 144 that are placed in the system of electrodes, preferably on a wide chest belt 749. They are loosely attached to the chest or fastened by the encircling pressing elastic band 478, which allows layout of electrodes on the axis 164 that is optimal for scanning of the 12-lead ECG. Other four electrodes 143 in the system 148 of external electrodes are connected by wires.

The monitor 349 is placed, preferably withdrawably, on the belt 749 in the field of positions and is adapted for scanning of up to 12-lead ECG at once, or such monitor 349 is used that is adapted for scanning of only a part of the leads. In case of adaptation for six leads the monitor 349 is moved after scanning of the six leads in the position 1 to the position 2 for scanning of another six leads, i.e. of twelve leads together. In case of the adaptation for four leads it is moved three times to the positions 1, 2, 3 for scanning of twelve leads. Similarly, in case of using of monitors for scanning of three leads it is moved four times to the positions 1 to 4, in case of two leads six times to the positions 1 to 6, in case of one lead twelve times. In case of each repositioning, the monitor 349 is fastened over the mounting elements 486, preferably with connectors 142 for connecting to the respective electrodes.

An advantage of the repositioning of the monitor into the connectors 142 of the chest belt is the saving for a cheaper monitor for example for only four to six leads. In case that the belt 749 is to be used for scanning of the same number of leads as the monitor 349, preferably, from the array 180 is used only the connector and the rest is not necessary to install or the monitor 349 is connected to the belt permanently.

The system 125 of electrodes with six electrodes 143 within the system 149 of electrodes on the chest belt 749 with up to four electrodes 143 in the system 148 of external electrodes existing off the chest belt, connected by wires to the belt is preferable in that it is easy to install the system for ECG scanning and the installation as a whole, is preferably in comparison to individual electrodes connected by wires to the monitor 349 or to the evaluation unit 138. Moreover, the transmission from the monitor 349 to the unit 138 directly from the electrodes on the belt or from the electrodes connected to it via the connector 181 of electrodes, see FIG. 1 allows an easier installing than by connecting the evaluation unit 138 directly to individual electrodes by wires. The monitor 349 placed on the belt 749 is connected by a wire link 492 or a wireless link 153 to the evaluation unit 138, preferably formed by a notebook 173 or by a PC 889 or by a mobile phone 100. The 12-lead system 125 of electrodes connected directly by wires, without a monitor to the evaluation unit 138 is used preferably for providing an ECG on the unit 138 preferably formed by a desktop ECG instrument, comprising a front end or by a notebook 173 or by a PC 889, which are supplemented by circuits of the front end that are placed preferably in a dongle. In this way the evaluation unit 138 is adapted for data processing of cardiac signals and for displaying or storing them in a memory or for printing of them in the usual form of an ECG, preferably together with a diagnosis and the ECG parameters. Preferably, by this, the 12-lead system will replace the common ECG, either by interconnecting of the 12-lead system 125 to the desktop ECG apparatus or to a notebook 173 or PC 889.

The 12-lead system 125 is preferable for monitoring of events that can be uncovered only with difficulty on an ECG with less leads, for example the heart attack or the sites of occurrence of ventricular extra systoles or bigeminies. During a long-term scanning, it is preferable to display on the display of the evaluation unit 138, preferably of the module 358, 357, information about cardiac activity. preferably arrhythmia or occurrence of a ventricular extra systole with a number per time unit for evaluation of the cardiac activity, preferably for athletes and monitored persons with heart disease, so that these persons can modify their regime on a continuous basis.

Detail 4 shows using of the monitor 349 on the narrow chest belt 749 for the one-lead ECG, where preferably the electrodes 349 are formed as the collecting electrodes 139, which are longer and are placed in another place than the common ECG electrodes 143, and they scan the aggregate signal for more leads ECG, for example for leads I, II, a V1-V6 and for other ones, whereby, they allow a summary overview about all leads in a simple way from the one-lead in a narrow chest belt 749, and thus the assessment in a simple way of the degree of imbalances of the ST sections that are important in the evaluation of the heart attack or angina pectoris, in particular under load, for what would otherwise be necessary the more complicated, for example the 12-lead ECG. If necessary, the monitor 349 moved to the belt 749, which is adapted for scanning of the multi-lead ECG, by repositioning of the monitors in within the field positions into various positions, and/or into only one position for the required number of leads as it is shown in the Detail 5. Repositioning of the monitor 349 from a belt for a lower number of leads to a belt with a higher number of leads and to other positions allows costs savings in comparison to a more expensive monitor 349, which would allow scanning with more, for example with 12 leads, without repositioning and which would be more complicated than the one-lead monitor 349, and which would be moreover used only sometimes, so that the costs spent for it would not be fully used. And moreover, the multi-lead belt is larger, so that it is more comfortable to wear the smaller belt with a lower number of leads when the greater number of leads is not necessary. The more leads are used in the monitor 349, the simpler is the handling, with a lower number of repositioning, but it is at the expense of higher costs. Scanning without repositioning allows the monitor 349 adapted for scanning of twelve leads. By snapping of the monitor 349 into the array 180 of positions so allows interconnecting allowing to evaluate 12 leads.

Therefore, one position is sufficient, the other ones to not need to be implemented.

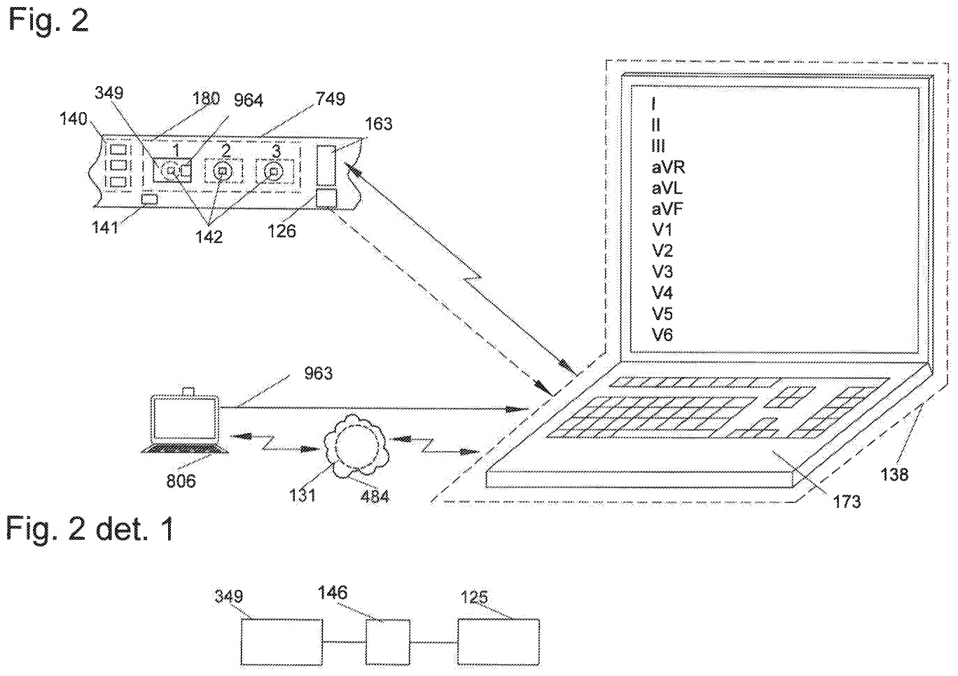

FIG. 2 shows handling during repositioning of the multi-lead monitor 349 in the array of positions to positions for example 1 to 3 or subsequent switching of the respective electrodes by a selector switch 146 from the system 125 of electrodes into the monitor 349 shown in Detail 1, for providing for example of the 12-lead ECG.

For example on the unit 138, in this example formed by a notebook 173, the application “providing of a 12-lead ECG” is set. The display displays the prompt “place the module to the position 1, press START” After recording of the curves for the first position, the prompt “Place the module to the position 2 and press START” is displayed. And so it is proceeded further, till all 12 leads are displayed and transmitted. Preferably, at first, all leads are recorded in the unit 138. Thereafter, communication with the server 806 or the PC 899 is started for their transmitting. Alternatively, the record is used to record subsequently automatically after repositioning of the monitor to positions 1-3, and when the record is complete, it is transmitted to the unit 168 or the server 806. In case of the manual control the push-button 140 of transmitting is pushed for making a record after each repositioning of the monitor 349 to for recording and the push-button 141 for transmitting is pushed after recording of each record to save the record on a memory medium. When the selector switch 146 is used, see, data about the respective lead is transmitted after each switching to another position. Alternatively, the record is saved to an extractable memory medium 964 from which the data is moved by repositioning to the selected unit or server.

Preferably, it is possible to follow the recording quality on the display of the evaluation unit 138 so that the recording can be repeated if necessary.

After transmitting of all leads, on the unit 138 or on the server, the leads are modified into the proper format, either into the usual 6 leads one under the other and the 6 another ones next to it or into another selected format.

Preferably, parameters of the recordings are also displayed, for example the heart rate, the QRS complex, etc., and preferably also the diagnostics, for example whether it is a heart attack case or a AV block of the grade 2.

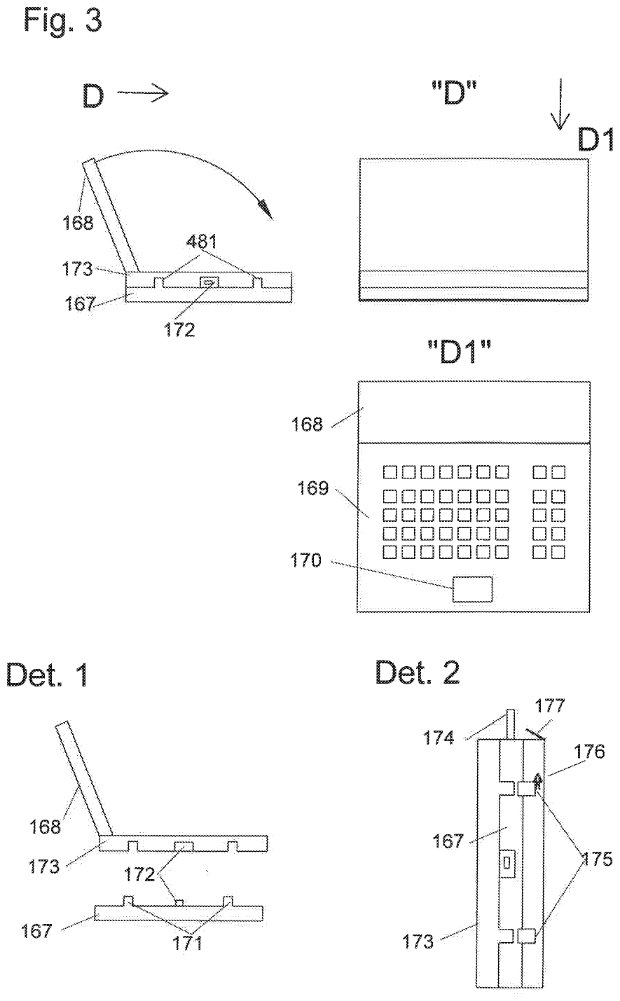

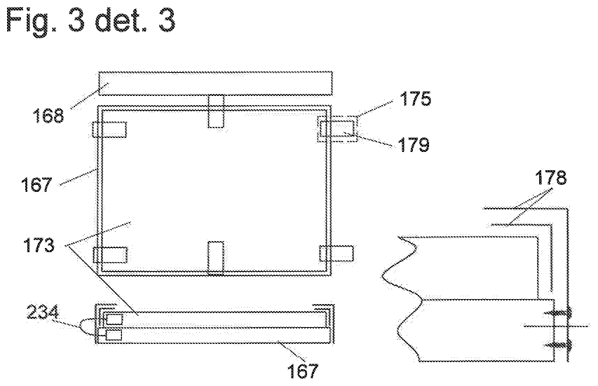

FIG. 3 shows preferably detachable placing of the printer 167 underneath the notebook 173 for the purposes of the ECG for printing of ECG reports directly from the notebook 173. After tilting away of the monitor 168 of the notebook 173 the keyboard is accessible 169, preferably with a mouse 170. This layout is suitable in particular for hospitals, where according to the prior art a non-portable ECG instrument is transported to the patients.

The notebook 173 with printer is easy to transport and allows operative carrying it to the patients.

FIG. 3 Detail 1 shows a disconnected printer 167 attachable to the notebook 173 by means of a mounting mechanism 171 of the printer and a connector 172 of the printer.

FIG. 3 Detail 2 shows a closed notebook 173 with an attached printer 167 and a detachable loop-strap 174 for easy carrying. Preferably on the printer 167 a case 176 is attached by means of a mounting mechanism 175 provided with a storage space accessible by a door for storing of the monitor 349, chest belt 749, the electrodes and of other accessories for the purposes of the ECG scanning.

FIG. 3 Detail 3 shows assembling of a set of a notebook 173 with a printer by means of a mounting mechanism, preferably represented by a universal mounting mechanism 179. Preferably, the universal mounting mechanism 177 comprises a double-angle iron 178 attached to a side of the printer fixing the keyboard part of the notebook 173, over which the monitor is folding to the required position to the printer 167. The double-angle irons are adjustable in a certain extent and allow so fastening of the notebooks of different sizes not exceeding the dimensions of the printer. With the printer they are electrically connected by a cable with connectors, preferably with the USB connectors connecting for data transfer and/or power supply.

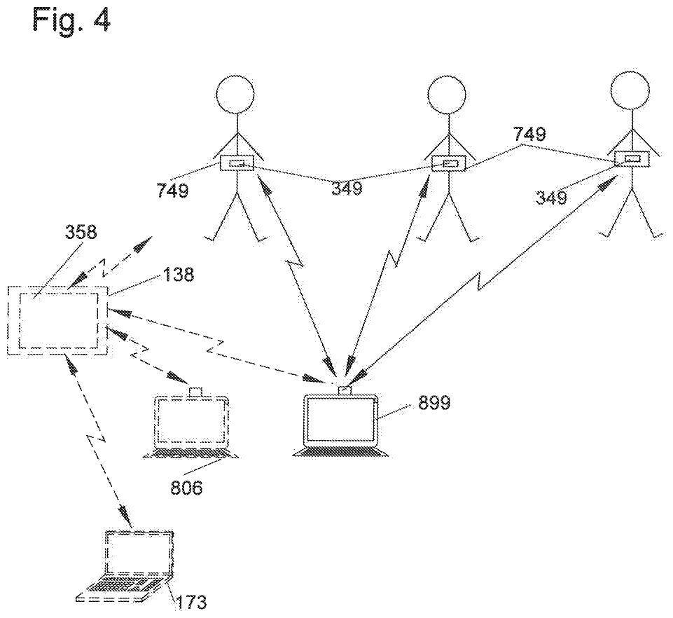

FIG. 4 shows an example of using of the belt 749 with a monitor 349 in the doctor's waiting room, or in an ambulance in the hospital. Patients are monitored at the time of their waiting. Their data are stored, and preferably, an extractable memory medium 964 is used for later transport of data, in particular of the ECG data, to a PC 889 of the physician.

So, the physician will use the patient's waiting for providing of a Holter type recording, which is processed from data stored in a PC 889. Preferably, the observed phenomena are recalculated from the actual recording time to 24 hours, which period of time is the usual for Holter recordings.

Or, the data is transmitted wirelessly already during the waiting time period directly or via a mobile data network, preferably a WiFi one, alternatively, via an evaluation unit 138, for example via WiFi, to a PC 889 of the physician, preferably represented by a notebook 173 or via Internet over a server 806.

The physician can follow the health state of patients already during the waiting period of time and in case the state becomes worse such patient in danger can be treated with preference. Preferably, the states off limits are alerted by an alarm, and preferably, the data and the ECG of the monitored person with data off limits are displayed automatically on the PC 889 of the physician.

On the PC 889, it is possible to select to a display showing more patients and it is possible to switch between them according to needs.

Here as well it is preferable to process the transmissions in the monitor 349 or in an evaluation unit 138 or in a PC 889, or in a server 806 a Holter-like record. The physician can issue instruction to process the record from the monitor 349 also remotely from his surgery room on the PC 889. The record can be produced also by attaching of fingers or palms on the contacts placed on the belt or the monitor instead of placing the monitor on the belt or to the chest.

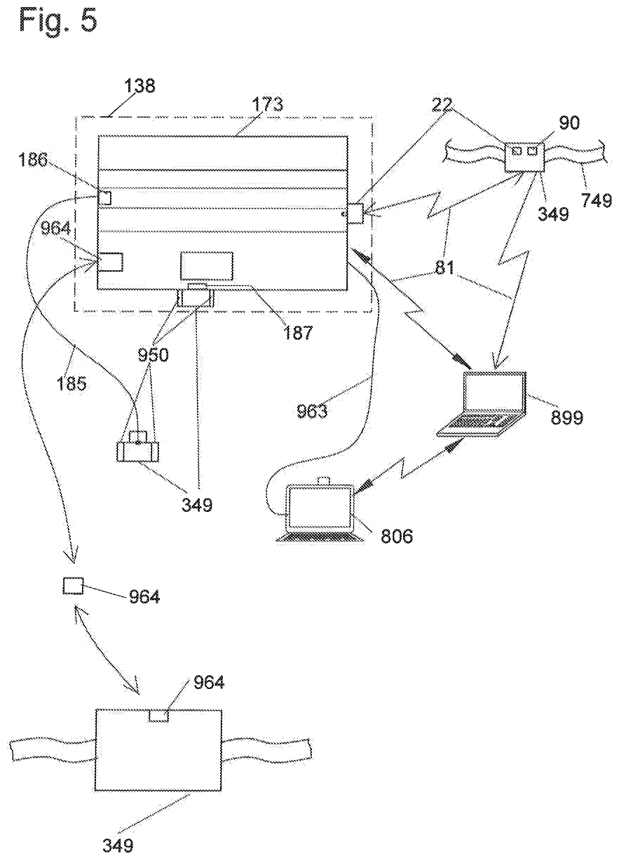

FIG. 5 shows communication between the evaluation unit 138, preferably formed by a notebook 173 and by a monitor 349, which is preferably inserted into a connector 187 for connecting of the monitor to the notebook 173, preferably formed by the USB, preferably placed on the front side of the notebook for easy attaching of fingers to the electrodes 956, so that by connection of the monitor 349 a preferable configuration is formed for scanning of the ECG of the monitored person by means of electrodes 950 and at the same time for observing of a record on a display of a notebook 173 via a connector 187 of the monitor 349, which communicates and is powered so that preferably in the monitor there is not any accumulator or communication wireless module. This makes the monitor 349 smaller and cheaper. Alternatively, the monitor is connected by means of an extension cable 185 into the connector 186 placed on a side. This cable it is possible to use also for connecting to a mobile phone 100. Another alternative is a wireless link, preferably a Bluetooth, for communication from the monitor 349. In comparison to the communication through the connector or cable it required to add an accumulator and a communication module into the monitor 349. The wireless connection allows also connecting to an evaluation unit 138, preferably formed by a PC 889. The notebook 173 and/or the PC 889 are provided with software for data processing of cardiac signals received from the monitor 349 for one-lead to 12-lead ECG, preferably in the ECG format and/or the Holter format, and this will entirely replace the common ECG instruments or Holters. Preferably the reports are printed by printers or they are stored in memory. Transmissions of data of cardiac signals are possible also from the server 806 or from the exchangeable portable memory medium, preferably from a SD card, which data were saved and stored preferably in the monitor 349.

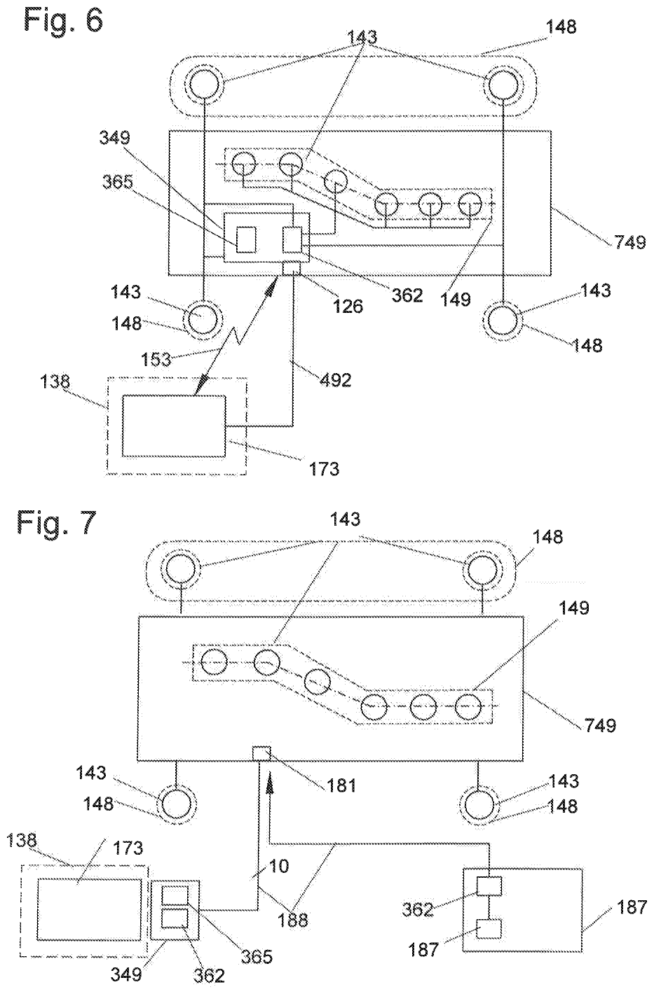

FIG. 6 shows the system of electrodes 149 on the chest belt 749, as well as the system of external electrodes 148 connected by wires into the monitor 349.

In this embodiment the monitor is 349 a part of the belt 749. The monitor 349 comprises a front end 362 and a control unit 365 of the monitor. The monitor with an evaluation unit 138, is formed preferably by a notebook 173 interconnected by wires through the connector 126 of chest belt cable 492 and/or by wireless link 153.

FIG. 7 shows a design of a chest belt 749 without the monitor 349 with the same systems of electrodes 148, 149 as in the example shown in the previous figures, in which the signals from all electrodes 143 are brought together in the belt 749 passed on to the connector 181 of electrodes. Through this connector the signals from the electrodes 143 are brought by means of a multicore cable 188 to the front end 362 circuits in the monitor 349. The controlling unit 365 of the monitor processes them and the result is passed on to the evaluation unit 138, preferably formed by a notebook 173. A monitor 349 is inserted into the connector 186 or 187 of the evaluation unit 138, or it is integrated in it.

Alternatively, the signals are brought by means of a multicore cable 188 to the front end 362 of a desktop ECG 189 and the signals are processed by the control unit 187 for the ECG.

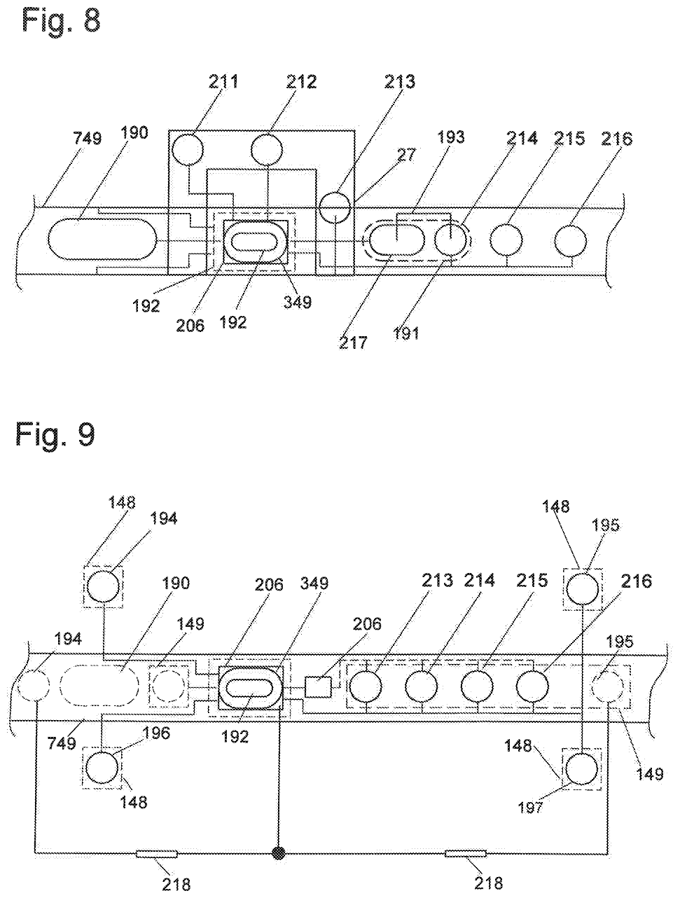

FIG. 8 shows a chest belt 749, which comprises a connector for connecting of the monitor 349, preferably formed by snap fasteners and/or a more-contact-connector. Into the monitor, signals are brought from the left collecting electrode 190 and the right collecting electrode 191. The collecting electrodes are of oval shape and take a larger area than the common ECG electrodes and they so they scan associated cardiac signals that correspond to the signals from several common ECG electrodes. The associated signals comprise not only the signals approximately of the lead I of the ECG, but approximately also those of the leads V3, V4, V5, V6. An advantage is that only by two electrodes placed on the chest belt in the level for measuring of the V4 to V6 it is possible to monitor the associated signal, from which it is possible to display and follow also elevation of the ST segment, which segment is important for evaluation of a heart attack, what is not possible in the common ECG I curves that are scanned from two common ECGs of electrodes. The left collecting electrode 191 is disconnectable by a withdrawable bridge 193 to the larger part 217 of collecting electrode and a part of collecting electrode that forms the electrode 214 V4 for scanning of the cardiac signal at a recommended place for placing of the electrode for scanning of the ECG signal V4, and which is, together with the electrodes 215 V5 and 216 V6, connectible to the monitor 349 for scanning of the signals V4, V5, V6. The so designed chest belt allows scanning from the collecting electrodes 190 and 191 when bridge is connected and when the bridge is disconnected and optionally by the connected electrodes 215, 216 of leads V3 to V6, eventually also the leads V1, V2 by means of a bridge 27 of electrodes against the right collecting electrode 190, which substitutes the Wilson's clip.

FIG. 9 shows the multi-lead chest belt 749, which is adapted for mounting of the monitor 349 for up to 10 leads. The connector 192 of the monitor is adapted for transmitting to the chest belt 749 of signals from the system 148 of external electrodes, and this of electrodes 194 RA, 195 LA, 196 RL, 197 LL, and the system 149 of electrodes on the chest belt, and this of electrodes 213 V3, the electrodes 214 V4, the electrodes 215 V5, the electrodes 216 V6. According to model of the monitor 349 this monitor scans leads V3, V4, V5, V6 simultaneously and/or leads I, V3, V4, V5, V6 are switched sequentially by means of a selector switch 206 of the V-leads.

Alternatively, instead of placing of electrodes 194, 195, 196 off belt the electrodes are placed on the belt. This are the electrodes 194, 195 shown in dashed lines on sides of the monitored person or slightly shifted to the chest, and the electrode 196 shown in dashed lines on the right side of the chest. Optionally, instead of the electrodes 197 LL a clip is used built from the resistors 218 leading from the electrodes 194, 195 that are placed on the belt or it is used the right collecting electrode 190, serving instead of the Wilson's clip. Thereby, all electrodes are placed on the belt 749.

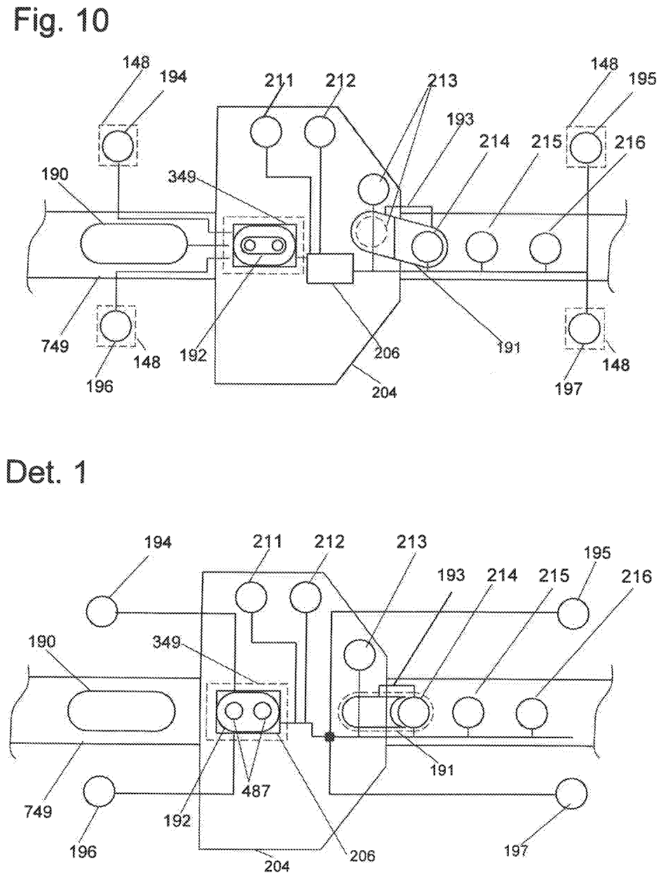

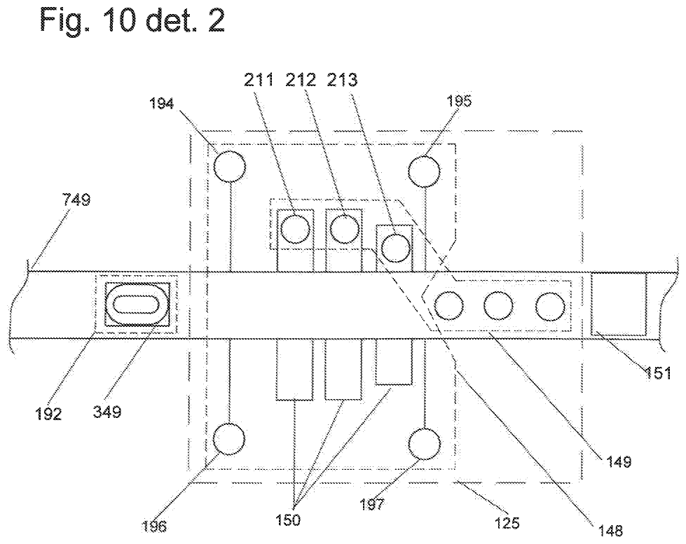

FIG. 10 shows an enlargement of the belt 749 from FIG. 9 by adding a withdrawable additional board 204, which allows scanning also of the lead V1, the lead V2 and the lead V3 in such a way that the electrodes 211, 212, 213 corresponding to them are placed on the board in positions above the chest belt 749 so that they are adjacent to the respective places of the chest of the monitored person for the leads V1 to V3 after attaching of the belt. The required pressure of electrodes to the skin of the chest is achieved so that the plate is widening also to the opposite side of the chest belt 749 and it is pre-tensioned. This design allows, according to the model of monitor 349, simultaneous scanning of up to 12-lead ECG or of a sequential ECG, then the leads V1 to V6 are switched sequentially by means of a selector switch 206 of the V-leads or the monitor is moved to different connectors for connecting to the respective electrodes. This configuration allows to scan without additional board 204 the one-lead ECG from the collecting electrodes 190, 191 with put on withdrawable bridge 193 or the multi-lead ECG with disconnected bridge 193, and this from electrodes 213 to 216 against the collecting electrode 190 and with additional board 204 with a monitor 349, inserted into the connector 192, which preferably substitutes the original monitor, preferably for less leads. After connection of the system of external electrodes 148, it is possible to monitor up to 12-lead ECG. Repositioning of the external electrodes 148 on the belt 749 shown in FIG. 9 is possible.

The Detail 1 shows a design of the chest belt 749 where to the belt is snapped an additional board 204, which board, however, allows direct connecting to it also of the external electrodes, that is the electrode 194 RA, electrode 195 LA, electrode 196 RL, electrode 197 LL. The board is fastened to the belt by means of the connector 192 of the monitor and is pressed by the belt 749 to the chest of the monitored person. Further, the board comprises a monitor 349 comprising circuits for processing of up to 12-lead ECG. This configuration allows a very rapid transition from the informative, one-lead ECG, measuring by using a belt 749 without any additional board 204, preferably with the less effective, one-lead to four-lead monitor by means of the collecting electrodes 190, 161 connected by a bridge 193 and of the multi-lead scanning by disconnecting of the bridge 193 and by using of electrodes 214, 215, 216, measured against the collecting electrode 190. Measuring of the complete twelve-lead ECG is made possible by mere snapping of the additional board and by fastening of the system of external electrodes 198, and this of electrodes 194 RA, 195 LA, 196 RL, and 197 LL already connected to the board 204 preferably with a more effective monitor for 12 leads. Repositioning of the external electrodes 148 to the chest belt 749 according to FIG. 9 is possible.

Detail 2 shows a chest belt 749, which instead of the enlarging plate 204 for scanning of the leads V1 to V3 comprises three independent springs 150 provided on their upper ends by electrodes for scanning of the leads V1, V2, V3. They supplement the electrodes for scanning of the leads V4, V5, V6, that are placed directly on the chest belt 749. These six electrodes form the system 149 of electrodes of the belt. In co-operation with the system of external electrodes 148 for scanning of the lead I between electrodes RA and LA and II between electrodes LA and LL forms together the complete system 125 of electrodes. Therefore, the monitor 349 placed on this belt can monitor eight leads and the four remaining to the complete 12-lead ECG can be calculated.

Preferably, the belt 749 comprises a position for additional accumulator 151 of the belt for extension of the operation time of the monitor 349. Thereby, functionality of its accumulator is prolonged and/or backup.

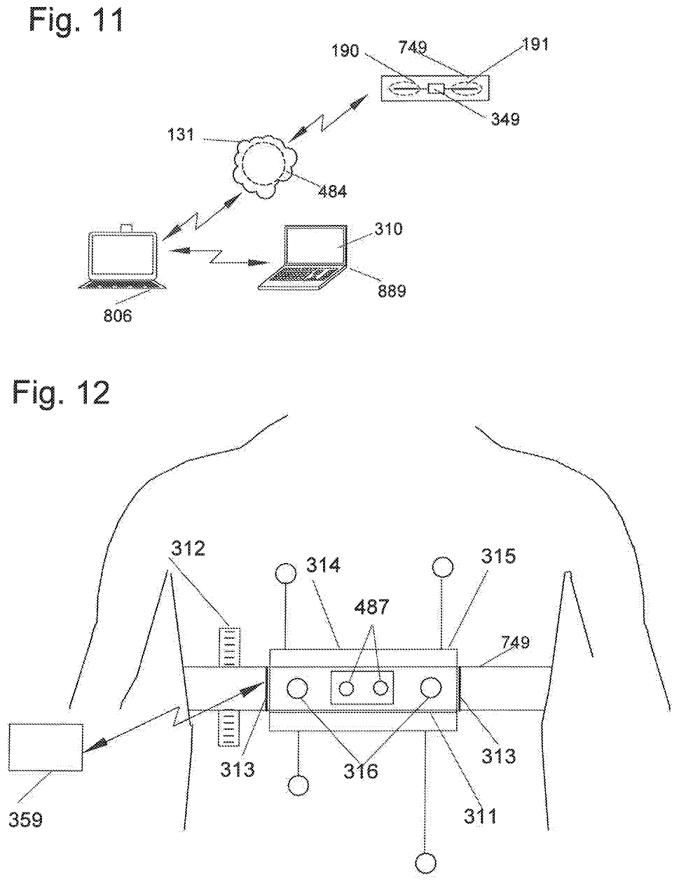

FIG. 11 shows chest belt 749 preferably with collecting electrodes, and this with the right collecting electrode 190, and the left collecting electrode 191 and a monitor 349 of cardiac, placed on the chest of the monitored person 2, for scanning of the 1-lead ECG. Alternatively, may be used a universal chest belt 749 with electrodes and a monitor for scanning of the multi-lead ECG, where it is possible to use selectively either the collecting electrodes 190 or the electrodes for the multi-lead ECG. Optionally, the belt is attachable to the chest and held there or it is provided with stretchy straps 198 for attachment to the chest by means of mountings 199. The collecting electrodes are placed in places, where the ECG electrodes are not commonly placed and they scan so that it does not correspond exactly to any lead of a common ECG, but approximately they are similar to the lead I and partially cover the leads V3 to V5. An advantage is that they show collectively the S-T section, partially from more leads.

FIG. 12 shows the chest belt 749 shown in FIG. 11, which is used for fastening of the attachable monitor 349 for scanning for example of 8-lead to 12-lead ECG. Pro fastening, preferably, monitor 349 is taken from the e belt 749, which is fastened by snap fasteners 487 and the chest belt 749 is moved from the position below nipples, which serves for scanning of the one-lead ECG, to the position for scanning of the multi-lead ECG so that its chest electrodes 316 are in the correct place, preferably between the 4th and the 5th rib, centrally against chest bone. Position of the monitor 349, preferably formed by a sensor of the multi-lead ECG, preferably of the 8-leads to 12-leads, is secured by the guide rail 311 of the monitor 349, which is attached to the chest belt by placing of the sensor. After correct placing of the monitor 349 on the chest so that electrodes are in correct place so as it was mentioned above, position of the chest belt is measured in relation to the nipples, preferably by means of the foldable scale 312 laced on the chest belt, which scale is unfolded to the measuring position. Preferably, this position is marked on the foldable scale 312 for the next easier placing of the attachable chest belt 349. By activation of the respective push-button on the monitor 349 scanning of ECG is activated, which is by means of the system of three electrodes via wires connected to the monitor 349, for the left and the right arms and the right leg, what is preferably supplemented also by a reference electrode for the right leg. Up to eight-lead ECG is recorded. The Record of the ECG, stored in memory is preferably after activation of the push-button for transmission, transmitted into the evaluating unit 138.

In case of making a record also for another leads of the ECG for the chest leads V3 to V6, the monitor 349 is moved to the position for measuring of the leads V3, V4.

A record in the evaluation unit is carried out after activation of the push-button for record for recording of up to eight-lead ECG, including the chest leads V1 and V2. In case of repositioning for the chest leads V3, V4, by repeated pushing of the push-button transmission the record for leads V3, V4 is also recorded in the memory. Similarly for another repositioning of the monitor 349 for recording of the leads V5, V6 after their recording, the push-button for transmission is pushed again, which is recorded in the memory. Thereafter, it is possible to use from the memory of the evaluation unit 138 the record for displaying it in this unit. The records from all leads will be there filed one under the other, so as it is usual in a ECG record. For transmission to server activation is carried out in the evaluation unit 138 of the respective control equipment. Preferably, the evaluation unit 138 is formed by a mobile phone or a tablet or a notebook or a desktop computer or a displaying unit. In case that a tablet is used by the physician, it is not necessary to make a record, because the record displayed on this tablet can be carried with it and it can be presented to the health care staff so as it is necessary. From the tablet, as well as from other units that form the evaluation unit, it is possible to make a record by transmission in a printer or directly from a unit provided with a printer placed for example as a desktop instrument for ECG, to which it is possible to transmit the cardiac signals. When used in the hospital environment, preferably the one-lead ECG is scanned by means of the chest belt shown in FIG. 11, where it is possible by transmission into the control room, preferably via the local WiFi and a server, to display in a PC screen divided into more partial images records for more patients. In case of an automatic alarm when the ECG values for a patient scanned by the multifunction chest belt exceed the preset limits, the screen shows automatically data of this patient. In this case preferably the possibility is used to scan a multi-lead ECG by the monitor 349 preferably formed by the attachable sensor XX and so a 8-lead to 12-lead ECG is obtained immediately, by mere touching of one sensor and preferably it is fastened by the chest belt 749. It is much more rapid and simpler attitude than when a desktop ECG apparatus on a trolley is brought to the patient so as it is now the common practice. An advantage is that the chest belt is already put on, and therefore, it is not necessary to carry out another handling and holding of the sensor by this chest belt is easier than holding of the sensor by the medical staff. Moreover, it allows a more rapid and more accurate putting on of the sensor to the desired place. Also, an advantage is providing of a ECG record in the evaluation unit formed by a notebook, because in particular in the hospital environment, it is easier transportable than a desktop ECG on a trolley. Moreover, there is no need to make a printed copy, because a notebook can be carried away by a medical staff member and it can be shown to the physician to see and review the record and a record can be printed on a common printer in case is necessary. From the notebook, as well as from the tablet it is possible without printing immediately after seeing it by the physician to store the record in memory on a hospital server with a relation to the respective patient for next seeing it.

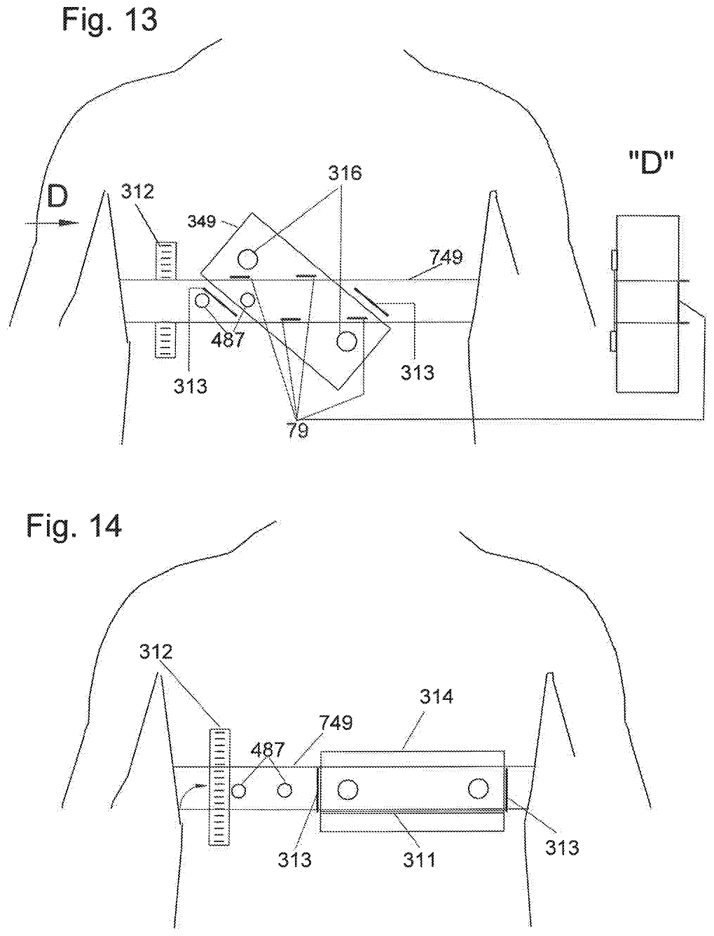

FIG. 13 shows a monitor 349, which is attached to the chest of the monitored person 2 and keeping of the correct angle of placing of the monitor 349 with regard to the belt 749 is ensured by bridges 79 placed on the monitor 349, through which passes the belt and which are formed preferably by a U profile for securing of the belt position. Instead of using a bridge, the correct position on the monitor 349 can be drawn, to reach the correct angle to the belt. For this placing preferably the belt is placed under the position, shown in FIG. 12, in which the chest leads V1 and V2 were measured. For next reference for quick placing of the belt and of the monitor 349 into the correct positions used the foldable scale 312 for finding of shift pf the chest belt 749 from the originally measured position, i.e. with regard to the nipples, which can be marked on the a scale for future use.

FIG. 14 shows attaching of the monitor 349 for measuring of the chest leads 5-6, this time again in the horizontal position in parallel to the chest belt 749. This belt is fixed to the monitor 349 by means of a guiding rail 311. After attaching to this guide rail 311, the chest belt is shifted to the respective position, which position is preferably measured by means of the foldable scale with regard to the nipples for future reference. The snap fasteners in the middle of the belt preferably serve for correct centering of the belt with regard to the chest axis and for correct placing of the monitor with regard to the belt, and so with regard to the chest it is positioned by means of the marking on the chest belt 749.

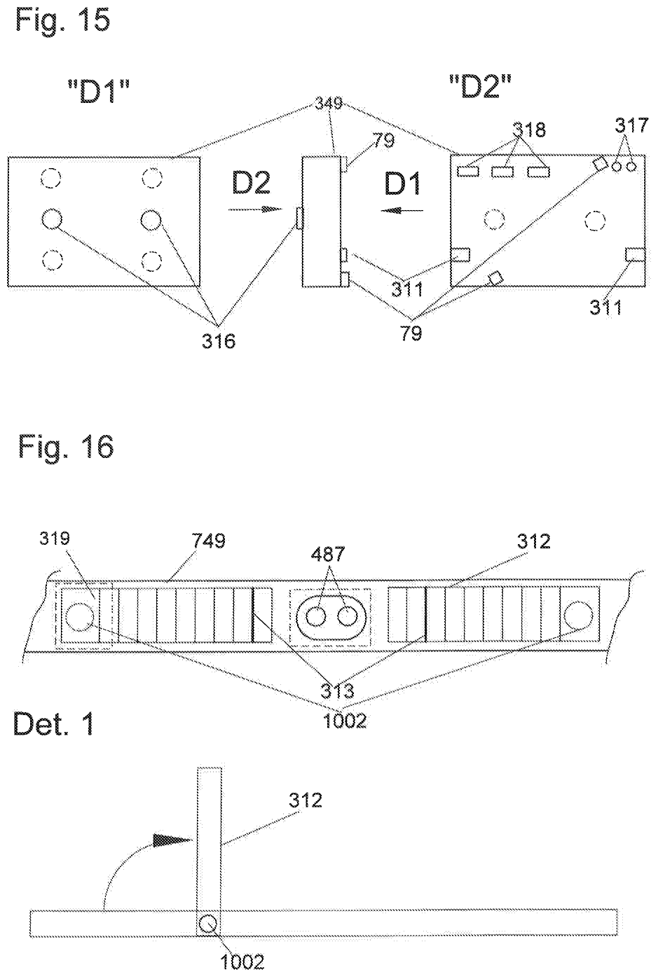

FIG. 15 shows a monitor 349 preferably formed by a sensor 315 attachable for scanning of the ECG signal by means of the electrodes placed on it from the bottom that for attaching of the sensor to the chest have contact with skin. The electrodes are made of some conductive material, preferably a metallic one, and they are slightly convex to ensure contact, as it shows the view in the direction “D1”. Preferably, these two electrodes can be formed each by two electrodes that are interconnected for better signal intensity. In this case the center between the electrodes is taken as the resulting electrode for definition of the place of electrodes on the body. View D2 shows the monitor 349 in view from the side with the guiding rail 311, which preferably is interrupted, and bridges 327 for oblique guiding of the belt. On the front panel also the control push-buttons 318 and the indication LED 317 are placed.

FIG. 16 shows a chest belt with foldable scales 312 around hinge 1002. The basic position of the chest belt is ensured, e.g. by the Velcro hook-and-loop fastener. Preferably, marking of the placing of the monitor 349 is carried out on the belt 749 to make easier the repeated measuring, so that it is not necessary to read from the scale again. Alternatively, the attachable monitor 349 formed by a sensor 315 is attached to the chest and it is held by hand without use of the chest belt 749. An advantage of using the belt 749 for fastening of the monitor 349 on the chest is the possibility to measure its exact placing by means of a foldable scale 312 or marking 313 on the belt, further of the bridge on the monitor 349 and the centering of the belt by means of snap fasteners. Another advantage is the case of the long-term measuring by means of the belt 749 with the collecting electrodes with a monitor 349 for one-lead ECG, when this belt is already attached to the chest which it encompasses, and therefore it is used for holding of the monitor 349 on the chest it is not necessary to place this belt 749 on the chest, but it can be directly used to press the monitor 349 during its appropriate shifting on the chest. Thereby, the handling is simplified in comparison to the case, when the chest belt has to be installed.

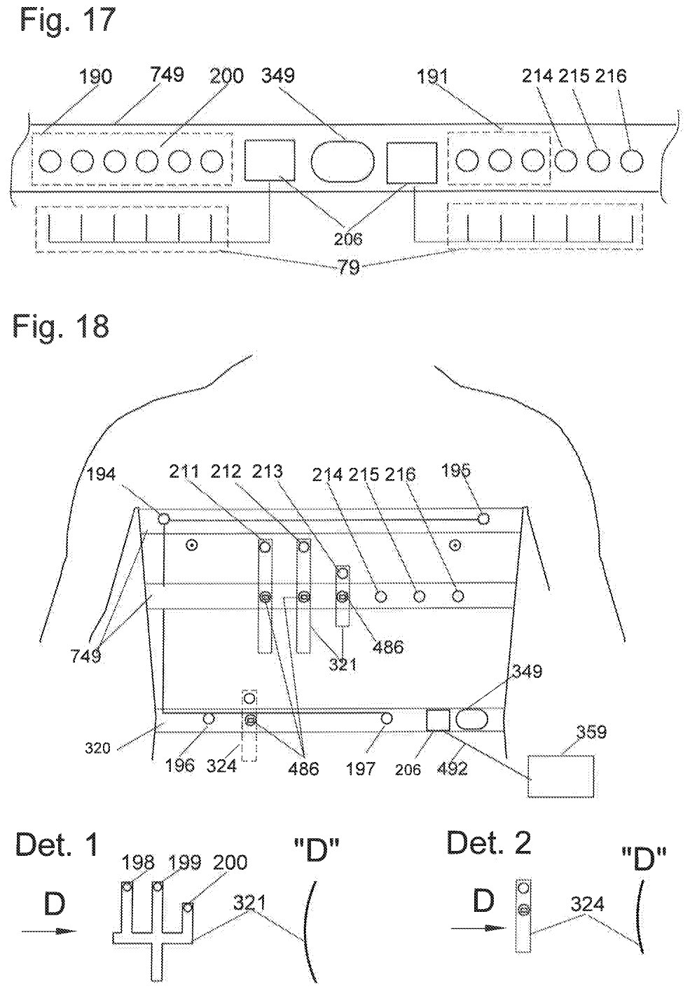

FIG. 17 shows a chest belt 749 with collecting electrodes that consist of electrodes 190, 191 that are optionally connectible together by bridges. So it is possible to choose from the electrodes one collecting electrode of various size indifferent positions on each side of the chest belt, which consists of certain number of electrodes.

In case the collecting electrode on the right side of the belt comprises all electrodes that it is not necessary to switch over, preferably it is formed without any switching over only as one longer electrode. Preferably, on the left side of the belt the electrodes are switched over to make association of electrodes into a bigger one possible or it is possible to let them separated for measuring from the electrodes V3, V4, V5, V6 independently, and not as a collecting electrode. The electrodes are switched over by a bridge 79, which bridge interconnects only the selected electrodes, or they are switched over by the selector switch 206 of the V-leads. V this belt electrodes connected to it can be selected individually as needed. Advantage of this arrangement is that all electrodes are placed on one narrow belt in case of measuring in comparison to the collecting electrode on the right side of the belt, which can be assembled selectively from any number of electrodes placed there, whereby the Kranz's clip 200 is obtained, which is used in this case instead of the classical Wilson's clip for measuring from the electrodes on the other side of the belt, V4, V5, V6 that are selected subsequently by a selector switch, or at once depending on to how many inputs the monitor 349 is adapted. The advantage consists in placing them on one narrow belt in comparison to the classical Wilson's clip, where it would be necessary to place apart from this belt another 4 electrodes. Thereby measuring of the modified chest signals V3, V4, V5, V6 is obtained. In case of measuring from the electrodes 190, 191 against each other the associated ECG signal is obtained approximately corresponding to the lead I and V3 to V6.

FIG. 18 shows a chest belt 749 already shown in FIG. 17, from which will now be used preferably only the electrodes for the chest leads V4, V5, V6 in such a way that the bridge is removed and the electrodes are connected over the selector switch into the monitor 349. The electrodes are measured against the classical Wilson's clip consisting of electrodes for the left arm LA and the right arm RA, placed on the chest belt 749 under the armpit connected by wires together with the electrodes V4, V5, V6 to the selector switch, and further to monitor 349 that are placed on the side belt to the waist, on which are placed the remaining two electrodes of the Wilson's clip for the left leg LL and the right leg RL, also connected to the selector switch by wires. If it is necessary to measure also V1, V2, V3 the segment of electrodes 321, shown in Detail 1, is connected by the connecting element 328 to the chest belt 749, placed under nipples, where the connecting element 328 fastens and simultaneously connects electrically the electrodes V1, V2, V3, placed on it, to the system of electrodes. The segment is made of a stretchy material, preferably of steel sheet, it is slightly convex, as it is shown in the view D, so that when it is pressed by the chest belt 749 it straightens and so presses the electrodes V1, V2, V3 to the skin for good contact. The selector switch is used in the case that monitor is used, which is adapted for measuring of three leads, i.e. the 1, 2 and V1, wherein the three leads AVL, AVR AVF are calculated, that is from totally seven leads, by connecting of four electrodes, the Wilson's clips and the V1 or V4. When measuring with the remaining five chest leads, the monitor is switched subsequently by the selector switch for subsequent measuring and recording in the evaluating unit 138. In case of using the monitor, which is adapted for simultaneous measuring 12 leads, by connecting of 10 of electrodes, the selector switch is not necessary. In case when the RL electrode for the right leg is placed off the side y belt 320, the strap 324 of electrodes is used, which is shown in Detail 2, which is again of stretchy material and slightly bulged again. The system of electrodes placed on 3 belts, as it is shown in FIG. 18 is preferable in that all belts are narrow and easy to put on and it is not necessary to use any glued electrodes. All electrodes are already interconnected by wires in this system, so that no subsequent connecting is necessary. To separate the belts for their storing, the wired connections are placed preferably on connectors, and they are separable. Preferably, the segment 321 of electrodes and the strap 324 of the electrode are connected to the belts by a connecting element preferably formed by snap fasteners that not only connect the segment or strap mechanically but also connect it electrically to the system of electrodes.

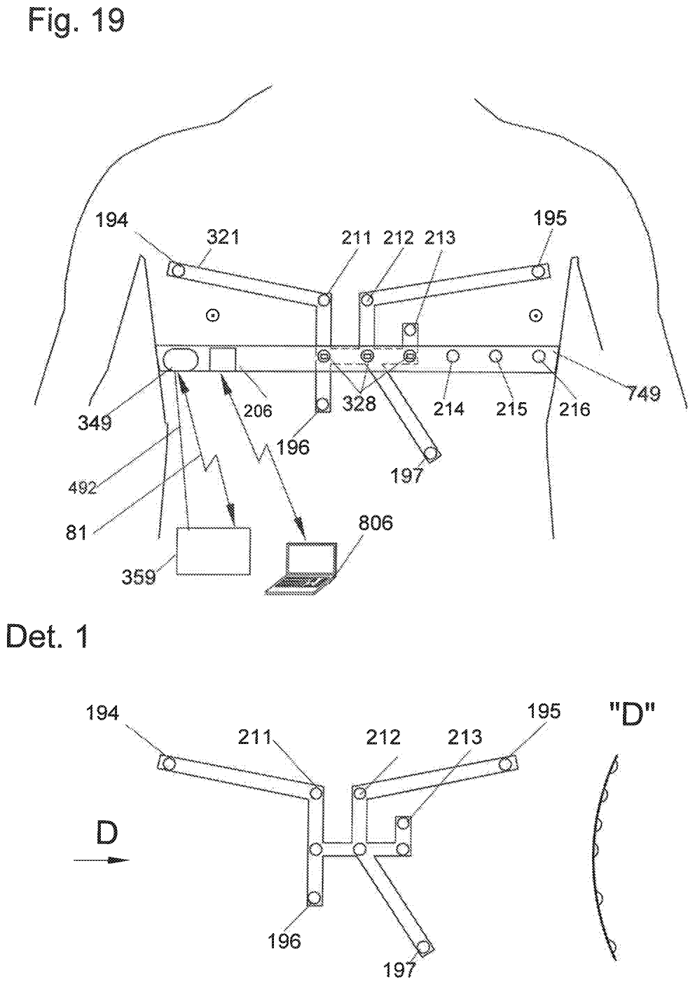

FIG. 19 shows the chest belt 749 already shown in FIG. 17, of which preferably electrodes 214 V4, 215 V5 and 216 V6 are used for measuring of leads V4, V5, V6, that are subsequently switched by the selector switch 206 of the V-leads to the monitor 349. To this monitor this selector switch switches also the electrodes allowing to apply potential from the classic Wilson's clip LA, RA, LL, RL, which are placed on the connecting segment of electrodes 321, which is connected to the chest belt 349 by a connecting element, preferably formed by snap fasteners. This connecting element 328 is shown in Detail 1, where it is apparent that the electrode for V3 is also placed on it. Preferably, this element is made of stretchy material, it is slightly bulged so that it is flattened when it is pressed by the chest belt, whereby a force is produced acting on the electrodes so that they sit well on the skin. In case the monitor is used, which is adapted for measuring by ten electrodes, that is for measuring of all twelve leads at once, it is not necessary to switch between the electrodes, and the measuring will be carried out at one time. From the monitor the measured data are transmitted into the evaluating unit 138 by wires, or wirelessly or the data are transmitted directly into the server, where they would be otherwise transmitted by the evaluating unit if required. This arrangement, shown in FIG. 19, is advantageous in that it is possible to use the narrow chest belt 749 for measuring of one lead or alternatively also of the leads V4, V5, V6 by the Kranz's clip, and in case of the classical measuring by the Wilson's clip the same chest belt is used preferably by connecting of the segment 321 of electrodes and by disconnecting of the bridge for selecting of electrodes. So it is achieved that the complete system of electrodes is already mutually interconnected and it is not necessary any other connecting of the individual electrodes, what is an advantage for easy installing. Also, it is an advantage that it is not necessary to use the glued electrodes that are uncomfortable.

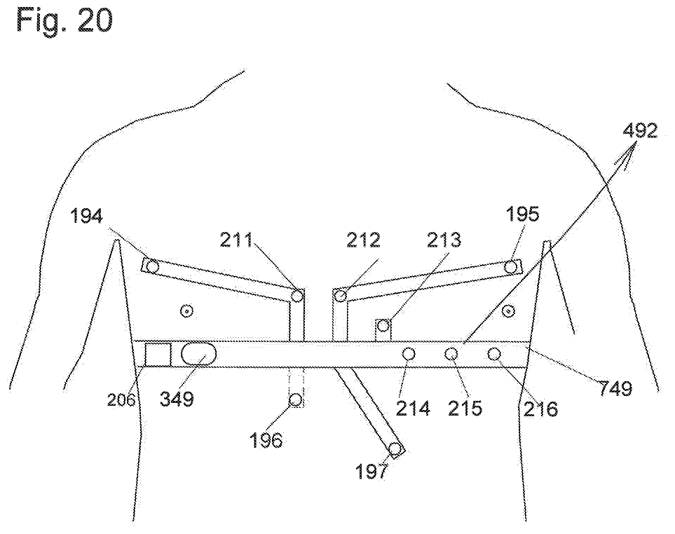

FIG. 20 shows the same system of electrodes as in FIG. 19 but the segment of electrodes is diminished for easier storing installation and carrying, for the price that resulting measurement may not be so accurate, as it is in case of placing the electrodes in their classical positions shown in FIG. 19.

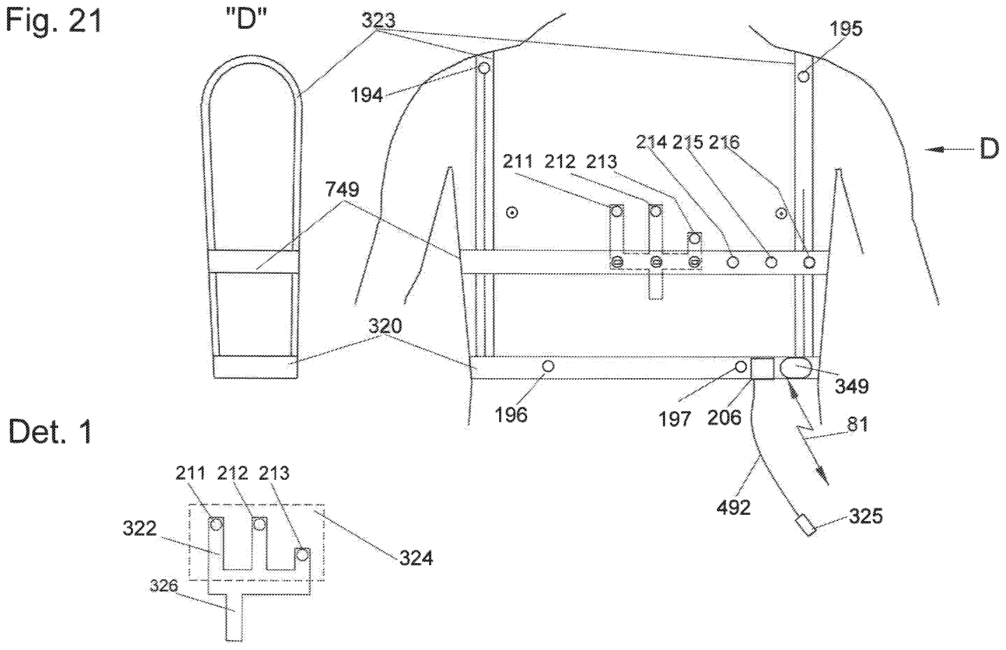

FIG. 21 shows the system of electrodes, which is similar as the system of electrodes, shown in FIG. 18, wherein the electrodes 194, 195 are placed on braces for electrodes for the right arm RA and the left arm LA, by which they are interconnected to the remaining electrodes of the system of electrodes. Said braces are fastened to the remaining two belts, to make possible to connect by them preferably the electrodes RA, LA to the rest of the system of electrodes. Thereafter, the braces are put over the shoulders and they return back to the chest belt 749. An advantage of this system is that the belts and the braces are interconnected into one set and completely mutually also electrically connected, so that there is no need to interconnect individual electrodes and at the same time the braces hold belts in the selected position wherefore they are adjustable in length. If it is desirable to measure the leads V1, V2, V3 the segment of electrodes is used and for placing of the RL off the side belt the strap of electrodes is used.

FIG. 22 shows places on the chest, where the electrodes for scanning of ECG signals, indicated as V1 to V6, are placed.

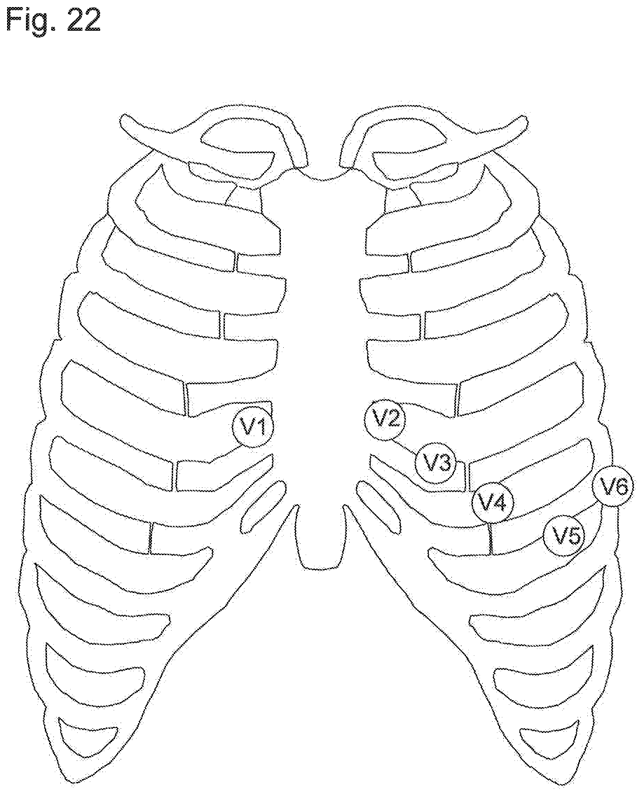

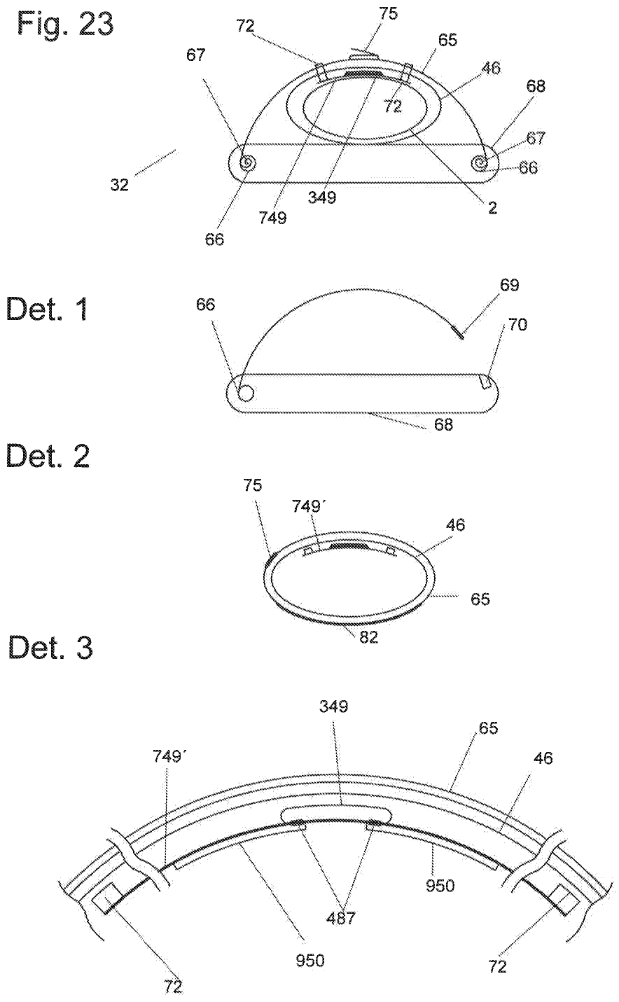

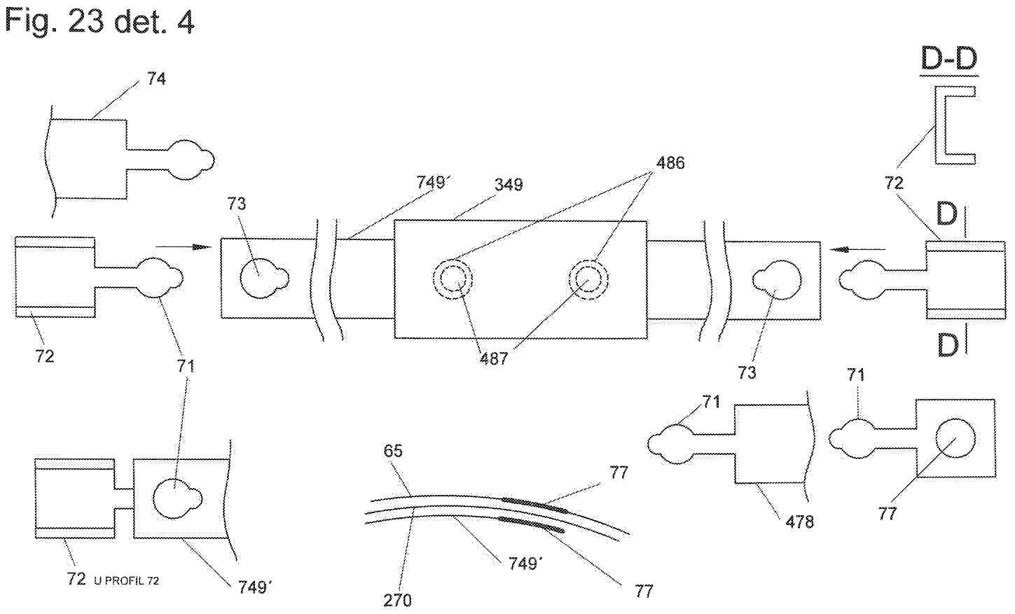

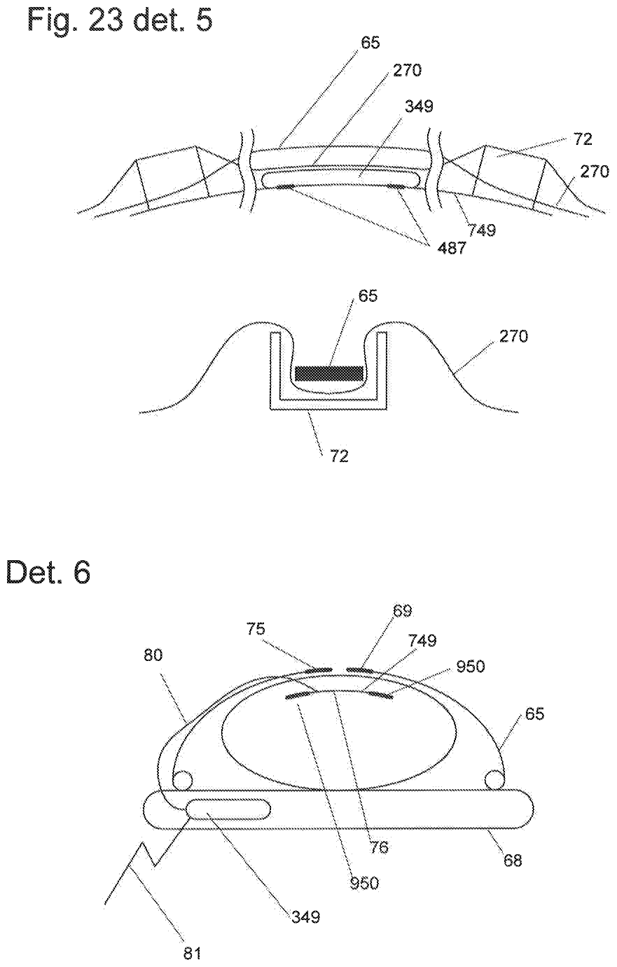

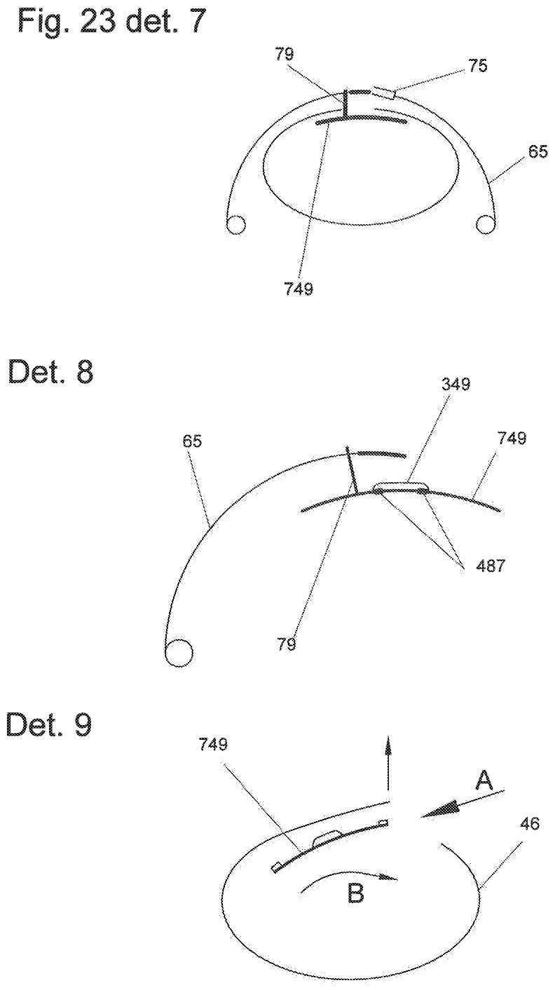

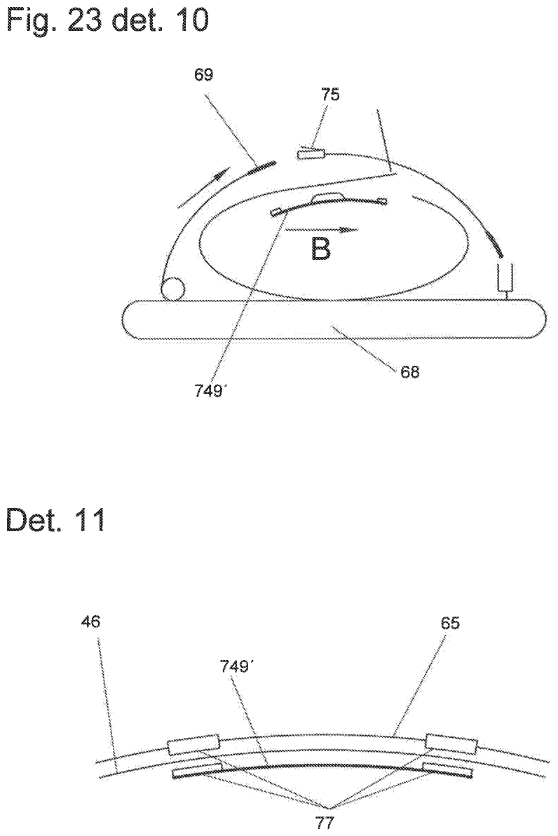

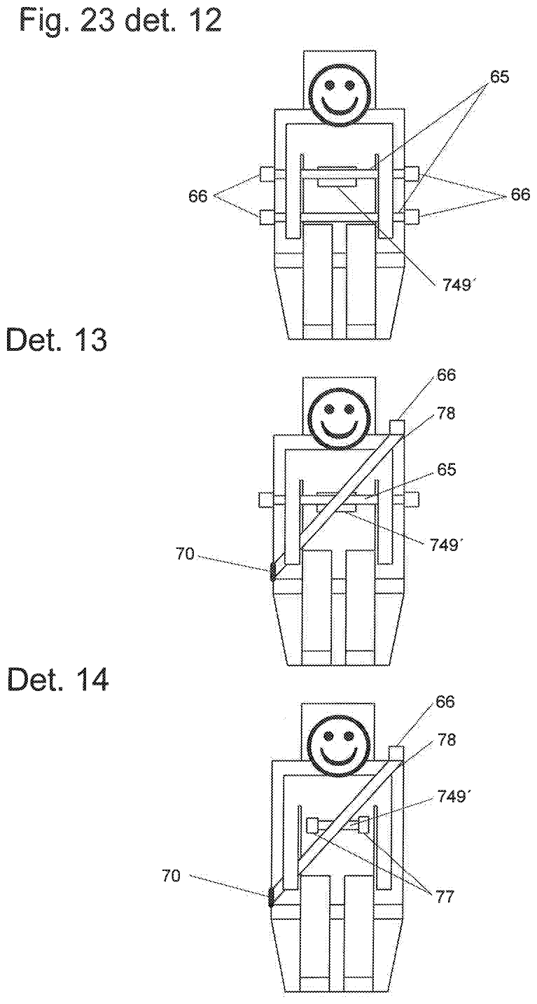

FIG. 23 shows a chest belt 749′ with a monitor 349 under the clothing 46 preferably represented by a shirt 270 or a T-shirt 905 pressed to the chest by the pressure exerting belt 65 preferably comprised of two parts in the middle connected by a buckle 75, that are wound on winches 66 provided with springs 67, which, after being pulled out, re-wind the parts of the pressure exerting belt 65 when they are released again back into the winches 66. Preferably, the pressure exerting belt 65 is comprised of two parts, wherein each of them is wound on one winch, which winches are placed on both sides of the backrest 68 of the vehicle seat 33, which belt is connected by a buckle 75 preferably placed in the middle between the winches. This arrangement is adapted to allow free movement of the monitored person 2 controlling the means 32, what may be a transportation means, a machine and/or a mechanism. When moving forward or sideways it is correspondingly unwound the pressure exerting belt 65 from the winch 66 or it is wound back into it. The springs 67 provide for tensioning of the pressure exerting belt for pressing of the chest belt 749′ for scanning of cardiac signals, or the belt is pulled out of one winch and pulled in on the other side of the backrest by a latch 69 into the opening of the mounting 70 and provided with a releasable anti-burst protection as it is shown in Detail 1.

FIG. 23 Detail 2 shows the pressure exerting belt 65, which surrounds the chest, and is wearable also outside the vehicle. Pressing of the chest belt 749′ to the chest of the monitored person is provided by its flexible part 82.

FIG. 23 Detail 3 shows a chest belt 749′ inserted under the clothing 46, preferably represented by a shirt 270 with electrodes 950 and a monitor 349, preferably withdrawably fastened by snap fasteners 487 with pulled on extensions 71 with U-shaped profiles 72 that hold the pressure exerting belt 65 pressing the chest belt to the chest in the position above it by snapping of the pressure exerting belt 65 across the clothing 46 into the U-shaped profiles 72.

FIG. 23 Detail 4 shows the chest belt 749′ with a monitor 349 preferably fastened by snap fasteners 487, with openings 73 for mating extensions 71 with U-shaped profiles 72 or magnets that are preferably placed there instead of the elastic strap 478 used for fastening on the chest. The pressure exerting belt 65 is snapped by means of magnets 77 to the chest belt 749′ by the action of magnetic force across clothing, whereby an assembly is formed, and therefore the chest belt 749′ is fixed in the position defined by the pressure exerting belt 65. Similarly, when the pressure exerting belt passes through the U-shaped profile 72, it presses the chest belt 749′ to the chest. This figure shows the segments with the U-shaped profile 72 that are fixed to the chest belt fixing them by building them in the openings 73, by which the common chest belt is provided for snapping of the stretchy belt guided around the back of the monitored person. Alternatively, the U-shaped profile is placed in close proximity of the monitor and/or the case of the monitor is provided along the whole side turned to the pressure exerting belt 65 by a groove into which the pressure exerting belt snaps.

FIG. 23 Detail 5 shows the pressure exerting belt 65, which snapped into the extension 71 with U-shaped profiles.

FIG. 23 Detail 6 shows the halved pressure exerting belt 65 that is pulled out of the winches 66, preferably placed in the backrest 68, the halves of which are connectable by means of a buckle 75 and a latch 69 insertable into the buckle 75 with a releaseable lock against ejecting out. To the pressure exerting belt 65 is preferably fastened the chest belt 749′, preferably by means of fastening of the belt 749′, preferably formed by the bridge 79. Alternatively, link 80 passes through the pressure exerting belt 65 to the monitor 349 placed preferably in the backrest 68, wherefrom it passes on the signal via a wireless link 153 or a wired link 492.

FIG. 23 Detail 7 shows the chest belt 749′, which is adapted for fastening it to the pressure exerting belt 65 by means of a bridge 79, which bridge is insertable from the outside after opening of the clothing 46, preferably represented by a shirt, whereby its pressing the chest belt 749′ to the chest of the monitored person is provided. The pressure exerting belt 65 may be fastened by a buckle 75.

FIG. 23 Detail 8 shows, for the sake of clarity without displaying of the clothing 46, one side of the pressure exerting belt 65 provided with a fastening of the chest belt 749′ by the bridge 79 in one place.

FIG. 23 Detail 9 shows inserting of the chest belt 749′ through the unbuttoned clothing 46, preferably represented by a shirt, at first in the direction of the arrow A.

FIG. 23 Detail 10 shows shifting of the already under the clothing inserted chest belt 749′ in the direction of the arrow B towards the center of the chest, with subsequent buttoning of the clothing 46, wherein the chest belt is now prepared for securing it in the proper position by the pressure exerting belt 65, which acts by force in the direction towards the backrest 68 and it is fixed on the other side of the backrest 68 by the latch 69 inserted into the buckle 75.

FIG. 23 Detail 11 shows the chest belt 749 and the pressure exerting belt 65 provided for fixation with the connection of the chest belt 749 and the pressure exerting belt 65 instead of the U-shaped profile by magnets 77 that are fastened both to the chest belt 749 and to the pressure exerting belt 65 so that they act on each other through the clothing 46 and keep the chest belt in the required position on the chest.

FIG. 23 Detail 12 shows implementation of the safety belts, instead of the usual method in the form of a sash obliquely put over the chest by means of two pressure exerting belts 65 in the waist level and in the chest level for increased safety during the simultaneous pressing of the chest belt 749 to the chest by one of the pressure belts 65, preferably the upper one, which serves simultaneously as the safety seat belt, while the second belt 65, preferably the lower one, serves preferably only for safety.

FIG. 23 Detail 13 shows a combination of the safety belt 78 placed obliquely over the chest with the pressure exerting belt 65 placed horizontally over the chest, for pressing of the chest belt, which preferably serves as a safety belt to increase the level of safety.

FIG. 23 Detail 14 shows the safety seat belt 78 placed obliquely over the chest, which serves simultaneously for pressing of the chest belt 749 inserted under the clothing 46 secured in the position preferably by magnets 77 on the chest belt with counterparts preferably on the external side of the clothing 46.

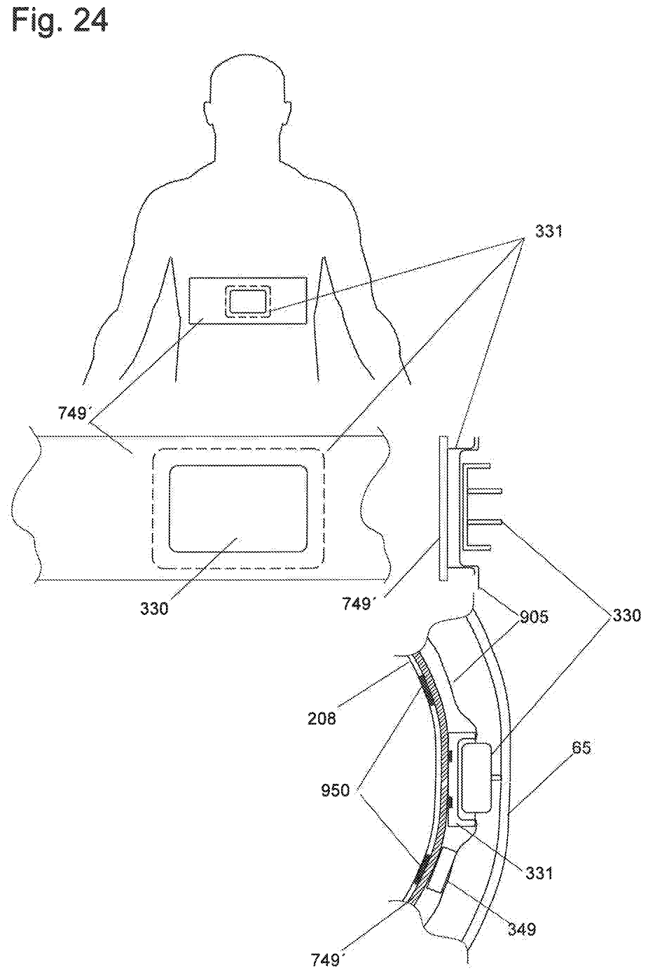

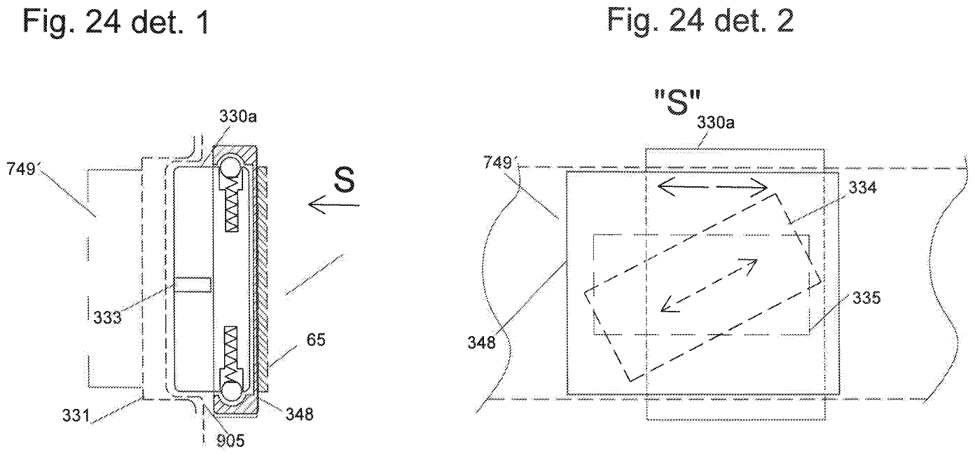

FIG. 24 shows using of the fixation block 331 for pressing of the shortened chest belt 749′ to the body 208. The fixation block 331 is connected with the chest belt 749′ permanently. After inserting of the belt under the T-shirt 905 or other piece of clothing, which cannot be unbuttoned on the front side, into the recession formed by a fixation block 331 through the clothing is inserted a pressure element onto which acts the pressure exerting belt by a force in the direction towards the chest of the monitored person 2, and thereby it presses the electrodes 950 to the skin. Because the fixation block 331 is placed in the middle of the shortened chest belt 749′, the monitor is 349 placed off the middle.

FIG. 24 Detail 1 shows a fastening pressure element separable from the pressure exerting belt 65. The pressure exerting belt 65 is connected with the adaptor for fastening the adaptable pressure element 330a. The adaptable pressure element can be used both in the use of the fixation block 331 and in the use of the bridge 79 with a spike 332.

FIG. 24 Detail 2 shows in view “S” the pressure exerting adaptor for the pressure exerting belt 65 which is horizontal and oblique.

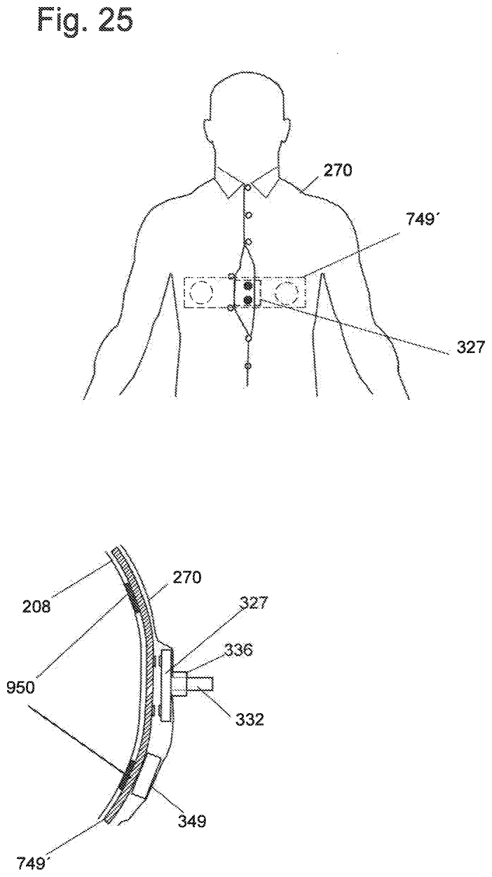

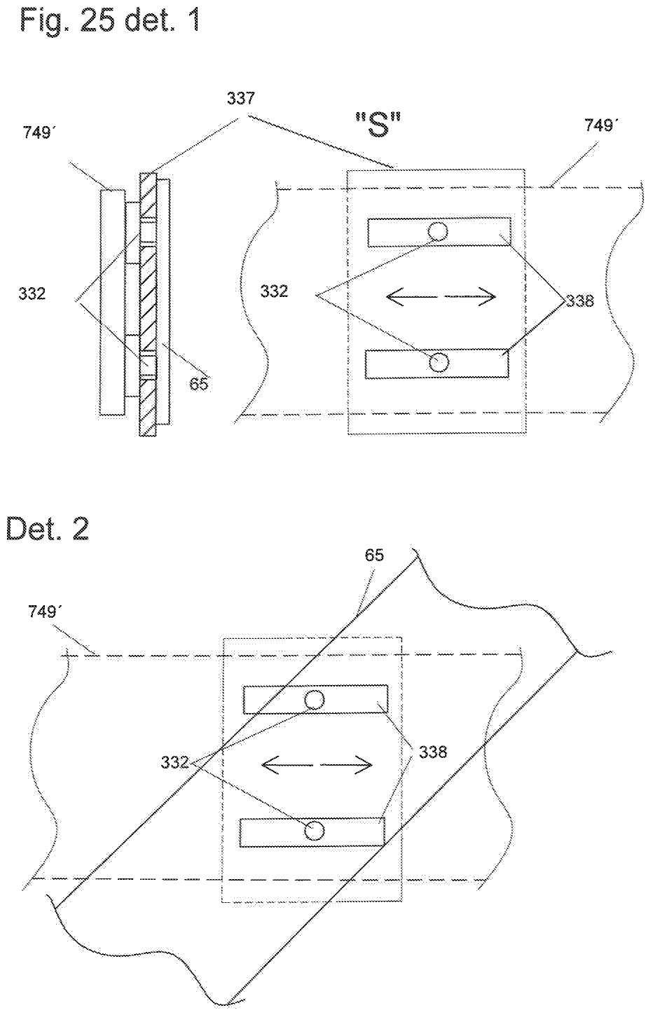

FIG. 25 shows placing of the shortened chest belt 749′ under the shirt 270 and passage of the spike 332 of the bridge 327 through an opening in the shirt 270.

FIG. 25 Detail 1 shows using of the template 337 for making turning of the monitored person 2 on a seat of a means possible when the horizontal pressure exerting belt 65 is used. The shortened chest belt 749′ is provided with spikes 332 with a shoulder. The spikes fit into cutouts 338 in the template. The template 337 is exposed to action of pressure exerted by force of the pressure exerting belt 65, which force is passed on to spikes and through spikes it acts on the chest belt 749′.

FIG. 25 Detail 2 shows using of the template 337 for turning of the monitored person 2 on a seat of a means when the oblique pressure exerting belt 65 is used.

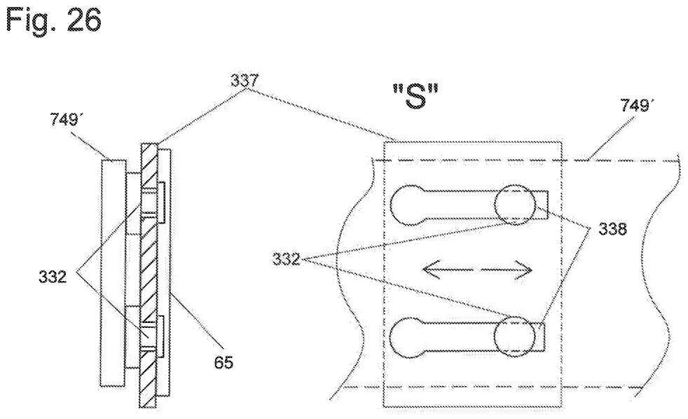

FIG. 26 shows using of the template 337 and the spikes with a groove. Preferably, the spikes are provided with grooves for receiving the cutouts 338. In some place, preferably at the end of the cutouts 338, the cutouts are enlarged so that spikes 332 with grooves can be pushed through the template. After the following movement in the direction to the middle of the template 337 a connection of the template 337, the chest belt 749′ and the pressure exerting belt 65 is formed. Thereby, the chest belt 749′ with the spikes 332 is movable with regard to the pressure exerting belt 65 with the template 337 within the extent of the cutouts 338, wherein length of the cutouts is designed for the required scope of turning of the monitored person 2.

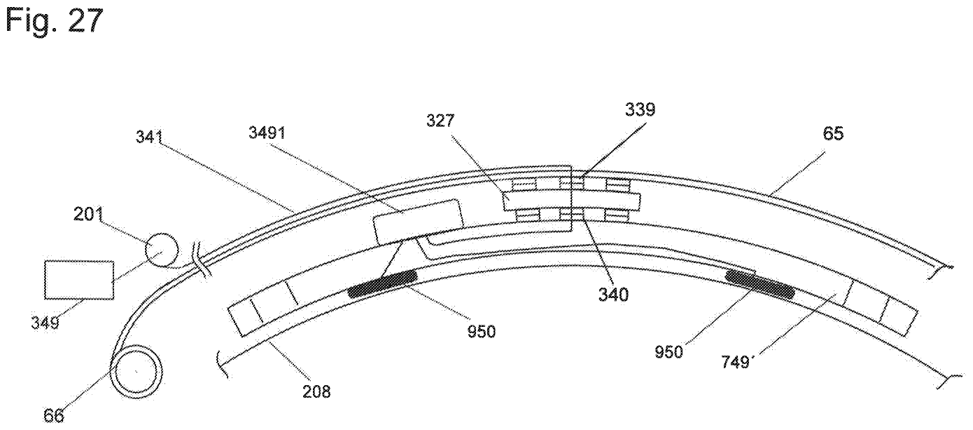

FIG. 27 shows connection of the chest belt 749′ and the pressure exerting belt 65 by means of a bridge 327 for routing of a “wired link 341 of the monitor” through the pressure exerting belt 65, “through the bridge between the connector 339 of the pressure exerting belt and the bridge”, through the bridge 327, “through the connector 340 of the chest belt with the bridge” and through the chest belt 749′ up to “the reduced monitor 3491 of cardiac signals”. In this embodiment, the monitor 3491 is powered by means of the “wired link 341 of the monitor”, wherefore it preferably does not comprise any accumulator and/or circuits for wireless communication. The cardiac signals scanned by electrodes 950 are processed in the monitor 3491 and transmitted further by means of the “wired link 341 of the monitor”.

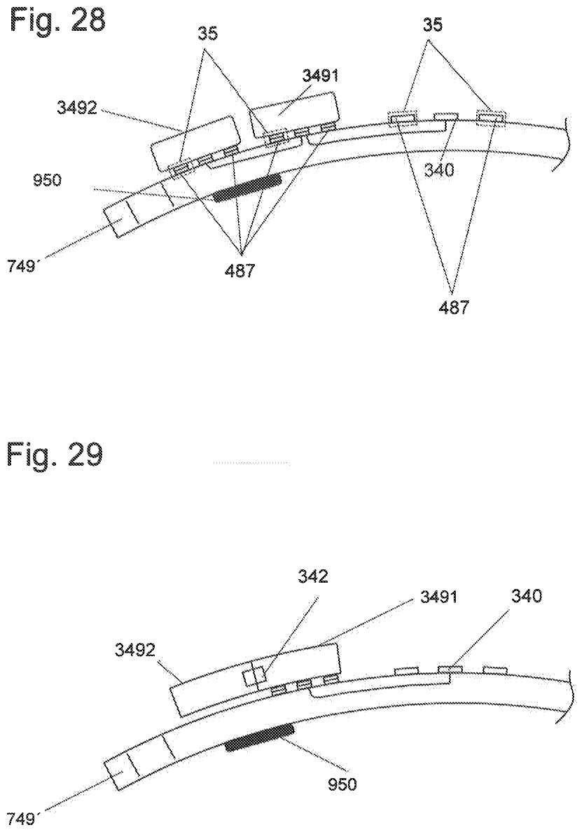

FIG. 28 shows fastening of the reduced monitor 3491 and the “supplement 3492 of the monitor” to the chest belt. The mechanical connection is solved by means of fastening elements 35, preferably snap fasteners 487. This solution allows the power supply of the monitor 3491 both via the “wired link 341 of the monitor”, as it was described in FIG. 27, and via the power supply from the “supplement 3492 of the monitor”. “The supplement 3492 of the monitor” preferably comprises an accumulator and communication circuits for wireless transmission.

FIG. 29 shows connection of the “supplement 3492 of the monitor” to the reduced monitor 3491′ directly, by means of the “connector 342 of the supplement”, which provides mechanical connection and electrical communication of both parts.

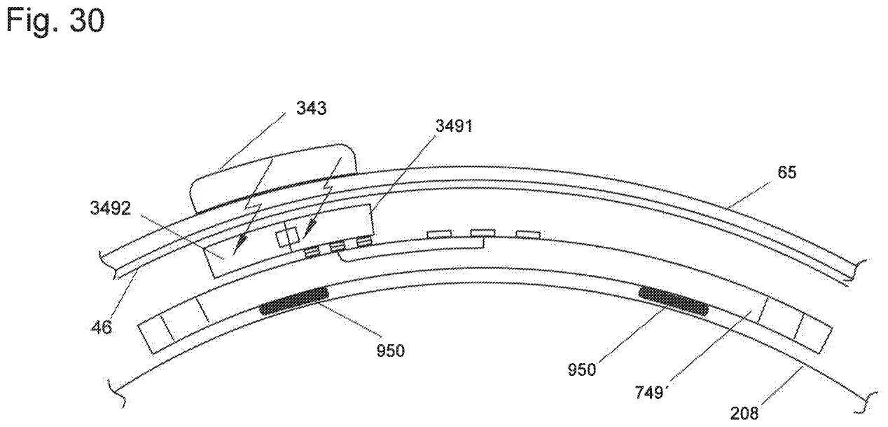

FIG. 30 shows the wireless recharging of the accumulator of the monitor in the working position by means of an induction charger 343. Such mechanical layout required close vicinity of the charger and the charged equipment. Preferably, the induction charger 343 is integrated in the pressure exerting belt 65 or it is fastenable to it. Power for its operation is supplied via conductors routed through the belt 65 from the power distribution system of the means 32.

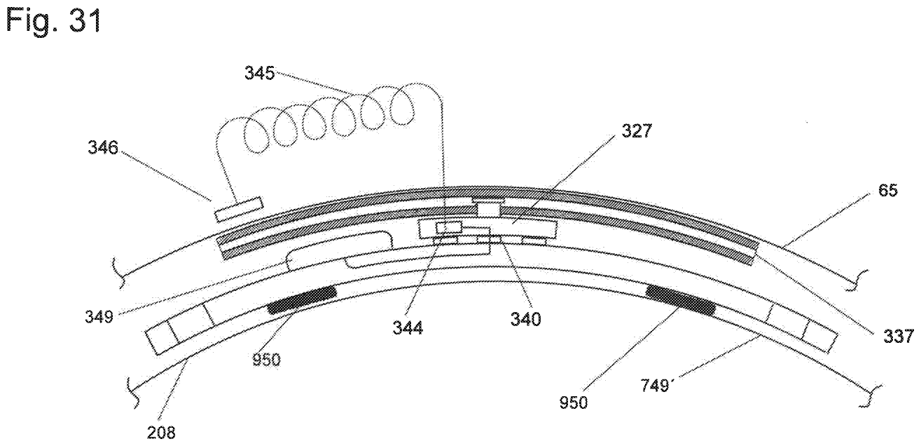

FIG. 31 shows interconnection of the monitor by an external cable. This arrangement is used in case that the pressure exerting belt is not provided with a connector connecting with the bridge 327. Cable 345 of the bridge is routed from the “connector 344 of the external bridge” to the “cable end 346” on the pressure exerting belt 65. From there through the belt 65 and/or on the surface to the power supply system of the means and/or to the controlling and displaying module 358, in case that the wireless transmission between the monitor 349 and the controlling and displaying module 358 is not used.

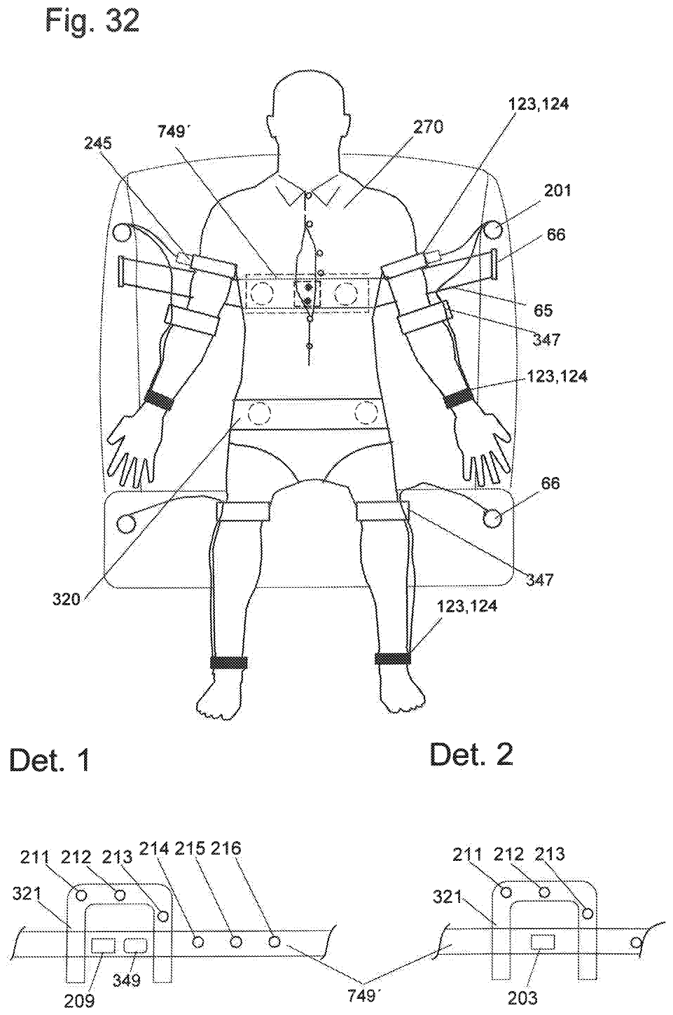

FIG. 32 shows a multi-lead measuring of the ECG of the monitored persons sitting in the driver's seat of a transportation or other means. In contrary to the previous figures here is an provided an enlargement consisting in scanning of signals of the left hand, of the right hand, of the left leg and of the right leg by means of electrodes, preferably connected with clips 124 and/or wrist bands 123 that press the scanning electrodes towards the wrist and ankles or other part of the limbs. These clips are preferably integrated to the seat, from which the monitored person 2 drives the means 32 controlled by the monitored person and from which they extract for fastening into the working position. After releasing from the working position on the limb the input cable is preferably wound to the clip 124 and/or the strap 123 preferably by a self-winding mechanism of the winch 66 into the seat body and the clip in to the holder of the clip. Preferably, the cables are routed over the auxiliary clips 347, which reduce the pull of the winches and hold cables along the limbs. Signals from 4 electrodes for scanning of limbs are transmitted to the monitor 349 according to its position either on the belt or in the driver's seat and/or in another place, preferably in the dashboard of the means 32. If the monitor 349 is placed on the shortened chest belt 749′, the signals are brought into the monitor 349 by conductors integrated into the pressure exerting belt that are routed into the bridge and further into the monitor. Alternatively, the signals are brought by conductors into the connectors on the shortened chest belt and/or directly to the monitor. To obtain the multi-lead, up to 12-lead, ECG it is possible to use the shortened chest belt disclosed in the Application PCT/CZ17/000012, which discloses electrodes for scanning of the leads V1 to V6. The shortened chest belt 749′ is attachable to the pressure exerting belt 65 by means of a bridge 332 detachably or, preferably, rigidly, what allows in case of not using the belt, once the driver disengages pressure exerting belt 65 that the pressure exerting belt 65 with the chest belt 749′ rolls up by means of winches 66 into its rest position on the seat side.

The above described configuration allows scanning of the one-lead ECG, either from the chest belt 349′ by using of the collecting electrodes according to FIG. 8 and/or from the signals coming from the upper limbs. If a multi-lead ECG is required, to the signals/leads LA, RA are added the ECG signals of the lower limbs LL, RL and from the chest belt V4, V5, V6 according to FIG. 9. The twelve-lead ECG is obtained by using of the chest belt with electrodes placed in a curve according to FIG. 1 Detail 2 or by using of the chest belt according to FIG. 10 or FIG. 18.

The input cables from the belts 123 and/or the clips 124 are preferably routed into the winches 201 of cables, so that only the required cable length is always unwound. Preferably the clips 124 and the straps 123 are provided with a connector 245 for disconnecting.

FIG. 32 Detail 1 shows alternative use of the chest belt 749′ with attached segment 321 of electrodes usable for twelve-lead ECG. The monitor 349 is placed on the belt and/or in the seat or in another place of the means. When it is placed on the belt 749′ signals of electrodes are brought to it by conductors routed in the belt and the segment 321 of electrodes, which is connected with the belt. Preferably, the monitor is powered by an additional accumulator 202.

FIG. 32 Detail 2 shows how signals from the electrodes are brought to the monitor 349 placed off the belt by means of a connector of the pressure exerting belt and by conductors routed through the pressure exerting belt.

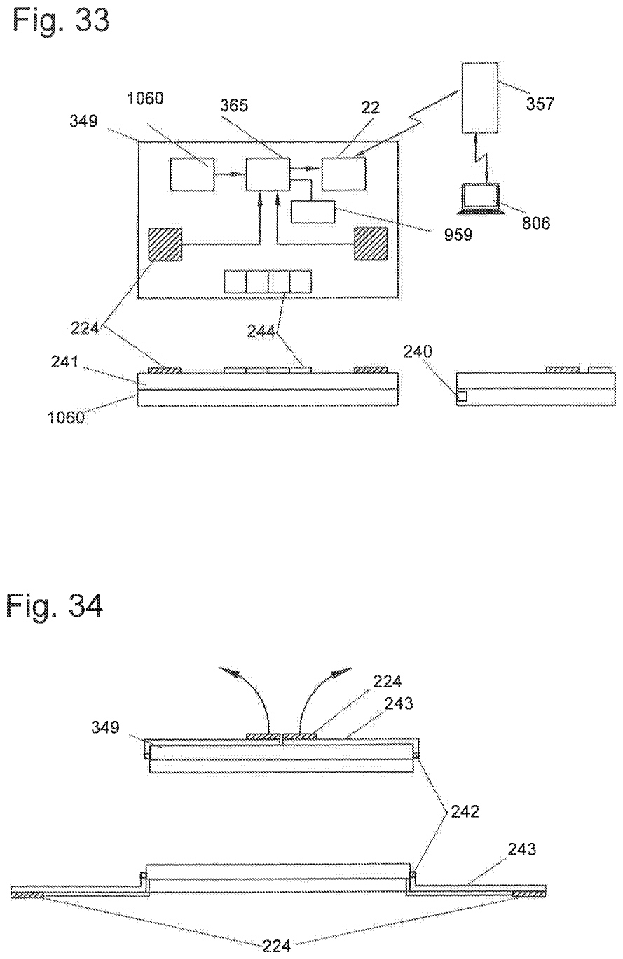

FIG. 33 shows the monitor 349 in thin design, implemented into the plate having the approximate size of a credit card, which monitor is portable very easily and which is suitable for instant measuring of the one-lead ECG in nearly any situation. On the monitor there are two contact areas 224 playing the role of electrodes. Further the monitor comprises a control unit 365 of the monitor, a communication module 22, control push-buttons 244, a SD card 959 and an accumulator 1060 of the monitor. The communication module 22 communicates with the operative controlling and displaying module 357, preferably a mobile phone and further with a server 806.

The contact surfaces 224 are attached to the chest and/or fingers touch the contact areas 224, one area is touched by a finger of the left hand and the other with a finger of the other hand. The integrated accumulator 1060 of the monitor is charged preferably over the charging contacts 240 and/or inductively.

FIG. 34 shows a monitor 349 implemented into the plate and provided with tipping plates 243 with contact surfaces 224, whereby after tilting longer distance from each other is reached by the electrodes formed by the contact surfaces 224, what is more preferable for scanning of ECG from the chest.

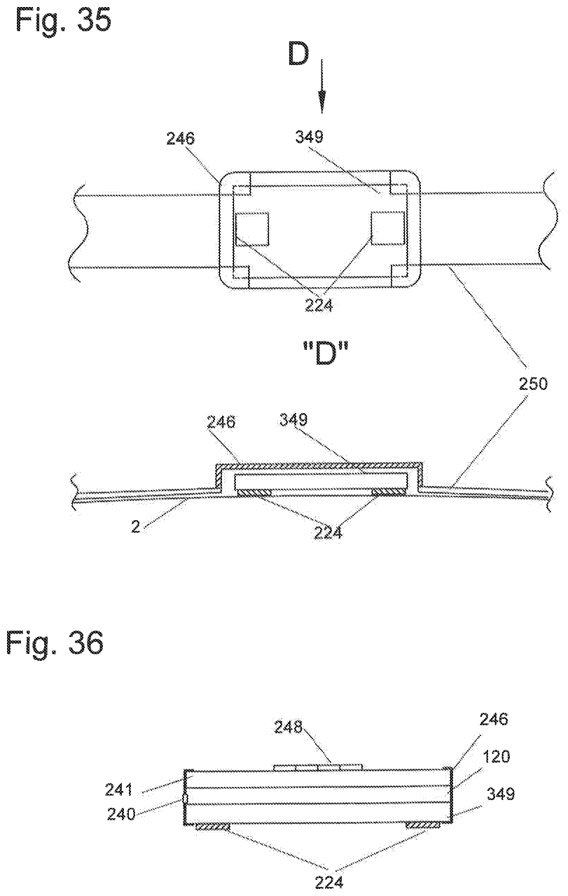

FIG. 35 shows a case 246 for the fastening strap 247 for fastening of the monitor 349 of the plate design and its fastening to the chest of the monitored person 2. The monitor 349 is inserted into the case 246, in which it is fixed and after fastening it to the chest of the monitored person 2 it can be used for long-term measuring of the ECG.

FIG. 36 shows an alternative of the monitor 349 in the plate design for the middle time measuring, when the case 246 is provided with additional accumulator 120, which via charging contacts 240 powers and charges. Alternatively, the additional accumulator 120 is replaceable and rechargeable over the cable or inductively. Preferably, the case is provided with a board with electronics and with control push-buttons 248 of the case.

FIG. 37 shows the power supply of the monitor 349 in the plate design in a case on the fastening strap 250 by means of modular additional accumulators 247. It is possible to place several of them on the fastening strap 250 according to the required necessary period of time for scanning of the ECG. At the same time, the additional modular accumulators 247 allow electrical connection for optimal power supply of the monitor. To the strap 250 they are fastened mechanically, preferably by snapping them or by a clip and/or they are connected with it by a connector and a strap 250 is provided with conductors for connecting of all modular additional accumulators 247 with the monitor 349. Alternatively, longer monitoring can be carried out by adding of an additional accumulator 120 of higher capacity.

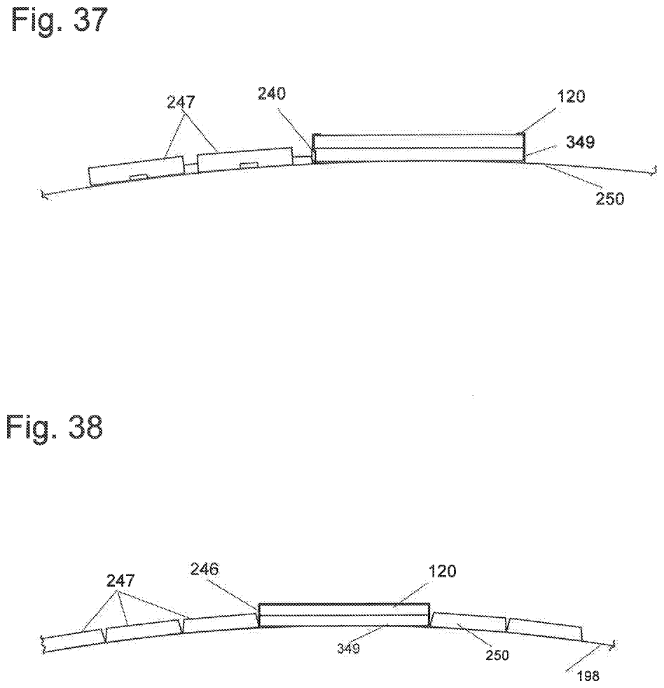

FIG. 38 shows a fastening strap 250 fastened to the case 246, formed entirely or partially by a line of modular additional accumulators 247 fastened together with stretchy modules to provide flexibility.

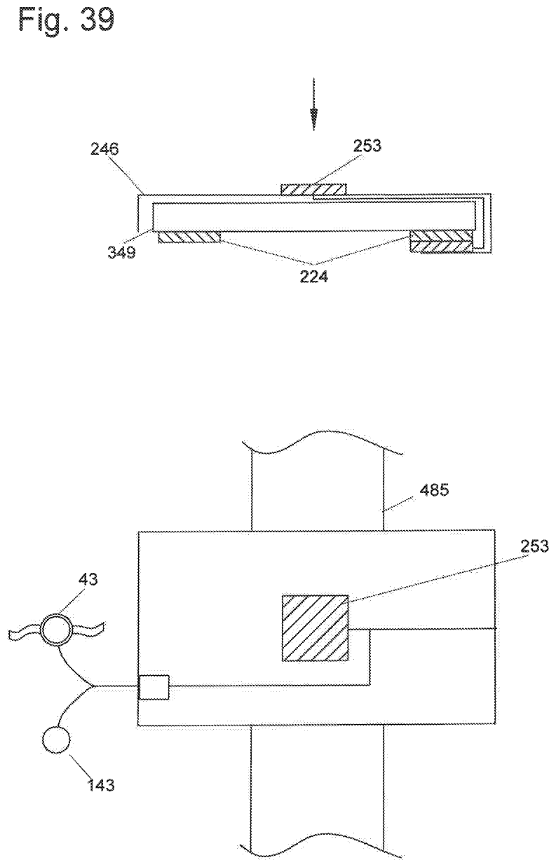

FIG. 39 shows a case 246 of the monitor for placing on the wristband. The case covers one contact area 224 and at the same time it is connected with it conductively and with a conductor this area 224 is connected with the contact area on the case side 246 turned away from the hand. The contact area not covered by the case 224 is in contact in the wrist area with the first hand of the monitored person 2. Once, the monitored person 2 touches the contact areas 224 brought to the top of the case 246, it is possible to monitor ECG for a short time. For the middle-time measuring, the monitor 349 is provided with a connector, and directly or via the case 246 it is interconnected with the electrode 143 glued on the other hand or with the wristband 43 put on the other hand.

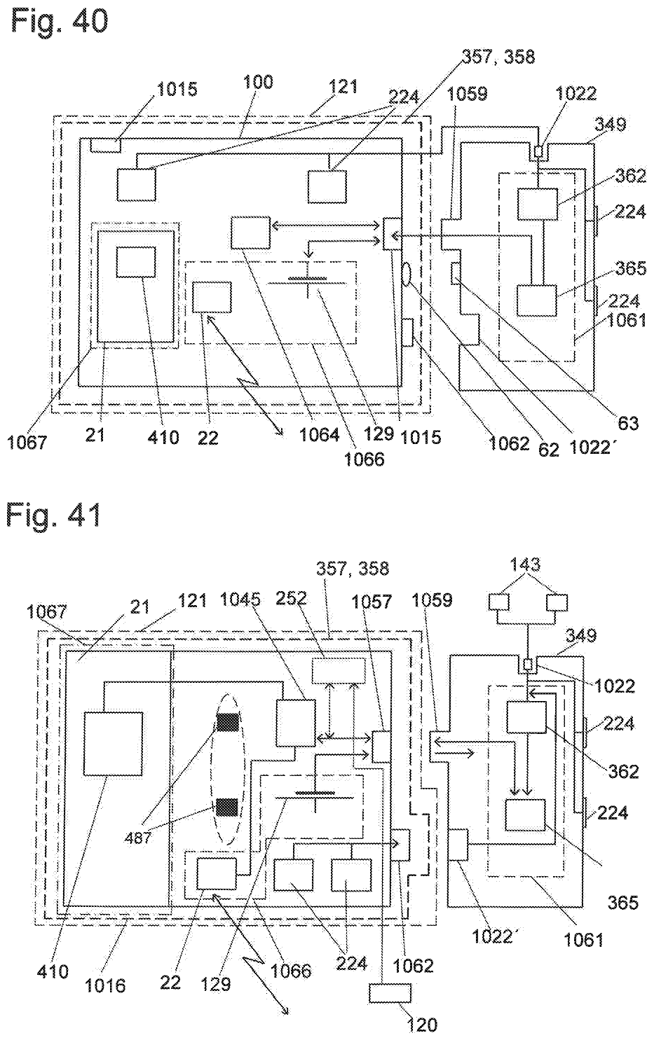

FIG. 40 shows a block diagram of the monitor and of the control module that can be linked by means of a connector into one functional whole. The basic model of the monitor 349 in a reduced form 3491 comprises only the block 1061 of the basic units, and this preferably comprising the front end 362 and the control unit 365. The block 1066 of supplementary units, which preferably provides the power supply by an accumulator 129 and communication by a communication block 22, is placed in the cooperating unit 121, where all optional functions are provided by the block 21 of additional equipment comprising additional units and parts and the module 410 of additional units. The cooperating unit 121, to which the monitor is 349 connected, is preferably formed in particular by the controlling and displaying module 358, the operative module 357, the controlling and displaying module or the base 1016 of the monitor. That the monitor 349 comprises only the block 1061 has advantages in small dimensions, low price of the monitor and in the possibility to select differently equipped cooperating units 121. The monitor 349 is connected for data transfer and power supply to the cooperating unit 121 by means of a connector 1059, preferably a USB, for power supply and data transfer. The contact surfaces 224 or the electrodes are preferably connected by means of the connector 1022 of electrodes. Alternatively, the electrodes are connected by means of springs 62, a strip, and the contacts 63 of the monitor or by connectors 1062 a 1022′, or through the cross-connecting connector 1015 and 1059 in case there is not any USB. Preferably, a second connector 1015′ is used, preferably a USB, which serves for data transfer and power supply for the module 357, 358, preferably formed by a mobile phone 100, as a common USB connector, which is preferably placed off the contact area of the attached monitor, to allow its use also when the monitor 349 is connected. The block 21 of additional equipment is both in the module 358, and in the operative module 357, so as in the base 1016 of the monitor replaceable for another, differently designed additional equipment. Replacement is carried out by means of the fastening mechanism 1067 of the block. The self-sufficient model of the monitor 349, which is relocatable, is to be used for independent operation, including the power supply. Off the basic units 1061, it comprises additional equipment 21, preferably including the module 410 of additional units. By means of the mounting elements 486, preferably formed by snap fasteners 487, the self-sufficient model is fastenable by mans of auxiliary equipment 64, preferably formed by the chest belt 749, 749′, or by the wristband 485 or by another pieces of auxiliary equipment, which preferably allow not only fastening, but also scanning of cardiac signals by means of contact surfaces 224 placed on them.

By insertion of the basic model of the monitor 349 into the base 1016 of the monitor, which is provided with mounting elements 486, preferably formed by snap fasteners 487 that allow to put the basic monitor 349 on the auxiliary equipment 64 the basic model of the monitor 349 fulfills functions as a self-sufficient model.

The base 1016 of the monitor comprises electronics 252, and power supply and recharging of the base, which base controls the power supply from the additional accumulator 120 or from an induction charger 343 (not shown).

The self-sufficient model is produced preferably with various outfits of the additional equipment 21 or the block of additional equipment 21 with various outfits is replaceable, preferably by means of a connector, and preferable is that owing to the fastening elements 486, preferably formed by snap fasteners 487 this self-sufficient model of the monitor 349 is replaceable and placed on pieces of auxiliary equipment 64 as actually required.

FIG. 41 shows block diagram of connection of the reduced monitor 3491 with the base comprising supplement of the monitor 3492. The externally placed additional accumulator 120 allows long-term measuring of the ECG.

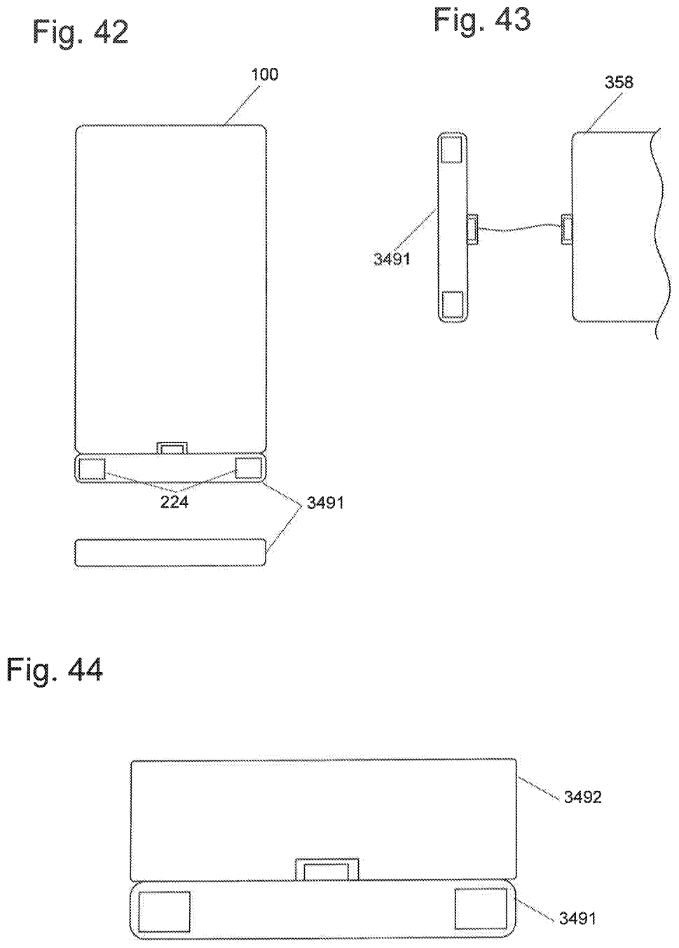

FIG. 42 shows connection of the reduced monitor 3491 with contact surfaces 224 to the mobile phone 100 by means of a connector, wherein the monitor is by form adapted to this connection.

FIG. 43 shows connection of the reduced monitor 3491 provided with the contact surfaces 224 with the controlling module 358 by means of a cable.

FIG. 44 shows a monitor 349 produced by connecting of the reduced monitor 3491 and of the supplement 3492 of the monitor suitable for short-time measuring of ECG.

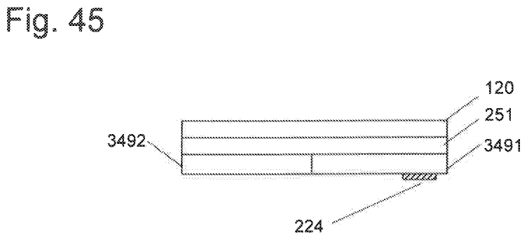

FIG. 45 shows a base of a monitor 1016 formed by a supplement of the monitor 3492 and of an additional accumulator 120 and by another board of electronics.

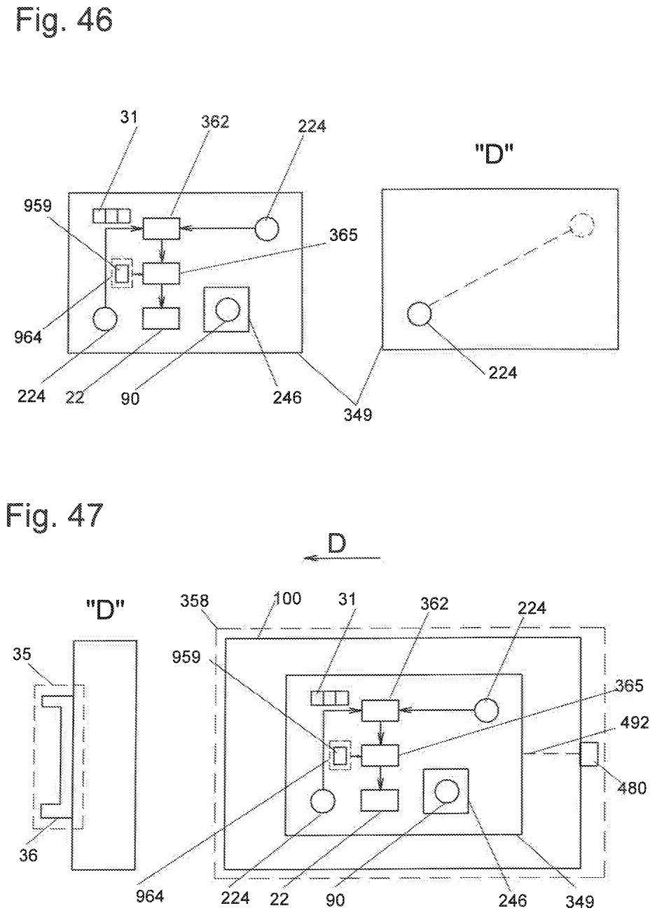

FIG. 46 shows the monitor 349 in the form of a card comprising contact areas 224, preferably placed opposite existing corners, preferably monitoring cardiac signals by touching by fingers or by the chest, where as an advantage appears to be their greater distance by placing in corners. They are interconnected to the front end 362 for analogue processing of cardiac signals and for conversion to the digital form for further processing in the digital control unit 365 of the monitor 349, wherefrom they are transmitted to the input of the communication module 22 for wireless transmission, to the controlling and displaying module 358 or to the operative controlling and displaying module 357 or to the server 806, as it is shown in FIG. 15, and/or they are saved on an extractable memory medium 964, preferably on a SD card 959. The so equipped monitor 349 is adapted for independent scanning of cardiac signals, their processing a transmitting further to the cooperating unit or saving in a memory.

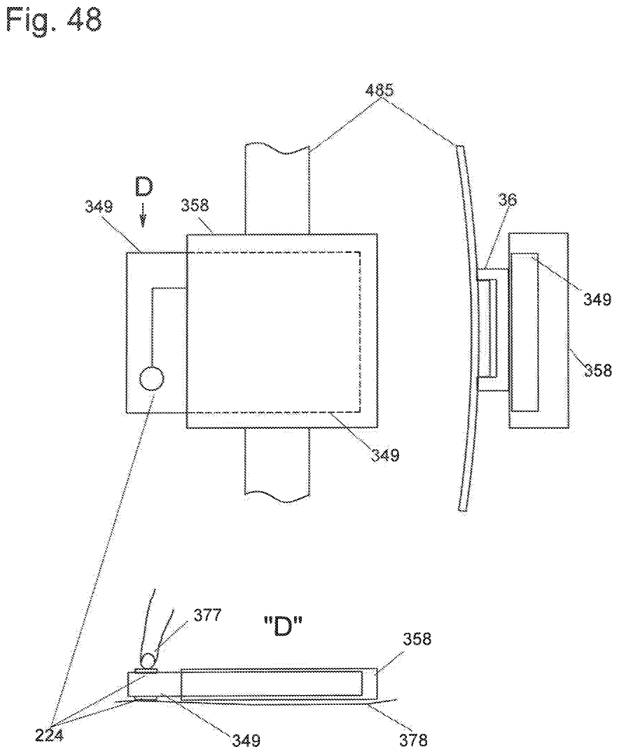

The view in the “D” direction shows the opposite side of the monitor 349, preferably with the opposite contact area 224 placed above the contact area 224 on the reverse side of the monitor 349 for scanning from the wrist interconnected to a distant contact area 224 in the opposite corner of the monitor, where a finger of the other hand is touched to the electrodes of the contact area 224 on the reverse side and preferably pushes the opposite electrode of the contact area 224 to the wrist, as it shows FIG. 48. The monitor 349 comprises an accumulator 129, preferably placed extractably in a case 1026 for the accumulator. The monitor 349 is controlled by the cooperating unit, preferably by the module 357, 358 and/or a server 806. In case that it is controlled by means of controlling elements 31, preferably formed by push-buttons, it is adapted for operation as an independent unit, in which the processed cardiac signals are stored on a memory medium.

FIG. 47 shows a monitor 349 or a sensor of a desktop monitor in the form of a card, which is fastened by a fastening element 35 to the controlling and displaying module 358, preferably formed by a mobile phone 100. The fastening element 35 preferably a withdrawable one, preferably formed by a sticky means, e. g. by a double-sided adhesive tape or by a Velcro hook-and-loop fastener or by a holder 36 for fastening of the monitor, preferably withdrawably fastened, preferably glued to the module 358 shown in a view in the direction “D”. The monitor 349 sends further cardiac signals by means of a communication module 22 into the module 358, preferably formed by mobile phone 100. Alternatively, the monitor is 349 interconnected by wired link 492 through the connector 480 of the module 358, preferably formed by the USB connector into the module 358. In this case, preferably the monitor does not comprise the following parts, and they are: an accumulator 90 of the monitor, a communication module 22, control elements 31, and a SD card 359, which are placed in the module 358 and used there.

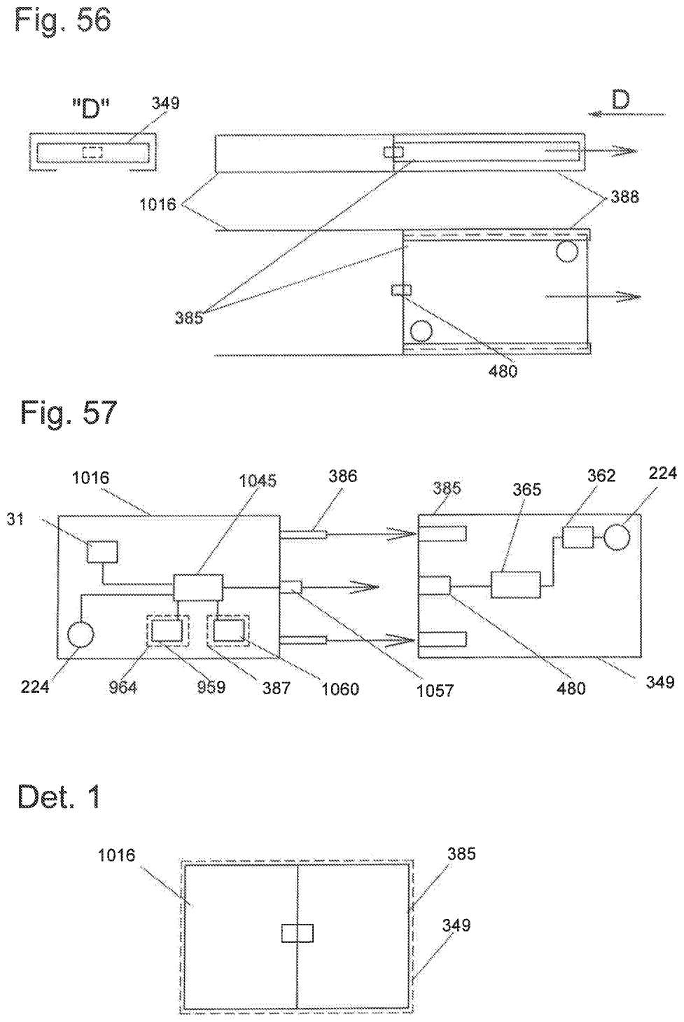

The monitor 349 without these parts becomes a sensor 385 of the monitor shown in FIGS. 56, 57, 58, and for independent operation it has to be supplemented by a base 1016 of the monitor shown in these figures.

FIG. 48 shows a monitor 349, fastened on the operative control and displaying module 357, preferably formed by a mobile phone 100 placed on a wristband 485, or by a smart watch 416. Fastening of the monitor 349 is preferably implemented withdrawably, preferably by a fastening element 35, preferably formed by an adhesive means, preferably a double-sided adhesive tape or by a Velcro hook-and-loop fastener, or preferably, the monitor 349 is adapted for inserting it into a holder, so as it is shown in view in the direction “D”, preferably placed withdrawably on the operative module 357.

Preferably the monitor 349 in the form of a board exceeds the operative module 357, and so it makes accessible the contact area 224 for touching it by a finger 377 of the other hand, which preferably pushes the contact area 224 on the reverse side of the board on the wrist of the first hand for scanning of cardiac signals, preferably already pushed also by action of the wristband 485.

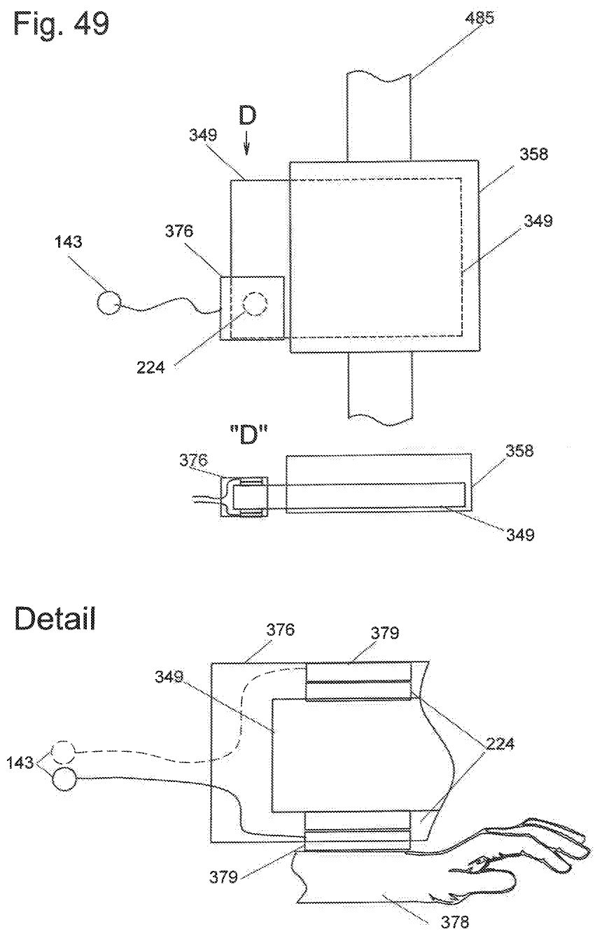

FIG. 49 shows putting on of the cross-connecting element 376 to the overlapping part of the monitor 349, to which cardiac signals are transmitted by a cable from the electrodes 143, preferably electrodes glued to the chest or from the electrode on the wristband placed on the other hand to the contact areas 224 of the monitor by means of the counter-contact 379, shown in detail 1, The glued electrode 143, preferably placed on the chest at a place recommended for scanning of signals on the other hand. The signals of the first hand are monitored by a contact area 224 on the reverse side of the monitor 349 attached to the wrist of the first hand, which is not covered by the cross-connecting element. Alternatively, the cross-connecting element 376 is connected also to the contact area 224 and the reverse one, and it transmits cardiac signals from two electrodes 143, preferably placed on the chest for scanning of signals of the first and the other hand to the contact area 224 and to the reverse contact area 224.

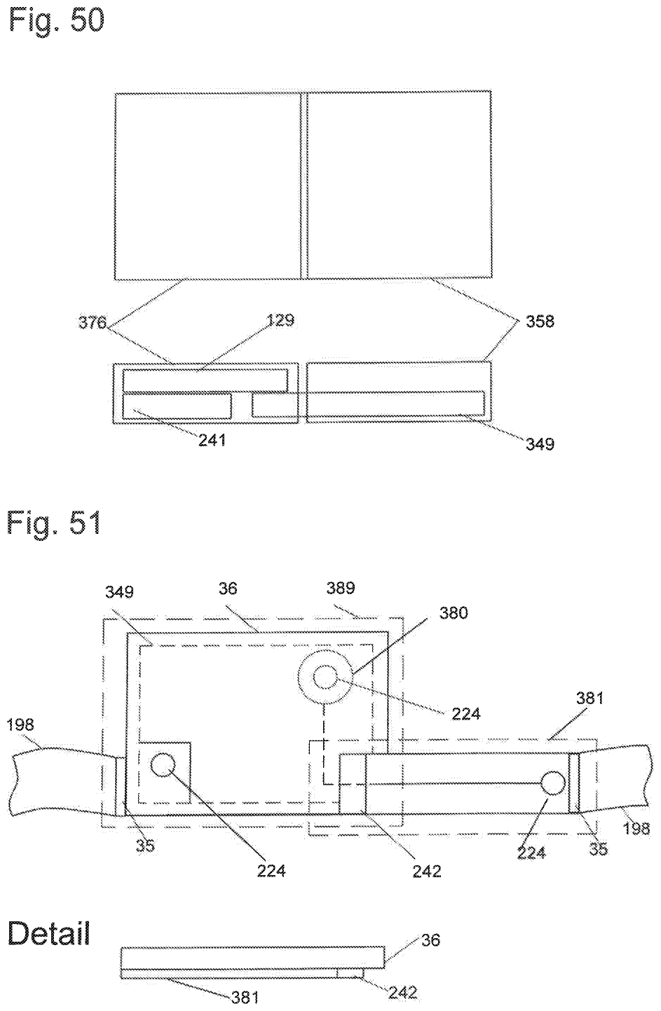

FIG. 50 shows a cross-connecting element 376 provided with additional accumulator 129 with a board 241 of the charging electronics increasing the capacity of accumulators 129 of the monitor, preferably replaceable. In this case the accumulator 129 of the monitor preferably forms a bridging accumulator providing power supply during the period of time when the additional accumulator 120 is replaced.

FIG. 51 shows a monitor 349 placed in a fastening means 35 preferably formed by a holder 36 into which it is preferably inserted. Fastening element 35 is adapted for fastening remote contact areas 224 remotely from of the monitor 349 and so the remote contact areas 224 for scanning of cardiac signals attaching to the chest and to connecting of the remote contact areas 224 to nearer contact area 224 on the monitor, which it covers. Preferably is implemented by a holder with a fastening element 35 preferably formed by a plastic extension, preferably withdrawably fastened hinge 242 to a holder 36, by means of which it is adapted for tilting over the holder, as it shows Detail 1. The contact area on the fastening element 35 is preferably interconnected with the contact area 224 on the monitor preferably by means of wired connection and the opposite contact to holder or connector during inserting of the monitor 224 do the holder or connector during inserting of the monitor 224 to the holder 36. The connecting members 375 are adapted for connecting of a stretchy strap 198 for permanent connecting of the monitor 349 to the chest for permanent monitoring.

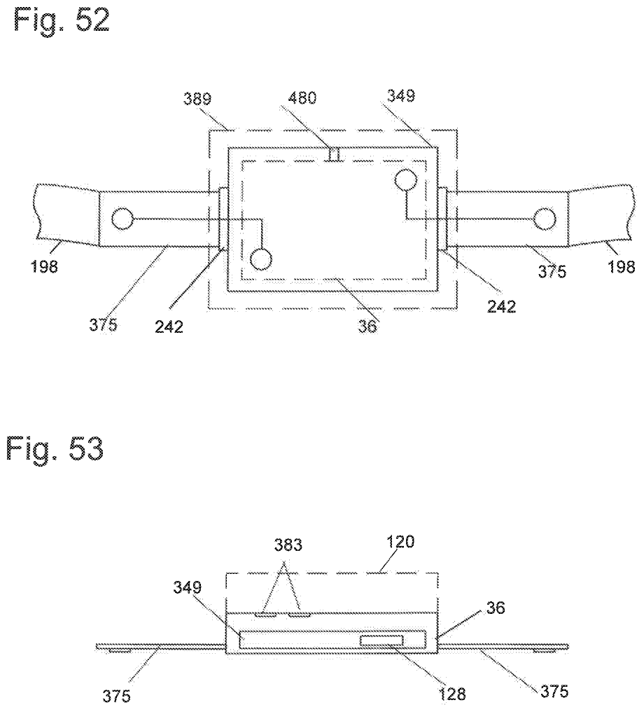

FIG. 52 shows an example of the fastening means 389 preferably formed by a holder 36 with extension member 381, where the inserted monitor 349 is connected by a connector 480.

FIG. 53 shows additional accumulator 120 connected for increasing of the accumulator capacity, which is connected with a monitor 349 by means of cross-connecting surfaces 383 to the holder 36 an the monitor that are in contact with each other when the monitor 349 is inserted.

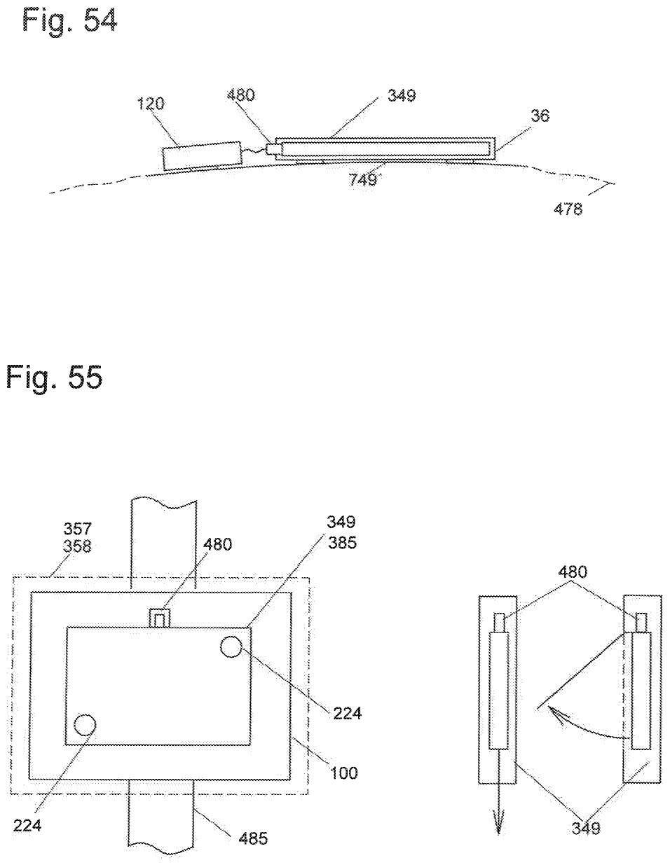

FIG. 54 shows additional accumulator 120 preferably withdrawably fastened on the chest belt 749 with contact areas 224, connected to the monitor 349, which is connected by the connector 480 to the additional accumulator 120.

FIG. 55 shows monitor 349 or sensor 358 of the monitor, preferably extractably placed in the operative module 357, 358, which is connected to it by connector 480, where extracting to Detail 1 is preferably carried by sliding out, to Detail 2 preferably by opening the door and extracting.

FIG. 56 shows communication of the sensor of the monitor 385 with the base by means of inserting into the holder 388 of the sensor, which is an extension of the base, where by interconnecting by the connector 480 is formed a monitor 349.

FIG. 57 shows connection of a sensor of the monitor 385 to the base 1016 of the monitor, preferably by inserting on spikes 386 and connector. Detail 1 shows the inserted monitor 349.

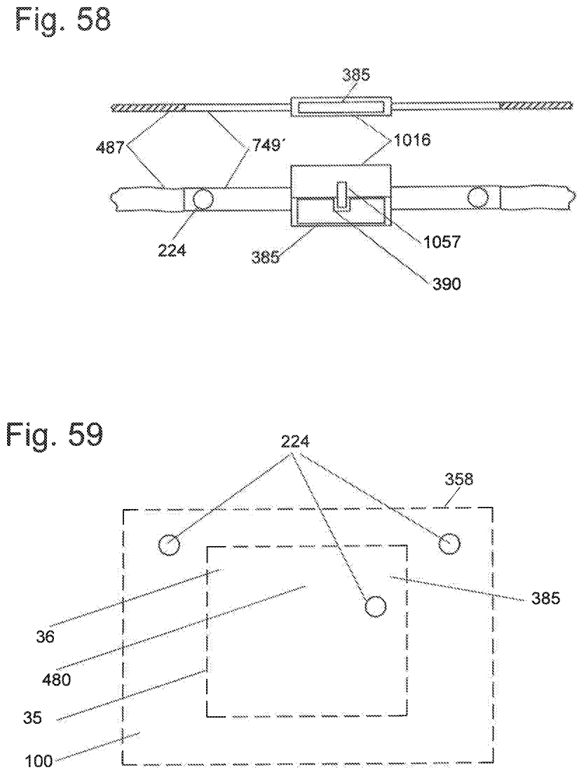

FIG. 58 shows a sensor of the monitor 385, which is fastened to the base 1016 of the monitor, which base is connected to a shortened chest belt 749′ with contact surfaces 224 for attaching to the chest.

Sensor 385 of the monitor is interconnected with the base 1016 of the monitor by means of the connector 390 of the sensor of the monitor and by the connector 1057 of the base. Withdrawably is attached an elastic strap 478, which strap allows permanent monitoring by fastening it to the chest.

FIG. 59 shows a sensor 385 of the monitor 349, which is fastened by a fastening element 35 to the module 358, which module is preferably formed by a mobile phone 100 together with a holder 36, with a connector 480, into which the sensor is inserted. From the connector a cable is routed to the module 358 serving for communication. The contact surfaces 224 are placed on the sensor 385 and/or on the module 358.

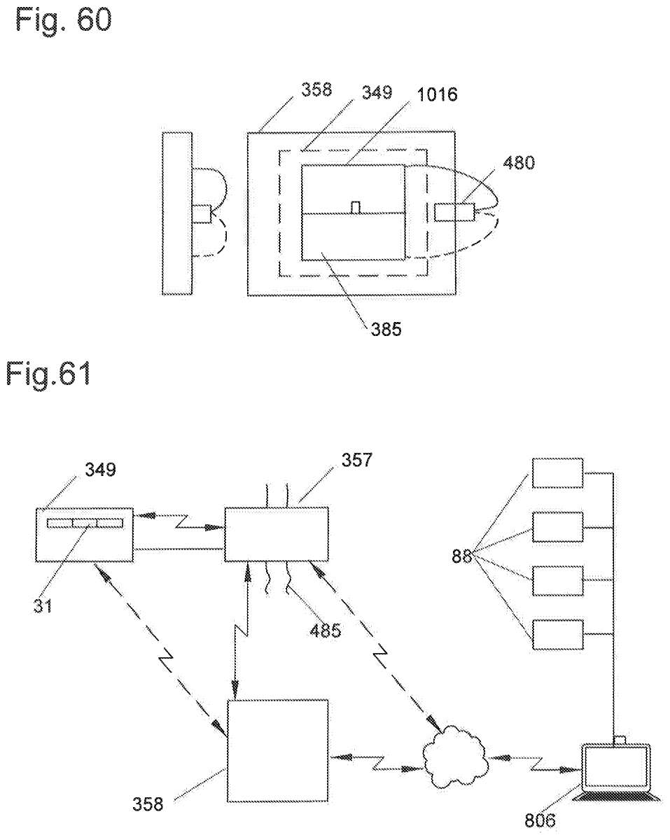

FIG. 60 shows a monitor 349 consisting of the joined sensor 385 of the monitor with the base of the monitor 1016 of the monitor, which is connected by a cable to the connector 480 to the module 358 or the monitor communicates by means of a communication module wirelessly.

FIG. 61 shows the monitor 349, communicating by wires or wirelessly with the operative module 357 or 358 for displaying and/or control. Preferably, they communicate with each other and exchange data about the processed cardiac signals and/or from a phone call. The modules 357, 358 transmit data about the processed cardiac signals further to a server with access rights for the remote users.

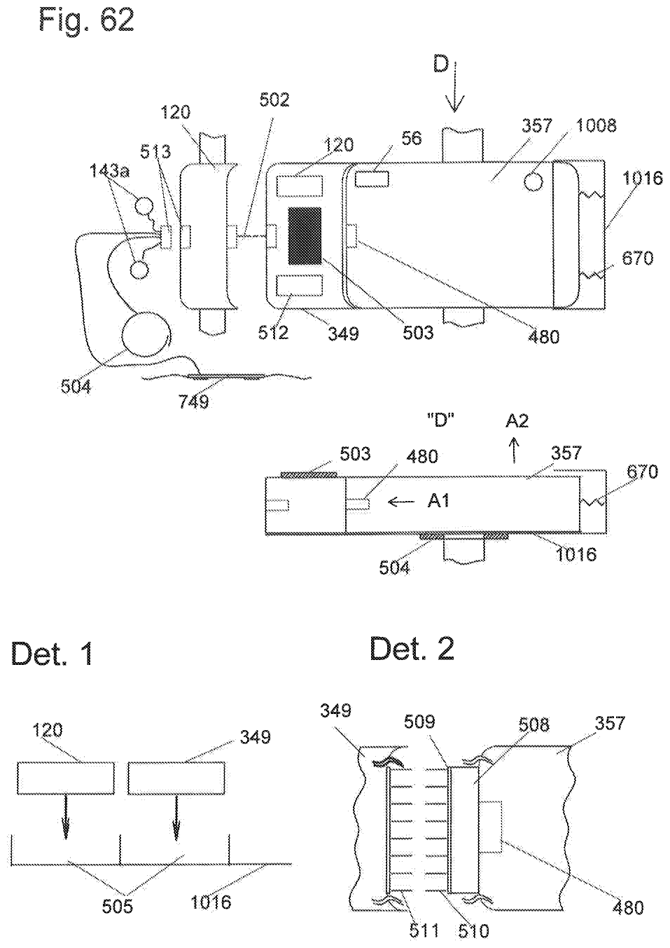

FIG. 62 shows the monitor 349 connected with the operative controlling and displaying module 357, preferably formed by mobile phone or by a smart watch, wirelessly or by wires by means of a connector 480, preferably USB, for data transfer on health data, and this at least about one of the following list: ECG, values of pulse and/or heart rate curves with limits of the regular heart rate, arrhythmia, variability. The monitor 349 is fastened on the base 1016 of the monitor, preferably withdrawably, a it is interconnected with the electrode 504 of the first hand, preferably in the bottom part of the base, which sits on a wrist. The electrode 503 on the other arm is placed preferably on the top part of the monitor for touching by a finger or a wrist, for what it has sufficient surface area. In case of interconnection of the monitor 349 by means of the connector 480 with the controlling operative module 357, the monitor 349 preferably is not provided with an accumulator and a wireless communication unit, wherein it is powered by the accumulator of the operative module 357 and communicates by means of the connector 480. Preferably,

An additional accumulator 120 is connected to the monitor 349 by means of a connector, optionally over the cross-connecting cable 502 of the additional accumulator 120, wherein the additional accumulator 120 is preferably fastened to the lengthy base 1016 and/or to a strap on the wrist. A cable is preferably connected to the connector 513 on the accumulator for connecting it to the electrode 503 on the other arm placed on the wristband on the other arm or glued to chest or to the electrode 504 of the first hand and the electrode 501 of the other hand on glued electrodes on the chest or on the chest belt.

In case that the accumulator 120 is not used, the connector of the cable is inserted into the connector 480 of the monitor.

The view in the direction “D” shows an example of extracting of the operative module 357 from the base 1016 by shifting it in the direction of the arrow A1 and subsequently by tilting it in the direction of the arrow A2. The spring 670 holds the operative module 357 pressed to the connector 480 v the inserted position. In the extracted position the operative module is adapted for making phone calls via a mobile operator's network.

Detail 1 shows base 1016, on which accumulator 120 and/or monitor 349 are placed withdrawably, in this example by insertion them into shelves 505 of the base.

Detail 2 shows a reducer 508 inserted into the connector 480 provided on the contact surface 509 with the monitor 349 with the first contacts 510 contacting with the second contacts 511 provided on the monitor 349 in the plugged state of the operative module 357, what allows easier insertion than when plugged by the connector 480.

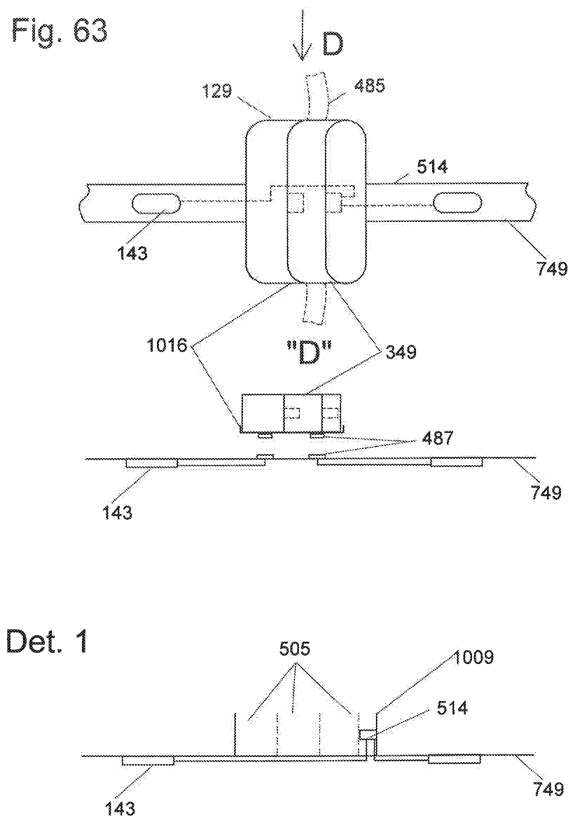

FIG. 63 shows repositioning of the monitor 349 preferably together with the accumulator 129 to the chest belt 749 by means of the base 1016, into which they are inserted. The base 1016 is placed on the belt preferably by means of snap fasteners 487. The monitor is electrically and mechanically interconnected withdrawably with the base 1016 and is connected to the electrodes 143 on the chest belt.

Detail 1 shows as another example the base 1016 fastened on the chest belt 749 with shelves 505 for inserting of the accumulator 129 and of the monitor 349 interconnected directly to the electrodes 143 of the chest belt.

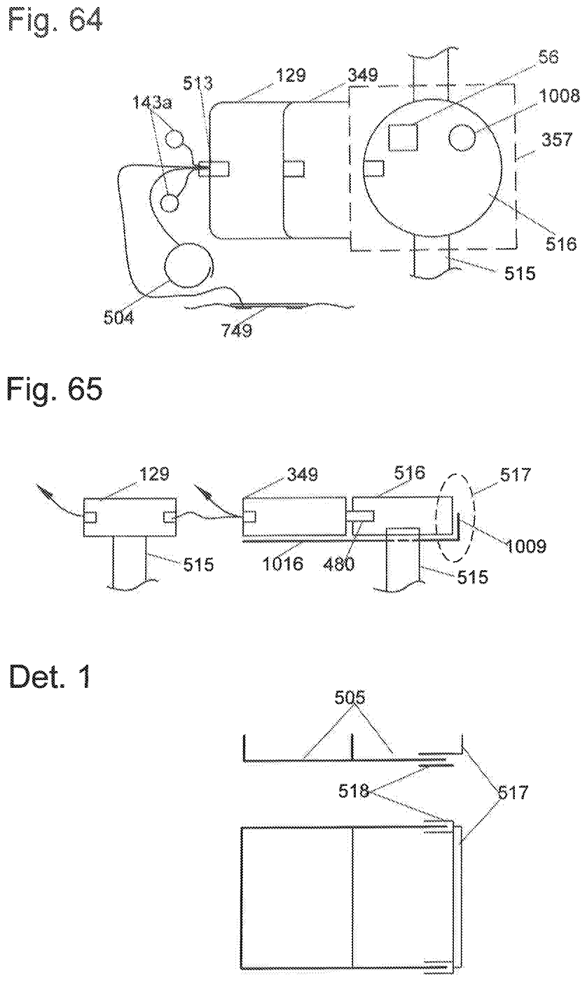

FIG. 64 shows monitor 349 preferably inserted as extractable into the monitor 349 formed preferably by extractable smart watch 516, preferably also with inserted accumulator 129. The principles are the same as in case of the monitor 349 formed by mobile phone.

FIG. 65 shows smart watch 516, which pushes the base 1016 to a wrist by action of the strap 515 tightened to the wrist, which base comprises fastening adjusting 517, preferably formed by a protrusion 1009, which prevents taking off of the base from the space under the smart watch 516, whereby the monitor is with the base fastened. Communication is wireless or by means of the connector 480. The monitor 349 is connected permanently in the base 1016, or withdrawably by means of a shelf 505 situate off the shelves for the smart watch (detail 1).

Detail 1 shows adjusting 517, which moves out by means of a telescopic mechanism 518 for adjusting of the base length for various modules for versatility.

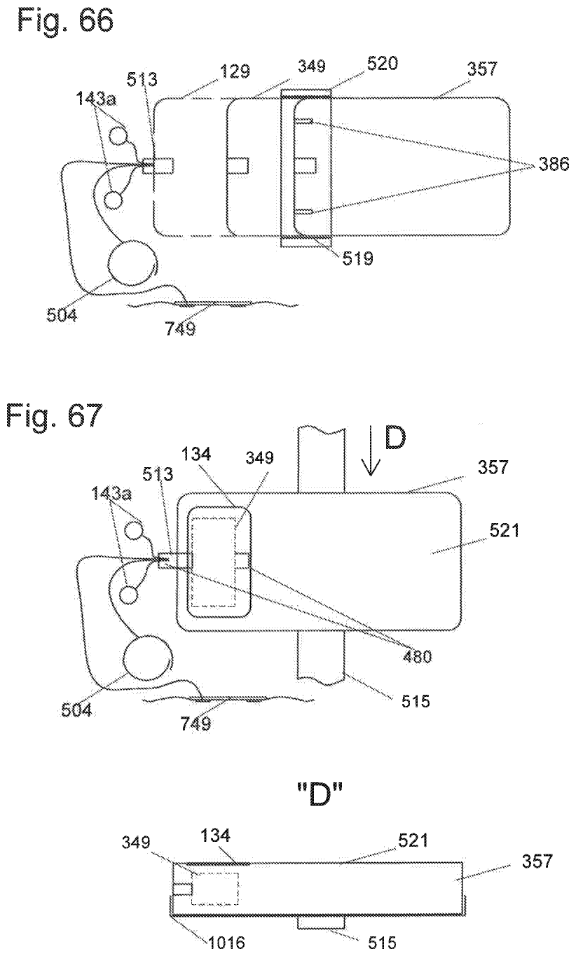

FIG. 66 shows monitor 349 fastened to the operative module 357 by means of plug-in, fastening mechanism 520 preferably formed by embracing jacket 519 of the module or inserted spikes 386. Optionally, an accumulator 129 is put on the monitor.

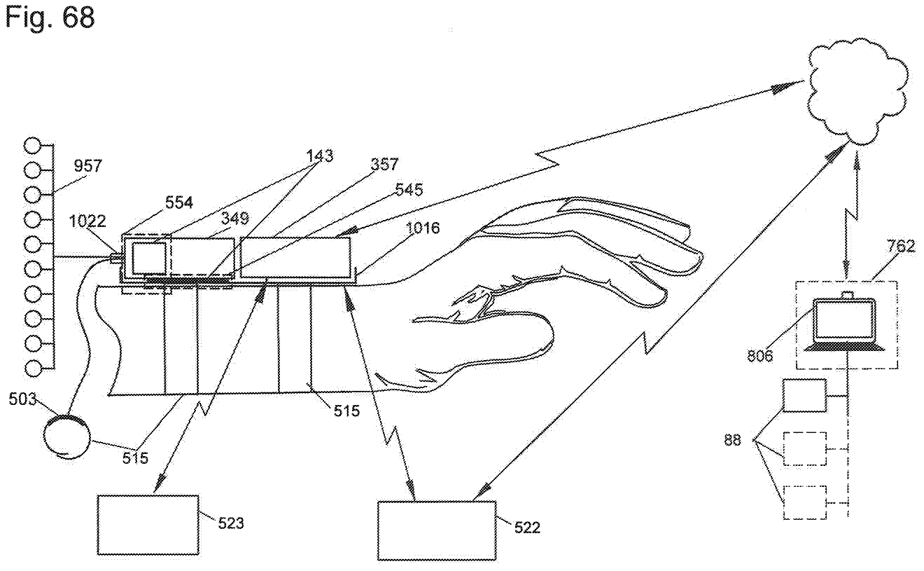

FIG. 67 shows a monitor 349 placed in the operative module 357 permanently or as extractable after tilting away of the door 134 or cover 521 a shifting out the connector 480 or it is dismountable. The extractable monitors 349 are portable to the chest belt 749. To the monitors electrodes can be attached for the first and/or the second hand and an additional accumulator by means of the connector 480. Preferably, the module is fastened to the strap 515 placed on a wrist preferably withdrawably, preferably by means of the base 1016.

FIG. 68 shows the control module 500 of smaller dimensions, fastened on the wrist by means of the base 1016 with a connected monitor 349, accumulator 129 electrode 503 of the other hand on a strap 515, where the monitor 349 and/or the control module 500 and/or displaying module 522 communicates over the mobile network, preferably formed by a mobile operator's network and/or over the WiFi network with server 806, preferably with server of the surveillance center 762 and with connected participants 88 for transmission of health data from the monitor 349 of cardiac signals and/or from the block 523 of the monitor of the health data preferably formed of at least one piece of data from the following list: blood pressure gauge, blood oxygen sensor, body weight sensor, thermometer, pedometer, breath monitor, physical activity monitor, which reports an alarm when the person under surveillance is not active, fall monitor, condition monitor.

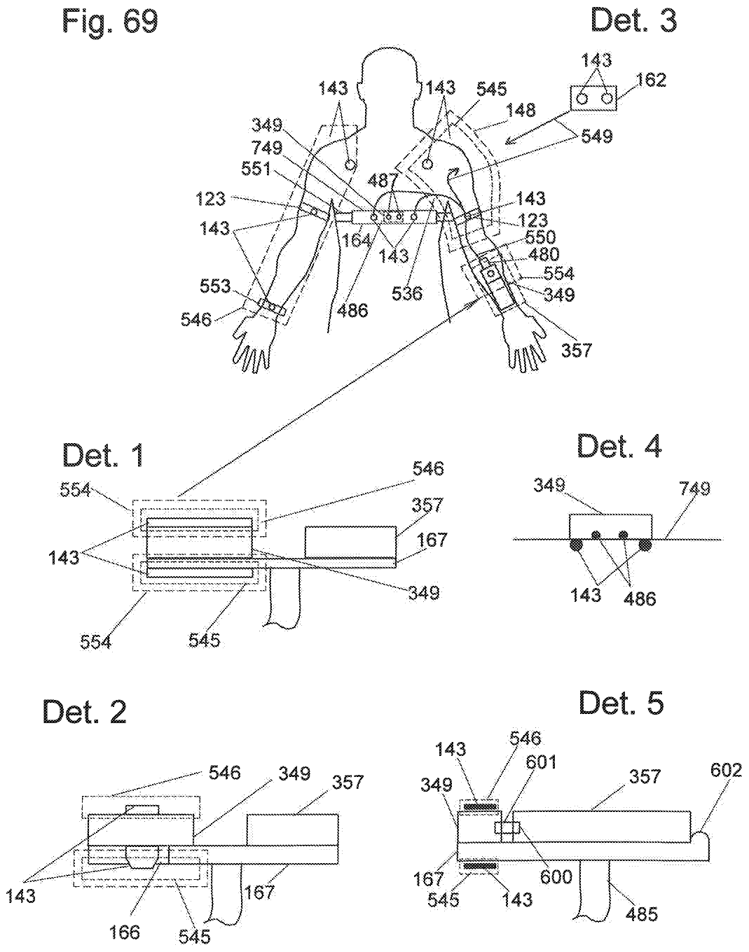

FIG. 69 shows electrodes for scanning of the one-lead ECG by means of the monitor 349 placed on a wrist.

For a short-time scanning, preferably the electrodes 554 of the monitor are used, wherein one of the electrodes 143 is placed on the bottom side of the fastening board 555, on which the monitor is placed 349, see Detail 1, or on the bottom side of the monitor 349, see Detail 2, for contact with the wrist of the left hand belonging to the system 545 of electrodes of the left hand and the second electrode 143 is placed on the top part of the monitor 349 and serves for contact with fingers or the wrist of the to the right hand belonging system 546 of electrodes of the right hand.

For a contact with the wrist system 545 of electrodes of the first hand preferably formed by electrodes 143 placed on the bottom side of the monitor 349, the fastening board 555 is provided with an opening 166 at the place of electrodes 143, which opening allows contact of the electrode 143 with the wrist, as it is shown in the detail 2.

To avoid the necessity, in particular during a long-term monitoring, to hold the right hand on the electrode 143 on the system 554 of internal electrodes of the monitor placed on the monitor 349 on the left hand, preferably the signals of the right hand are scanned by the system 546 of electrodes on the other hand instead of attaching the fingers to the monitor 349, preferably placed on the right hand on the wrist, preferably fastened by tapes 553 on the wrist or by clips, or they are fastened preferably by tapes on arms 123 or they are fastened by gluing on the right shoulder or on the right side of the chest.

For signals of the left hand, instead of the system 554 of internal electrodes of the monitor, alternatively, a system 148 of external electrodes glued on the left shoulder or on the left side of the chest or fastened by a belt on an arm is used.

For a short-time scanning from the chest, which gives less disturbed signal than scanning from arms the monitor is 349 adapted for scanning from a plate 164 with electrodes 143, which is attached to the chest, connected to the monitor 349 by a cable 549 by means of a connector 480 or the monitor is 349 adapted for repositioning from the wrist to a plate 164 with electrodes by means of a connector and for connecting to the electrodes 143. For the long-term scanning from the chest, with a monitor 349 is connected a chest belt 749 with electrodes 143, either by a cable 549 or from the wrist, the monitor 349 is moved to the chest belt 749, which monitor 349 is fastened to the belt 749 by mounting elements 486, as it is shown in Detail 4, and preferably it is connected wirelessly with the operative module 357, or a chest belt with own monitor 349 is used.

For the long-term scanning from arms during movement the curve of ECG is preferably dampened in the software to filter off the disturbing signals, to suppress disturbing produced by muscle activity, with the result of smoothening not only of the disturbing, but also of the details in the ECG curve, such as for example of the “P” wave or of the tiny fibrilation waves. If a detailed curve of the ECG is required it is scanned at rest and the ECG curve is filtered less or not at all. In this way the degree of filtration, preferably of the ECG curve, is adapted by various degree of damping according to various degrees of movement, for example for rest, walking, running, and the maximal damping for the extreme movement, wherein the degrees of damping are preferably switched over by a controlling element, which is placed on the monitor or off the monitor, preferably on the operative module 357, as required. For the less disturbed signal, also during movement, allowing lower degree of filtration, and thus a more detailed ECG curve, it is preferable to use the monitor 349 for scanning from the chest by the chest belt 749. As the scanning by the chest belt is not so comfortable as the scanning from the wrist, for common monitoring, at first, the more comfortable scanning from arms, wrist or fingers is used, preferably with a higher degree of filtration by means of software to allow scanning of ECG also during movement or from fingers. When ECG deviations from the standard have been found, whereby, a requirement to have high-quality ECG curves arises, ECG scanning, at best from the chest, at rest and at lower degree of damping by software is carried out.

Therefore, for the orientation monitoring is used preferably the more damped ECG, what allows monitoring in movement and for comfort monitoring by means of signals from hands, and for the detailed monitoring, for example when deviations from the standard ECG were found, the less damped or undamped ECG signal is used, preferably scanned from the chest, and the degree of damping is variable. Detail 5 shows a fastening board with a monitor 349, preferably withdrawable, which board is placed under the module 357 on the wristband 485. On the monitor electrodes 143 of the system 545 of electrodes of the first hand and the system of electrodes 546 on the other arm are placed. Or, the monitor 349 is fastened permanently to the operative module 357 by a fastening element 600 and it is connected by the connecting element 601, which is preferably connected for data transmitting to the operation module 357, or it is built into the operation module 357. Preferably, the monitor 349 is connected with the operative module 357 wirelessly.

Preferably, the operation module 357 is formed by one device from the following group: a smart watch, a mobile phone on a wristband. Preferably, the operative module 357 is withdrawable from the wristband, in particular for calling. Preferably, it is built in.

Preferably, the monitor is wirelessly interconnected with the controlling and displaying module 358, preferably formed by a mobile phone, and the operative module 357 may display, receive SMS messages or calls from the module 358.

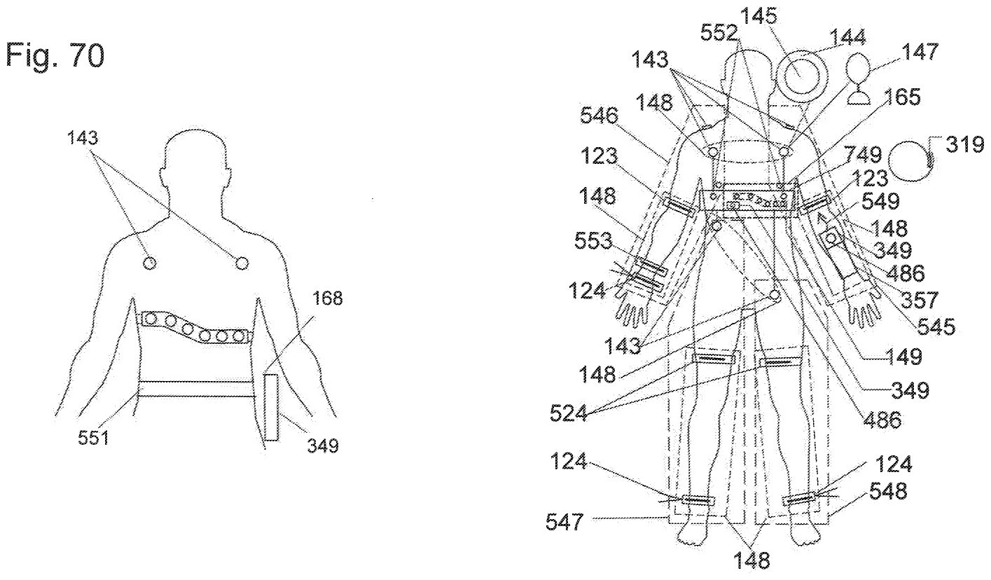

FIG. 70 shows scanning of the one-lead ECG by the monitor 349 for preliminary diagnosis and the multi-lead ECG is used for a more detailed diagnosis if necessary, preferably it is carried by a universal monitor 349 adapted both for the one-lead and the multi-lead scanning, for example when deviations from the normal are found during the preliminary diagnoses.

Preferably, for the sake of simplicity, the one-lead scanning is arranged as shown in FIG. 69. Preferably, the universal monitor 349 is used, which monitor is adapted for scanning of the multi-lead ECG or another monitor 349, additionally to the monitor 349 for the one-lead scanning, is used for scanning of the multi-lead ECG.

For easy replacement or repositioning of the monitor 349, preferably mounting elements 486 are used.

For the multi-lead scanning by two electrodes 143 from the system 545 of electrodes of the left hand or the electrodes 143 of the monitor for the left hand and the system 546 of electrodes of the right hand, preferably used already for the one-lead scanning,

the system 548 of electrodes of left leg and a system 547 of electrodes of the right leg that are fastened by tapes 524 on legs or clips 124 or by gluing by above legs connected cables 159 to monitor 349 are added. Preferably they are placed on the wrist for measuring of up to six-lead ECG, wherein to the values measured for the leads I and II the values the lead III and the leads aVR, aVL aVF are calculated.

For scanning of the 7-lead to 12-lead ECG, preferably the plate 164 with electrodes 143 for V1 to V6, preferably connected by a cable-549 to the monitor 349, preferably placed on the wrist, is added. In case of one electrode 143 on the plate 165, optionally it is possible to monitor some of the leads V1 to V6 according to place to which the plates 165 are attached or by subsequent moving of all leads V1 to V6.

In case of two electrodes 143 on the plate 165, it is possible to monitor optionally 2 leads from V1 to V6, and therefore, with three actions of repositioning to scan all leads V1 to V6. In case of 6 electrodes 143 on the plate, 164 it is possible to monitor the leads V1 to V6 at once. Preferably, the plate 164 is produced from an elastic material to accommodate to the chest shape and the plate is to be used for holding on the chest for the short-time scanning. For the long-term scanning the plate 164 5 preferably fastened to the embracing strap 551 for permanent fastening to the chest, whereby a chest belt 749 is formed.

Alternatively, the monitor is preferably placed directly on the plate 164, wherein it is preferably moved for the multi-lead scanning from the wrist and the cable 549 transfers signals from the electrodes 143 on hands and legs to it, or the system 148 of external electrodes and/or the system 149 of electrodes on the belt with optional number of electrodes 143 from the one-lead up to the twelve-lead ECG is used. Preferably, the electrodes 143 of the system of external electrodes 148 are moved to the chest belt 749 as required to reach the state that majority or all electrodes are placed on the chest belt 749, as it is shown in FIGS. 8 to 10 and this preferably with using of the bridge 79, the additional board 204, or the springs 150, or it may be placed or moved preferably to the belt of trouses, when supplemented by a buckle 168 for fastening.

In case, the monitor 349 is also for the one-lead scanning fastened to the chest belt for the long-term scanning, it is connected to the system 545 of electrodes of the first hand and the system 546 of electrodes of the other hand is connected by the cable 549 to the monitor. For the multi-lead scanning, optionally are added the system 547 of electrodes of the right leg and the system 548 of electrodes of the left leg for lead III and the leads aVF, aVL, aVR, and as required, optionally, electrodes for V1 to V6, preferably on the chest belt. In case, the one-lead monitor 349 is used, for the multi-lead ECG the monitor is replaced by a multi-lead one, preferably by fastening it by means of the mounting element 486.

The electrodes 143 fastened to chest are preferably glued by gel 145 on a target 144, or fastened by suckers 147. The electrodes on arms or wrists are preferably fastened by tapes 123 or clips 124 or on the monitor 349.

Preferably, at start of the scanning for the sake of simplicity, preferably the one-lead ECG is used for the preliminary diagnosis using higher damping of the ECG by increased filtering to allow monitoring also from hands and during movement, and when deviations are found from the standard the multi-lead ECG is scanned without damping by filtering, or the one-lead or the multi-lead ECG is scanned from the damping during movement and after termination of damping at rest to obtain greater details on the display.

Another advantage is for the preliminary diagnosis to monitor and evaluate the curve of the heartbeat, preferably with limit curves of the regular heartbeat, without damping or with only moderate damping of the signals by means of a software, and when deviations from standard are observed, to monitor the one-lead or the multi-lead ECG, as required during movement with higher damping and/or at rest without damping.

### INDUSTRIAL USE

The universal chest belt is usable for monitoring of persons, for monitoring of their health state at rest and/or at performance of various activities, and further for prevention of arrhythmia, and also for medical use.

1. A universal ECG monitor, characterized in that a monitor (349) is placed on a chest belt (749) and is adapted for scanning of cardiac signals from minimally one lead of ECG by means of a system (149) of electrodes on the chest belt (749), and further it is adapted for enhancements for scanning of a greater number of ECG leads, wherein for informative scanning all electrodes are placed on the chest belt (749) together with a display, which display approximately corresponds to the ECG display and said universal ECG monitor is adapted for monitoring and processing of cardiac signals to a ECG curve and for their monitoring and evaluating in at least one evaluation unit (138), preferably formed by an operative module (357) and/or a displaying module (358) and in case of finding of deviations from the standard ECG courses or if a more accurate ECG display is required, the universal ECG monitor is adapted for connecting of a system (148) of external electrodes for providing of a more accurate scanning a displaying of the ECG.

2. The universal ECG monitor according to claim 1, characterized in that a right collecting electrode (190) and a left collecting electrode (191) serve for scanning of the one-lead ECG.

3. The universal ECG monitor according to claim 1 characterized in that a RA electrode (194) and a LA electrode (195) are placed on a chest belt (479).

4. The universal ECG monitor according to claim 1 characterized in that a right collecting electrode (190) or a Kranz's clip comprised of resistors (218) connected to the RA electrode (194) and to the LA electrode (195), placed on a chest belt (479), are used instead of the Wilson's clip, whereby it is possible to place all electrodes necessary for scanning of the leads V3 to V6 on a chest belt (749).

5. The universal ECG monitor according to claim 1, characterized in that a RL electrode (196) is placed on the chest belt (749).

6. The universal ECG monitor according to claim 1, characterized in that all electrodes for scanning of an up to 12-lead ECG are placed on a chest belt, and a bridge (27) of electrodes except the electrode (197), which is placed off the chest belt (749).
